SEQUENCE LISTING (23 sequences)

SEQ ID NO: 1 - DNA, 8426 bp, Artificial Sequence
  Transgenic DNA sequence
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagagtcgaggtcgactctagacttaattaaggatcccccccaaccgtcgcatcctgtgcaactctggtcagtggccgttttgtgttgaatgttctccaccaagagcgcatggctgcggaagcgaggtgcagaccgaggtcccgagggatcgccctccgggaagcggtgatgctgttgttgtacttcggggtgccaaccgggcactcctacaacctggacccggagaatgcactgctgtaccagggcccctccggcacgctgtttggctactcggtggtgctgcacagccacgggtcgaagcgctggctcatcgtgggggctcccactgccagctggctctctaatgcctcagtggtcaatcctggggcgatttacagatgcgggatcagaaagaatccaaaccagacctgcgaacagctccagtcgggtagccccagtggagagccttgtgggaagacatgcctggaggagagggataaccagtggctgggggtcaccctttccagacagcctggagaaaatggctctatcgtgacttgtgggcacaggtggaaaaatattttttacatgaagagcgataacaaactccccactggcatttgctacgtcatgccttctgatttgcggacagaactgagtaaaaggatggccccgtgttacaaagattatacgagaaaatttggagaaaattttgcatcatgtcaagctggaatatctagtttttacacacaggatttaattgtgatgggggccccgggatcatcgtactggactggcaccgtctttgtctacaatataactacaaaccaatacaaagcatttgtagacagacagaaccaagtaaaatttggaagctacttaggctactcagttggagctggacattttcgaagtccacatactaccgaagtcgtgggaggagcccctcaacacgaacagataggaaaagcatatatatttagcattgatgaaaacgaactgaacatcgtatatgaaatgaaaggtaaaaagcttggctcatactttggagcttctgtctgcgctgtggacctcaatgcagatggcttctcagatctccttgttggagctcccatgcagagcaccatcagggaggaaggaagagtattcgtgtacatcaactctggcatgggagctgtgatggttgaaatggaaagggtccttgtcggaagtgacaaatatgctgcaagatttggggagtctatagcgaatcttggcgacattgacaatgacggctttgaagatattgctattggtgcaccacaagaagacgacttgagaggtgctgtctacatttacaatggccgagtcgatggaatctcctccacctactcacagagaattgaaggacagcaaatcagcaaatcattaaggatgtttggacaatctatctcaggacaaattgatgcagacaacaatggatatgttgatgtagccgttggtgcatttcaatctgattctgcagtgttgctaaggacaaggcctgtagtgattgttgaagcatctttaagccatcctgagtctgtaaataggacaaagtttgactgtactgaaaatggacttccatctgtgtgcatgcatcttacactgtgtttctcatataaaggcaaagaggtcccaggctacatcgttttgttttacaatgtgagcttggatgtgcacaggaaggcagagtctccgtcaagattttatttcttctctaatgggacttctgacgtgattacaggaagcatacgagtttcaagcagtggagagaaatgtaggacacaccaggcattcatgcggaaagacgtgcgagacatccttacccccattcatgtagaggccacataccaccttgggcatcatgtgatcaccaaacgaaacactgaggaatttccaccactccagccgatccttcagcagaagaaagaaaaagacgttattagaaaaatgataaactttgcaaggttttgtgcctatgaaaattgctctgctgatctccaagtttctgcaaaagttggatttttgaagccatatgaaaataaaacctatcttgctgttgggagcatgaagaccataatgctaaacgtgtccttgttcaacgctggcgatgatgcttacgaaaccactctgaatgtccaactccccacaggcctttatttcattaagatcttagacctggaagagaaacaaataaactgcgaagtgactgagagctcaggcatagtgaagcttgcctgcagcctaggttacatatatgtggatcgcctctcaaggatagacattagctttctcctggatgtgagctcactcagcagggcacatgaggacctcagcatcagtgtgcatgcctcctgtgaaaacgagggcgaattggaccaagtgagggacaacagagtgaccttaacgatacctctaaggtatgaggttatgctgactgttcatgggcttgtgaacccaacttcatttgtgtatggatctagcgaagaaaacgagccagaaacatgcatggccgagaagctgaacctcactttccatgttataaacactgggattagcatggctccaaatgttagtgtgaaaataatggtaccaaattcttttctccctcaagatgataagttgttcaacgttttggatgtccagacaactacagggcaatgccattttaaacactatggaagagagtgtacatttgcacagcaaaaaggcatagcggggacgttgaccgatatagtcaaattcctatcaaagactgataagagactcctgtattgcatgaaagctgatcaacactgtttagatttcttatgcaatttcggaaaaatggaaagtgggaaggaagccagcgttcatattcagctggagggcaggccatccatcttggaaatggatgagacctcatcactcaagtttgaaataaaagcaacagcttttccagagccacacccaaaagttattgaactaaataaagatgagaacgtggcccatgttttcttggaagggctccatcatcaaagacccaaacgacatttcaccatcattattattaccatcagcttgctacttggacttattgtacttttattaatttcatgtgttatgtggaaggctggattctttaaaagacagtacaaatctatcctacaagaagaaaacaggagagacagctggagttatgtcaacagcaaaagcaatgatgactgaagacttctacactgagagaactgaaaaactcaggttaggaaaaagaaatcctgttcagaagacccgtcagaattatcgaattcctgcagcccgggggatccactagttctagagcactgcgatgagtggcagggcggggcgtaatttttttaaggcagttattggtgcccttaaacgcctggtgctacgcctgaataagtgataataagcggatgaatggcagaaattcgccggaggcggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttaaattaggcaaaggaattgctctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggcccccc

SEQ ID NO: 2 - DNA, 20 bp, Artificial Sequence
  primer
tcttctctttggccaaccgt

SEQ ID NO: 3 - DNA, 20 bp, Artificial Sequence
  primer
gcaggtctggtttggattct

SEQ ID NO: 4 - DNA, 19 bp, Artificial Sequence
  primer
cgcctgccagcaccggaca

SEQ ID NO: 5 - DNA, 20 bp, Artificial Sequence
  primer
agaggcggaggcgctgtgac

SEQ ID NO: 6 - DNA, 24 bp, Artificial Sequence
  primer
gctgaccgcttcctcgtgctttac

SEQ ID NO: 7 - DNA, 18 bp, Artificial Sequence
  primer
cagatcgcctggagacgc

SEQ ID NO: 8 - DNA, 18 bp, Artificial Sequence
  primer
caacttatcatcttgagg

SEQ ID NO: 9 - DNA, 18 bp, Artificial Sequence
  primer
tggctccaaatgttagtg

SEQ ID NO: 10 - DNA, 30 bp, Artificial Sequence
  primer
ggagtggatcctaggaaaggggataacatt

SEQ ID NO: 11 - DNA, 63 bp, Artificial Sequence
  primer
ggccagtgaattgtaatacgactcactatagggaggcggtttttttttttttttttttttttt

SEQ ID NO: 12 - DNA, 20 bp, Artificial Sequence
  probe
gtcaagatgctaccgttcag

SEQ ID NO: 13 - DNA, 5085 bp, Mus musculus
gaccttggactgagcaaaggggcaacctggggacctggttgtattctttgcctttcccagtctccctcttctccctcattggaagatccactctgggccatgaggcttctcctgcttctggcttttatctctgtcatccctgtctctgttcagctattggacgcgaggcaatttttaatctataatgaagatcacaagcgctgcgtggacgctctaagtgccatctcagttcagacggcaacttgcaacccggaagctgaatcccagaaattccgctgggtgtcagattctcagatcatgagtgttgctttcaaattatgtttgggagtgccatcaaaaactgactgggcttccgtcaccctgtatgcctgtgattcgaaaagtgaatatcagaaatgggagtgtaagaatgacacactctttggaatcaagggcacagagttatattttaattatggcaacagacaagagaagaatatcaagctttacaaaggttcgggattgtggagcagatggaaggtctatggaaccacggatgacctgtgctcgagaggatatgaagccatgtactccttactgggcaatgcaaatggagccgtctgtgcatttccattcaagtttgaaaacaagtggtatgcagactgcacctctgccgggcgctcggacggatggctctggtgtggaaccaccactgactacgacaaagacaagctgtttggattttgtccattgcactttgagggaagcgagagattatggaacaaagatccactgactggcattctttaccagataaactccaagtctgctttaacctggcatcaggcaagggcaagctgcaagcagcagaatgctgacctcctgagtgtcacggagatccacgagcaaatgtacctcacaggattaaccagttccttgagctcgggactctggattggactcaacagtctgagtgtacgcagtggttggcagtgggctggaggaagcccattccggtatctgaactggctaccaggaagtccatcatcagagcctggaaagagctgtgtgtcactaaaccctggaaaaaatgccaagtgggaaaatctggaatgtgttcagaagcttggctacatttgtaaaaagggaaacaataccttgaacccatttatcattccctcagcaagcgatgtgcctaccggctgccctaatcagtggtggccctatgcaggccactgctacaggatccatagggaagagaagaagatccagaaatatgctttgcaagcttgtaggaaggagggtggggacctggcaagtatccacagcattgaggagtttgacttcatcttctcccagctcggatatgagccaaatgatgagctgtggattggtttaaatgacatcaagattcagatgtactttgagtggagtgatggaaccccagtgacatttactaaatggcttcctggagagccaagccatgagaacaacagacaggaggactgcgtggttatgaaaggcaaggatggatactgggcggacagagcctgtgagcaaccactaggttacatctgtaagatggtatcacaaagccatgctgtagtaccggagggtgcagacaaaggctgccggaaaggctggaaacggcatgggttttactgctacttgattggatccactctatccaccttcactgatgcaaaccacacatgcacaaatgaaaaggcttatttaacaacagttgaagacagatatgaacaagcattcctgactagtttggttggattgaggcctgaaaaatatttttggacaggactctcagatgttcaaaacaaagggacgtttcggtggactgtggacgagcaggtgcagtttacacactggaatgccgacatgccaggacgaaaggcgggatgtgttgccatgaaaaccggagtggcaggtggcttatgggatgttttgagttgtgaagaaaaggcaaaatttgtgtgcaaacattgggcagaaggagtgactcgcccaccagagcccacaacaactcctgaacccaaatgtccagaaaactggggtaccaccagtaaaaccagcatgtgtttcaaactgtatgcaaaaggaaagcatgaaaagaaaacgtggtttgaatctcgagatttttgcaaagctataggtggagagctggcgagcatcaagagtaaagatgaacagcaagtgatttggaggctgattacgagcagtggaagctaccatgagctgttttggttgggactgacctatggaagtccttcagaggggttcacctggagtgatggttctcccgtttcctatgaaaattgggcttacggtgaaccaaataattaccaaaatgttgaatattgtggtgagctgaaaggtgaccctggcatgtcctggaatgatatcaactgtgaacacctcaacaactggatttgtcagatacaaaaagggaaaacactactacctgagcccacacctgctccacaagacaatccaccagttactgcagatgggtgggttatttacaaagactaccagtactattttagcaaagagaaggaaaccatggacaacgcgcgacgattttgcaagaagaattttggtgatcttgctacaattaaaagtgaaagtgaaaagaagtttctatggaaatatataaacaagaatggtgggcagtcaccatattttattggcatgttaatcagcatggataagaaattcatttggatggatgggagcaaagtagattttgtggcttgggctacaggagaacccaactttgcaaatgatgatgaaaactgtgtaacgatgtacacaaattcagggttctggaatgacatcaactgtggttatccaaataacttcatctgccagagacataacagcagcatcaatgccactgccatgcctaccacacccacgacaccaggtggctgcaaggaaggttggcatttgtacaagaacaagtgctttaaaatttttggatttgctaatgaagaaaaaaaaagctggcaagacgcacggcaagcttgcaaaggactgaaaggaaacctggtgtccatagaaaatgcacaagagcaagcatttgttacctatcacatgagagactccactttcaatgcctggactgggctgaatgatatcaacgcagaacacatgttcctgtggacagctggacaaggagttcattatacaaactgggggaaaggctatcctggtggaagaagaagtagcctatcttatgaagatgctgactgtgtagttgtgattggtggcaattcacgagaggcagggacctggatggatgacacctgtgacagtaaacaaggctatatatgtcaaacacagactgacccttccctgcctgtttctccaaccactactccaaaagatggctttgttacatatgggaaaagcagctattcccttatgaaattgaagctaccatggcatgaagcagagacatattgcaaggatcatacttccctgcttgctagcattctagacccctacagtaatgcatttgcatggatgaaaatgcacccatttaatgtacccatatggattgccctgaacagcaacttgaccaataatgaatatacttggacggatagatggagggtgcggtacactaactggggtgctgacgagccgaagcttaagtcagcatgtgtttacatggatgttgatggctactggagaacatcatactgcaatgaaagtttttattttctctgcaaaaaatcagacgaaatccctgctactgaacctcctcaactgcctggcaagtgtccagagtcagaacagactgcgtggattcctttctatggccattgctactattttgaatcttcttttacgagaagttggggtcaggcttctctggaatgccttcgaatgggtgcctccctggtttccatcgagactgctgctgagtccagttttctgtcataccgtgttgaacctcttaaaagtaaaaccaatttttggataggcatgttccgaaatgttgaagggaagtggctttggttgaacgacaatcctgtctcctttgtcaactggaaaacaggcgatccctctggtgaacggaatgattgtgtagttctagcttcatcttcgggcctttggaataatatccactgttcttcgtacaaaggatttatttgtaaaatgccaaaaattattgatcctgtaactacacactcatccattacaaccaaagctgaccaaaggaagatggatcctcaacccaagggctcttctaaagcagcaggagtggtcaccgtggtcctcctgattgtgataggtgccggcgttgcagcctatttcttttataagaaaaggcatgcgttgcacatacctcaagaggccacctttgaaaacactctctacttcaacagtaatctgagtccaggaacaagtgacacgaaagatctcatgggcaacatcgagcagaatgagcatgcgatcatttagcatcctaatctgactgtctcatattcgaagttcaagaagtctgaactaaagttttcaattttttaattcaatatgattgtttctttttttaaaaaaaattagcactgggttgcattggtttgtcctttttcttcgcctaattgaacaataattgctcatttactatcctggcaagattagagaaacaaaaaacagaagagggataataatgttgattgttgattgccacttttgaagatacctttgatgaatacacagcactagcgtcttaacactgctacactggtggtaggtctcatgtcaaccctgcagatttcaagagttctagaaacaaccacgtaagcccactcagggtattttcaggacctcccagagatatgttatacaccgaattgtaattcaacatttaaaagtccatgttaaaatagacaagtccacataaccccagattgaataagaatgtattcaggtctttttcagcagtcctgattcatgtcagttattgagaactgtatttatccacatgtagaataataagctactgagaatcttgtttgtccccaggcaaggtttgacaggctgaatattgcaaatgtgttctgtgttctgttgtat

SEQ ID NO: 14 - protein, 13 aa, Artificial Sequence
  peptide
HSLGKWLGHPDKF

SEQ ID NO: 15 - DNA, 20 bp, Artificial Sequence
  primer
caattcacgagaggcaggga

SEQ ID NO: 16 - DNA, 22 bp, Artificial Sequence
  primer
gggaagggtcagtctgtgtttg

SEQ ID NO: 17 - DNA, 1055 bp, Mus musculus
atggtagcacgcaaccaggtggcagccgacaatgcgatctccccggcagcagagccccgacggcggtcagagccctcctcgtcctcgtcttcgtcctcgccagcggcccccgtgcgtccccggccctgcccgggggtcccagccccagcccctggcgacactcacttccgcaccttccgctcccactccgattaccggcgcatcacgcggaccagcgcgctcctggacgcctgcggcttctattggggacccctgagcgtgcacggggcgcacgagcggctgcgtgccgagcccgtgggcaccttcttggtgcgcgacagtcgccaacggaactgcttcttcgcgctcagcgtgaagatggcttcgggccccacgagcatccgcgtgcacttccaggccggccgcttccacttggacggcaaccgcgagaccttcgactgccttttcgagctgctggagcactacgtggcggcgccgcgccgcatgttgggggccccgctgcgccagcgccgcgtgcggccgctgcaggagctgtgtcgccagcgcatcgtggccgccgtgggtcgcgagaacctggcgcgcatccctcttaacccggtactccgtgactacctgagttccttccccttccagatctgaccggctgccgctgtgccgcagcattaagtgggggcgccttattatttcttattattaattattattatttttctggaaccacgtgggagccctccccgcctgggtcggagggagtggttgtggagggtgagatgcctcccacttctggctggagacctcatcccacctctcaggggtgggggtgctcccctcctggtgctcctccgggtcccccctggttgtagcagcttgtgtctggggccaggacctgaattccactcctacctctccatgtttacatattcccagtatctttgcacaaaccaggggtcggggagggtctctggcttcatttttctgctgtgcagaatatcctattttatatttttacagccagtttaggtaataaactttattatgaaagtttttttttaaaa

SEQ ID NO: 18 - DNA, 523 bp, Mus musculus
cattactctgctactatgctgacctgcactagtgcacccctgtgaggaaccccccatggcgagggcctctgcctctgacacacccttctcgctgccctgagccccggagcagccatgggaggcagcctgcatacctggaagccagctgcacagtgctgtggggaaacggaagcgttacagaccgggccaggctgcctggctgctgctccctctgggctcatagctggcccacttgacggtccctggtggcttctggagggaagctgtacttctggatacatttttcttttccttcagagaccgtgacagagggagctctgtcagcgggaagttcaggccaccaggagtagtgatttgtaagaaattactgtctcctcagtgaacctcctagtctctttatagacttaaccttcccttcagttaaagggtttctttttcggagggcctagactgtgctgaccttgtgtgaccagagatttaaagatgaaaaaaaacaataaatgtgtttacttctgcaaaaaaa

SEQ ID NO: 19 - DNA, 17 bp, Artificial Sequence
  primer
agcagccatgggaggca

SEQ ID NO: 20 - DNA, 18 bp, Artificial Sequence
  primer
tccgtttccccacagcac

SEQ ID NO: 21 - DNA, 496 bp, Mus musculus
ctcagtctgaatattcctggaaaaagatactgagtggagctgcagtgttcctgcttgggctgattgtcttcctggtgggggttgttatccatctcaaggctcagaaagcatctgtggagactcagcctggcaatgaggcctcaagagagagtctccattctcaaccctagtaagatcctaatggaagcctctcccccagagcctgaagaagtgtgacagggctgaggattcgggttggcaaaggatgctggctcttcagtctgtgagatgtggtcatggaccttccctttgtcctgtacctcagtagtccctcgtttccaggacatggtccagaaacttccttaggacctaactcattccacacccaagttgctttggcccaatcctaaccctgtgactgtggagccccaagatttctatcttccaaccacagcttctagtcttccatacagtcaccttaaaaatccttattgtaaaaataaagcagtcacaggca

SEQ ID NO: 22 - DNA, 19 bp, Artificial Sequence
  primer
gacagggctgaggattcgg

SEQ ID NO: 23 - DNA, 22 bp, Artificial Sequence
  primer
aggtccatgaccacatctcaca

(19) United States
(12) Patent Application Publication
(10) Pub. No.: US 2003/0154499 A1
(43) Pub. Date: Aug. 14, 2003

(54) Mouse unable to express functional alpha-4 integrin protein, and methods for assaying compounds or agents for alpha-4 integrin protein antagonist activity and a genetic marker for evaluating efficacy of modulators of signaling activity of a VLA-4 receptor

(76) Inventors: Monika Wasel-Nielen, Bridgewater, NJ (US); Bernhard Kirschbaum, Frankfurt am Main (DE); Martyn Foster, Loughborough (GB); Gregory Polites, Bridgewater, NJ (US); Olga Khorkova, Bridgewater, NJ (US); Bin Zhu, Bridgewater, NJ (US)

Correspondence Address: ROSS J. OEHLER, AVENTIS PHARMACEUTICALS INC., ROUTE 202-206, MAIL CODE: D-303A, BRIDGEWATER, PA, 08807, US

(21) Appl. No.: 10/163,899

(22) Filed: Jun. 5, 2002

Related U.S. Application Data
(60) Provisional application No. 60/297,112, filed on Jun. 8, 2001.
(60) Provisional application No. 60/382,927, filed on May 23, 2002.
(60) Provisional application No. 60/384,109, filed on May 29, 2002.

(30) Foreign Application Priority Data
Oct. 17, 2001 (GB) 0124895.4

(51) Int. Cl. G01N 33/00

(52) U.S. Cl. 800/3; 800/18; 435/455

(57) ABSTRACT

Provided herein is a mouse that is unable to express functional alpha-4 integrin protein, and methods for assaying agents for alpha-4 integrin antagonist activity, as well genetic markers for analyzing the efficacy of VLA-4 modulators, and particularly antagonists.

### DOMESTIC PRIORITY CLAIM

This Application Claims the benefit of U.S. Provisional Application No. 60/297,112 filed Jun. 8, 2001; United States Provisional Patent Application entitled “A Mouse Unable To Express Functional Alpha-4 Integrin Protein, And Methods For Assaying Compounds Or Agents For Alpha-4 Integrin Protein Antagonist Activity And A Genetic Marker For Evaluating Efficacy Of Modulators Of Signaling Activity Of A Vla-4 Receptor” filed on May 23, 2002, serial number unassigned, and United States Provisional Application entitled “A Mouse Unable To Express Functional Alpha-4 Integrin Protein, And Methods For Assaying Compounds Or Agents For Alpha-4 Integrin Protein Antagonist Activity And A Genetic Marker For Evaluating Efficacy Of Modulators Of Signaling Activity Of A Vla-4 Receptor” filed on May 29, 2002, serial number unassigned, wherein said Applications are hereby incorporated by reference in their entireties.

### PRIORITY CLAIM

This Application Claims benefit of British Provisional Application number 0124895.4 filed on Oct. 17, 2001, which is hereby incorporated by reference in its entirety.

### FIELD OF THE INVENTION

The present invention relates to a novel, useful, and heretofore unknown mouse that is unable to express functional alpha-4 integrin protein. The present invention also involves methods that utilize a mouse that is unable to express functional alpha-4 integrin protein, as well as information obtained therefrom, for assaying compounds or agents that modulate alpha-4 integrin protein activity, as well as for compounds or agents that modulate signaling activity of VLA-4 receptor.

### BACKGROUND OF THE INVENTION

Leukocytes are the main actors in the body's defense system against invading microorganisms. They also play the main role in attacking the body's own cells in autoimmune response processes and inflammation. Other cells that are part of the body's defense system are granulocytes and macrophages. These cells are non-specific components. Granulocytes consist of neutrophils, basophils and eosinophils, all of which can all release cytotoxic compounds upon encountering microorganisms. Macrophages can also kill intruding antigens by phagocytosis.

Furthermore, the lymphoid system, which is responsible for the antigen-specific immune response, consists of T and B cells, which are named after their origin or site of differentiation, respectively. In particular, T-cells are named after the thymus, the place where their main differentiation takes place. Three different types of T-cells exist. T-killer cells destroy cells that represent a foreign antigen, such as virus-infected cells. T-helper cells help B-cells to produce antibodies. The remaining type of T-cell is the T-suppressor cell, which mediate suppression of the humoral and cell-mediated branches of the immune system.

Likewise, B-cells are also named after the location in the body in which they differentiate and mature, i.e., bone marrow. Upon binding to a T-helper cell, the B-cell releases specific antibodies against a particular foreign antigen. The release of these antibodies leads to the destruction of the antigen.

All cells of the immune system, including those discussed above, circulate throughout the circulatory and lymphatic systems to protect the body from foreign antigens. Upon a foreign organism's invasion of the body, a variety of cascades and mechanisms within the immune system are activated to destroy the antigen. Sometimes however, the immune system attacks healthy autologous cells, or overreacts to the presence of an antigen, which results in diseases such as Asthma Bronchiale, Juvenile Diabetes, Morbus Basedow, or autoimmune diseases such as Hashimoto's thyroiditis, pernicious anemia, Addison's disease, diabetes mellitus, rheumatoid arthritis, systemic lupus erythematosus, dermatomyositis, Sjogren's syndrome, dermatomyositis, lupus erythematosus, multiple sclerosis, myasthenia gravis, Reiter's syndrome, or Graves disease, to name only a few.

The Alpha-4 Integrin Protein

Integrins form a large family of homologous transmembrane linker proteins. They act as mediators for cell-cell interactions, as well as cell-extracellular matrix interactions. All the receptors are heterodimers, that comprise an alpha and a beta chain that are non-covalently linked together. Those chains are transmembrane glycoprotein subunits. Presently, 16 different alpha and 8 different beta chains are known, and are combined to form at least 22 different integrins [Newham and Humphries, 1996]. The binding of an integrin protein to its ligand is dependent upon divalent cations such as Mg²⁺ or Ca²⁺.

The α-4 integrin protein forms either the VLA-4 (Very Late Antigen-4) receptor with a β-1, or the LPAM-1 (Lymphocytes Payer's Patch Adhesion Molecule) receptor with a β-7 chain. Both receptors are predominantly expressed on leukocytes of all subclasses, with the exception of the neutrophils [Bochner et al., 1991], [Dunon et al., 1996]. Recent studies suggest however that the alpha-4 integrin is also expressed on neutrophils [Issekutz, 1998], [Taooka et al., 1999]. The VLA-4 receptor is also expressed in various other tissues during development, such as muscle [Yang et al., 1996], [Rosen et al. 1992], liver, [Jaspers et al., 1995] placenta, and heart [Yang et al., 1995]. α4 integrins also bind to an alternatively spliced segment of fibronectin [Hynes, 1992], as a component of the extracellular matrix through the connecting segment-1 (CS-1). VLA-4 as well as LPAM-1 can also bind to VCAM-1 (vascular cell adhesion molecule) located on the endothelium. However, only LPAM-1 binds to MadCAM-1 (mucosal vascular addressin), which is located in the gut associated lymph nodes (Payer's Patches).

The VLA-4 Receptor and its Role in Adhesion

The VLA-4 receptor is constitutively expressed at a very low level on leukocytes [Chen et al., 1999], [Yednock et al., 1995], [Chan et al., 1991], [Shimizu et al., 1990]. The expression is rapidly upregulated upon activation of the cell via an “inside out” or “outside in“ mechanism. The VLA-4 receptor plays a key role in the firm adhesion of the leukocyte to the endothelium.

Adhesion of leukocytes to the endothelial membrane and transmigration into the tissue is a multi-step process, which involves a multitude of molecules. In general, extravasation of the leukocyte happens predominantly in the high endothelial venules (HEV), which are a specialized endothelium for lymphocyte migration, and are found in all secondary lymphoid organs with the exception of spleen [Girard and Springer, 1995]. Most recirculating lymphocytes selectively bind to HEV and do not firmly attach to other vascular endothelial cells. The recruitment of lymphocytes into the specific organs of the secondary lymphoid organs is referred to as “homing”.

Adhesion and migration of the lymphocytes in the HEVs is mediated initially through L-Selectin—sialyl Lewis X (sLeX) interactions, and after activation of the lymphocytes by LFA-1/ICAM-1 and LPAM-1/MadCAM-1 interactions. The factors and molecules involved in the activation are poorly characterized in the HEVs. In the peripheral non-lymphoid tissue, the first step of adhesion is tethering and rolling of the leukocytes along the endothelial membrane, which is mediated through Selectins. This interaction is transient unless additional adhesive pathways are activated.

Inflammation and the abundance of chemokines and cytokines in a particular area of the body leads to the activation of leukocytes. Moreover, inflammation also activates VLA-4, and its expression is upregulated through a variety of factors, such as the β-chemokine MIP-1β (macrophage inflammatory protein), which is presented to the leukocyte through binding to the adhesion molecule CD44. Upon presentation of MIP-1β to the leukocyte, VLA-4 is activated and tight cell adhesion is mediated. VLA-4 is also activated through IL-4 and TNF-α. Only after the firm adhesion to the endothelial membrane can the leukocyte migrate through the membrane and enter the tissue.

Efforts have been made to control inflammation via control of the activity of VLA-4. For example, blocking VLA-4 with specific antibodies have resulted in limited therapeutic effects. Moreover, it has been determined that blocking VLA-4 can inhibit extravasation of eosinophils through human umbilical vein endothelial cells (HUVEC) or eosinophil accumulation, and late asthmatic response in a guinea pig asthma-model [Sagara et al., 1997]. It has also been determined that blocking of VLA-4 with a soluble VCAM-Ig fusion protein can delay the onset of adoptively transferred autoimmune diabetes in non-obese diabetic mice [Jakubowski et al., 1995].

In addition, VLA-4 mediated adhesion, and thus migration of leukocytes into tissue, is proposed to play a major role in a variety of diseases such as Crescentic Nephritis [Allen et al, 1999], Rheumatoid Arthritis, Systemic Lupus Erythematosus, Diabetes Mellitus, Sjögren's Syndrome [McMurray 1996], Asthma, Multiple sclerosis and neurological disorders [Mousa and Cheresh, 1997].

In order to further understand the effects of VLA-4, or lack thereof in vivo, and to develop methods for assaying compounds or agents in vivo to determine whether agents have alpha-4 integrin antagonist activities, efforts have been made to develop mammals that are unable to express functional alpha-4 integrin protein. In particular, it is well understood that transgenic and knockout mice provide a valuable tool to determine gene or protein functions in vivo [Reviews by Capecchi, 1994, Capecchi, 1989, Capecchi, 1989]. Furthermore, knockout models have certain advantages over studies using antibodies, since antibodies can be potentially misleading due to artifacts that arise from inappropriate cross-linking events, Fc-receptor mediated effects, or inadequate penetration in vivo [Sharpe, 1995].

Knockout Mice of Adhesion Molecules

A homozygous knockout model has been developed for most of the adhesion molecules, and studied for cell-extracellular matrix interactions. (Reviewed by [Hynes, 1996], [George and Hynes, 1994], [Ley, 1995], [Hynes, 1994], [Hynes and Wagner, 1996], [Nebert and Duffy, 1997]). Several of the knockouts are lethal, among them the following integrins: alpha-4 [Yang et al. 1995], alpha-5 [Yang et al., 1993], alpha-6 [Georges-Labouesse et al., 1996], alpha-8 [mentioned in Hynes, 1996], alpha-9 [mentioned in Hynes, 1996], alpha-v [mentioned in Hynes, 1996], beta-1[Fässler and Meyer, 1995], [Stephens et al., 1995] and beta-4 [van der Neut et al., 1996].

Recent efforts to produce a homozygous alpha-4 integrin knockout have not been successful. It has been determined that the homozygous knockout of the α-4 integrin protein is embryonicaly lethal in mice due to severe defects in the developing placenta and heart. [Yang et al., 1995] In particular, it has been determined that in such a knockout mouse, the allantois fails to fuse with the chorion at day 11 during gestation. Although approximately 50% of the offspring overcome this failure, those remaining offspring die at day 14 during gestation due to failure of two layers of the developing heart to fuse together.

Accordingly, what is needed is a mouse that, although unable to produce functional alpha-4 integrin protein, can survive gestation and mature into a viable mouse. Such a mouse would have great utility in determining the genotypical and phenotypical effects that result in antagonizing functional alpha-4 integrin protein.

What is also needed are in vivo and in vitro methods of assaying compounds or agents for the ability to modulate alpha-4 integrin protein activity or the signaling activity of VLA-4 receptor, particularly antagonists thereof. Such compounds or agents have applications in treating inflammation, as well as a plethora of diseases and disorders related to the expression of alpha-4 integrin protein or VLA-4 receptor, including, but not limited to Asthma Bronchiale, Juvenile Diabetes, Morbus Basedow, or autoimmune diseases such as Hashimoto's thyroiditis, pernicious anemia, Addison's disease, diabetes mellitus, rheumatoid arthritis, systemic lupus erythematosus, dermatomyositis, Sjogren's syndrome, dermatomyositis, lupus erythematosus, multiple sclerosis, myasthenia gravis, Reiter's syndrome, or Graves disease, to name only a few.

The citation of any reference herein should not be construed as an admission that such reference is available as “Prior Art” to the instant application.

### SUMMARY OF THE INVENTION

In accordance with the present invention, there is provided a novel and useful mouse that is unable to produce functional alpha-4 integrin protein. In further accordance with the present invention, provided herein are novel in vivo and in vitro methods for assaying compounds or agents for their ability to modulate alpha-4 integrin protein activity or the signaling activity of VLA-4 receptor.

Thus broadly, the present invention extends to a mouse that is unable to express functional alpha-4 integrin protein.

Moreover, the present invention extends to a mouse that is unable to express functional alpha-4 integrin protein, wherein the mouse has a phenotype in which functional alpha-4 integrin protein can not be detected, and the level of a genetic marker measured in the mouse is modulated relative to the level of the genetic marker measured in a control wild type mouse. Particular genetic markers that are modulated in a mouse of the present invention relative to their levels measured in a control wild type mouse include, but certainly are not limited to:

Mus musculus anti-von Willebrand factor antibody NMC-4 kappa chain mRNA;

Mouse gene for immunoglobulin alpha heavy chain, switch region and con;

(H-2 class I histocompatibility antigen, d-k alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete cds;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

mt23g11.r1 Soares mouse 3NbMS Mus musculus cDNA clone 621956 5′ TIGR cds;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: poliovirus receptor homolog precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

Mus musculus Bop1 mRNA, complete cds;

C75959 Mouse 3.5-dpc blastocyst cDNA Mus musculus cDNA clone J0001C05;

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA CLASS II histocompatibility antigen, DO B;

NRNT(3e-39): Human phosphatidylinositol (4,5)bisphosphate 5-phosphatase;

Mus musculus (clone U2) T-cell specific protein mRNA, complete cds;

Mus musculus mRNA for peroxisomal integral membrane protein PMP34;

M. musculus mRNA for macrophage mannose receptor; and

the concentration of progenitor stem cells in blood,

to name only a few.

As explained infra, the measured levels of some of these genetic markers in a mouse of the present invention increase relative to the levels measured in a control wild type mouse, while the measured levels of other genetic markers in a mouse of the present invention decrease relative to the measured level of the genetic marker in a control wild type mouse. Genetic markers whose measured level in a mouse of the present invention increase relative to measured levels of these genetic markers in a control wild type mouse include:

Mus musculus anti-von Willebrand factor antibody NMC-4 kappa chain mRNA;

Mouse gene for immunoglobulin alpha heavy chain, switch region and con;

(H-2 CLASS I histocompatibility antigen, D-K alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete c;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: Poliovirus Receptor Homolog Precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

mt23g11.r1 Soares mouse 3NbMS Mus musculus cDNA clone 621956 5′ TIGR c;

Mus musculus Bop1 mRNA, complete cds;

C75959 Mouse 3.5-dpc blastocyst cDNA Mus musculus cDNA clone J0001C05; and

the concentration of progenitor stem cells in blood, to name only a few.

Likewise, examples of genetic markers whose measured levels in a mouse of the present invention decrease relative to the measured levels in a wild type control mouse include, but certainly are not limited to:

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA Class II histocompatibility antigen, DO B;

NRNT(3e-39): Human phosphatidylinositol (4,5)bisphosphate 5-phosphatase;

Mus musculus (clone U2) T-cell specific protein mRNA, complete cds; and

M. musculus mRNA for macrophage mannose receptor, to name only a few.

Furthermore, the present invention extends to a mouse that is unable to express functional alpha-4 integrin protein, wherein the mouse is a knockout mouse. Such a knockout mouse of the present invention comprises a first and second allele capable of expressing functional alpha-4 integrin protein, wherein the first allele comprises a defect that prevents the first allele from expressing functional alpha-4 integrin protein, and the second allele comprises a defect that prevents the second allele from expressing functional alpha-4 integrin protein. Such a knockout mouse also has within its genome two copies of a transgene comprising a portion of a cDNA molecule that encodes for alpha-4 integrin protein, operatively associated with a promoter. In a particular embodiment, the promoter used is the tetP promoter, and the transgene comprises a DNA sequence of SEQ ID NO: 1.

Numerous types of defects can be used to disrupt the expression of the alleles so that a knockout mouse of the present invention is unable to express functional alpha-4 integrin protein. Examples of such defects include, but certainly are not limited to, a substitution, insertion, and/or deletion of one or more nucleotides in the first allele and in the second allele.

Furthermore, the present invention extends to a method for making a knockout mouse that is unable to express functional alpha-4 integrin protein, comprising the steps of:

(a) crossing two knockout mice comprising a first and second allele capable of expressing functional alpha-4 integrin protein, wherein the knockout mice each comprise a defect in either the first allele or second allele, such that either the first or second allele in each knockout mouse is unable to express functional alpha-4 integrin protein;

(b) harvesting the embryos resulting from the cross of step (a),

(c) inserting a transgene comprising a portion of an isolated cDNA molecule that encodes for alpha-4 integrin protein operatively associated with a promoter, into each embryo harvested in step (b), to form a transfected embryo, so that the transgene is incorporated into the genome of the embryo;

(d) inserting the transfected embryo into a pseudopregnant female mouse so that the pseudopregnant female mouse gives birth to a mouse that comprises a first and second allele capable of expressing functional alpha-4 integrin protein, wherein either the first or the second allele comprises the defect that prevents the allele from expressing functional alpha-4 integrin protein, and the transgene; and

(e) crossing two mice produced in step (d) to produce a knockout mouse comprising a first and second allele capable of expressing functional alpha-4 integrin protein, wherein the first and the second alleles each comprise the defect that prevents the alleles from expressing functional alpha-4 integrin protein, and the genome of the knockout mouse comprises two copies of the transgene;

wherein the knockout mouse of step (e) is unable to express functional alpha-4 integrin protein.

Particular examples of heterozygous alpha-4 knockout mice having applications in a method of the present invention are heterozygous alpha-4 integrin knockout mice that can be readily obtained from Jackson Laboratory, Bar Harbor, Me., and have been assigned Jackson laboratory stock number 002463. These heterozygous knockout mice are described in detail infra.

In addition, the present invention extends to a method for making a knockout mouse that is unable to express functional alpha-4 integrin protein, wherein the transgene comprises a portion of an isolated cDNA molecule that encodes for alpha-4 integrin protein operatively associated with a tetP promoter, and has a DNA sequence of SEQ ID NO: 1.

Naturally, numerous methods of inserting the transgene into a mouse embryo are readily available to one of ordinary skill in the art. A particular method for such insertion comprises inserting the transgene into an expression vector, and then inserting the expression vector into the embryo. Other methods having applications herein are described infra.

Moreover, the present invention extends to a method for making a knockout mouse that is unable to express functional alpha-4 integrin protein, comprising the steps of:

(a) crossing two heterozygous alpha-4 integrin knockout mice assigned Jackson laboratories stock number 002463, wherein the two knockout mice have a defect in either the first or second allele that encode for functional alpha-4 integrin protein that disrupts the expression of functional alpha-4 integrin protein in one of the alleles;

(b) harvesting the embryos that result from the cross of step (a);

(c) inserting a transgene comprising a portion of a cDNA molecule that encodes an alpha-4 integrin protein operatively associated with a tetP promoter, and comprising a DNA sequence of SEQ ID NO: 1, into an embryo harvested in step (b), to form a transfected embryo, so that the genome of the embryo comprises the transgene;

(d) inserting the transfected embryo into a pseudopregnant female mouse so that the pseudopregnant female mouse gives birth to a heterozygous alpha-4 knockout mouse whose genome comprises the transgene; and

(e) crossing two mice produced in step (d) to produce a homozygous alpha-4 knockout mouse whose genome comprises two copies of the transgene.

The resulting knockout mouse is homozygous for the defect that disrupts ability of both alleles to expression of alpha-4 integrin protein, and contains within its genome two copies of the transgene. Thus, the resulting mouse survives gestation and matures into a viable mouse, but surprisingly and unexpectedly is unable to express functional alpha-4 integrin protein in the adult mouse.

In another embodiment, the present invention extends to a mouse that is unable to express functional alpha-4 integrin protein, wherein the mouse is a transgenic mouse. Such a transgenic mouse of the present invention has a genome in which the first and second alleles capable of expressing functional alpha-4 integrin protein have been replaced with two copies of a transgene that encodes for a non-functional truncated alpha-4 integrin protein, wherein the transgene comprises a portion of an isolated cDNA molecule that encodes for alpha-4 integrin protein, operatively associated with a promoter. As a result, a transgenic mouse of the present invention can survive gestation and mature into an adult mouse, but is unable to express functional alpha-4 integrin protein in the adult mouse.

Numerous methods are readily available to one of ordinary skill in the art to replace an endogenous allele or gene with a heterologous nucleic acid molecule. A particular method having applications herein is homologous recombination, which is described infra.

In a particular embodiment of a transgenic mouse of the present invention, the transgene comprises a portion of the cDNA molecule that encodes for alpha-4 integrin protein operatively associated with the tetP-promoter [Gossen and Bujard, 1992], and comprises a DNA sequence of SEQ ID NO: 1.

Naturally, the present invention further extends to a method for producing a transgenic mouse that is unable to express functional alpha-4 integrin protein. A first step in such a method comprises crossing a first transgenic mouse wherein either allele capable of expressing functional alpha-4 integrin protein is replaced with a transgene comprising a portion of an isolated cDNA molecule that encodes for the alpha-4 integrin protein operatively associated with a promoter, with a second transgenic mouse wherein either allele capable of expressing functional alpha-4 integrin protein is replaced with the transgene. The second step of such a method comprises selecting offspring from the cross that have a genome in which both alleles capable of expressing functional alpha-4 integrin protein have been replaced with two copies of the transgene. These selected offspring are transgenic mice of the present invention that are unable to express functional alpha-4 integrin protein in the adult mouse.

In addition, the present invention extends to methods for assaying compounds or agents for their ability to modulate, and particularly antagonize, alpha-4 integrin protein activity or signaling activity of VLA-4 receptor. In particular, the present invention extends to a method for assaying a compound or agent for the ability to modulate alpha-4 integrin protein activity of signaling activity of VLA-4 receptor, comprising the steps of (a) administering the compound or agent to mouse, (b) measuring the level of a genetic marker in the mouse, and (c) comparing the measurement of step (b) with the level of the genetic marker measured in a control mouse. Modulation of the level of the genetic marker measured in the treated mouse relative to the level of the genetic marker measured in the control mouse indicates the compound or agent may have efficacy as a modulator of alpha-4 integrin protein activity or signaling activity of VLA-4 receptor. Particular genetic markers having applications in such a method of the present invention include, but certainly are not limited to:

Mus musculus anti-von Willebrand factor antibody NMC-4 kappa chain mRNA;

Mouse gene for immunoglobulin alpha heavy chain, switch region and con;

(H-2 class I histocompatibility antigen, d-k alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete cds;

Mus musculus mRNA for erythroid differentiation regulator, partial;

NRNT(1e-92): , complete sequence [Mus musculus];

vc50e11.r1 Knowles Solter mouse 2 cell Mus musculus cDNA clone 778028;

mt23g11.r1 Soares mouse 3NbMS Mus musculus cDNA clone 621956 5′ TIGR cds;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: poliovirus receptor homolog precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

Mus musculus Bop1 mRNA, complete cds;

C75959 Mouse 3.5-dpc blastocyst cDNA Mus musculus cDNA clone J0001C05;

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA CLASS II histocompatibility antigen, DO B;

NRNT(3e-39): Human phosphatidylinositol (4,5)bisphosphate 5-phosphatase;

Mus musculus (clone U2) T-cell specific protein mRNA, complete cds;

Mus musculus mRNA for peroxisomal integral membrane protein PMP34;

M. musculus mRNA for macrophage mannose receptor; and

the concentration of progenitor stem cells in blood, to name only a few.

Furthermore, the present invention extends to a method of assaying compounds for their ability to modulate, and particularly to antagonize, alpha-4 integrin activity or signaling activity of VLA-4 receptor, wherein the modulation of the level of the genetic marker measured in the wild type mouse comprises an increase relative to the level of the genetic marker measured in the control wild type mouse. Examples of genetic markers that have been determined to have increased levels in a knockout mouse of the present invention relative to their levels in a wildtype mouse comprise:

Mus musculus anti-von Willebrand factor antibody NMC-4 kappa chain mRNA;

Mouse gene for immunoglobulin alpha heavy chain, switch region and con;

(H-2 CLASS I histocompatibility antigen, D-K alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete c;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: Poliovirus Receptor Homolog Precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

mt23g11.r1 Soares mouse 3NbMS Mus musculus cDNA clone 621956 5′ TIGR c;

Mus musculus Bop1 mRNA, complete cds;

C75959 Mouse 3.5-dpc blastocyst cDNA Mus musculus cDNA clone J0001C05; and

the concentration of progenitor stem cells in blood.

Hence, in a method of the present invention wherein the administration of a compound or agent to a mouse results in an increase in the levels of any of these genetic markers, the compound or agent is a potential antagonist of the activity of alpha-4 integrin protein activity of the signaling activity of VLA-4 receptor.

In addition, the present invention extends to a method of assaying compounds or agents for alpha-4 integrin antagonist activity, wherein the modulation of the level of the genetic marker measured in the wild type mouse comprises a decrease relative to the level of the genetic marker measured in the control wild type mouse. Examples of genetic markers whose levels decrease when the activity of alpha-4 integrin protein is decreased or antagonized comprise:

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA Class II histocompatibility antigen, DO B;

NRNT(3e-39): Human phosphatidylinositol (4,5)bisphosphate 5-phosphatase;

Mus musculus (clone U2) T-cell specific protein mRNA, complete cds; or

Mus musculus mRNA for macrophage mannose receptor.

Consequently, in a method of the present invention wherein the administration of a compound or agent to a mouse results in an decrease in the levels of any of these genetic markers, the compound or agent is a potential antagonist of the activity of alpha-4 integrin protein activity or the signaling activity of VLA-4 receptor.

Furthermore, the present invention extends to a method for assaying a compound or agent for activity in ameliorating deleterious side effects associated with an alpha-4 integrin protein antagonist, comprising the steps of:

(a) administering the compound or agent to a mouse of the present invention that is not able to express functional alpha-4 integrin protein;

(b) measuring the level of a genetic marker in the mouse; and

(c) comparing the level of the genetic marker measured in the mouse to the level of the genetic marker measured in a control mouse of the present invention that is not able to express functional alpha-4 integrin protein,

Modulation of the level of the genetic marker measured in the mouse to which the compound or agent was administered relative to the level of the genetic marker measured in the control mouse that is unable to express functional alpha-4 integrin protein indicates the compound or agent may have activity in ameliorating deleterious side effects associated with an alpha-4 integrin protein antagonist. Examples of genetic markers having applications in this embodiment are described above. Naturally, the modulation in this embodiment is in directions opposite to the modulation observed in a method for assaying a compound or agent for alpha-4 integrin antagonist activity, as described above. Thus, if a compound or agent having alpha-4 integrin activity upregulates a genetic marker, then a compound or agent that can ameliorate deleterious side effects of an alpha-4 integrin antagonist would downregulate that particular genetic marker.

Another property of a mouse of the present invention is that the concentration of progenitor stem cells in its blood is greater than the concentration of progenitor stem cells found in the blood of a mouse that is able to express functional alpha-4 integrin protein. This property of a mouse of the present invention can readily be used in an assay of compounds or agents for alpha-4 integrin protein antagonist activity. In particular, an increase in the concentration of progenitor stem cells measured in a mammal after the compound or agent is administered, relative to the concentration of progenitor cells measured in the blood of the mammal prior to administration of the compound or agent, is indicative of the compound's or agent's alpha-4 integrin protein antagonist activity, or antagonist activity to the signaling activity of VLA-4 receptor. Hence, the present invention extends to a method for assaying a compound or agent for potential alpha-4 integrin protein antagonist activity, comprising the steps of:

(a) removing a first blood sample from a mammal and measuring the concentration of progenitor stem cells in the first blood sample;

(b) administering the compound or agent to the mammal;

(c) removing a second blood sample from the mammal and measuring the concentration of progenitor stem cells in the second blood sample; and

(d) comparing the measured concentration of progenitor stem cells in the first blood sample with measured concentration of progenitor stem cells in the second blood sample.

An increase in the measured progenitor stem cell concentration in the second blood sample relative to the measured progenitor stem cell concentration in the first blood sample indicates the compound or agent may have alpha-4 integrin protein antagonist activity.

Moreover, the present invention extends to a method for assaying a compound or agent for alpha-4 integrin protein antagonist activity, comprising the steps of:

(a) administering the compound or agent to the mammal;

(b) measuring the concentration of progenitor stem cells in the blood of the mammal; and

(c) comparing the measured concentration of progenitor stem cells in the blood of the mammal to the measured concentration or progenitor stem cells in the blood of a control mammal,

wherein an increase in the concentration of progenitor stem cells in the blood of the mammal relative to the concentration of progenitor stem cells in the blood of the control mammal is indicative of potential alpha-4 integrin protein antagonist activity in the compound or agent.

Naturally, numerous types of mammals have application in a method of assaying a compound or agent for alpha-4 integrin antagonist activity. Particular examples include, but certainly are not limited to ovine, bovine, equine, canine, feline, murine, or human, to name only a few.

In another embodiment, the present invention extends to the use of a genetic marker described herein to have modulated levels in a knockout mouse of the present invention relative to its level in a wildtype mouse, to determine whether a compound or agent modulates signaling of the VLA-4 receptor. As explained above, the signaling of the VLA-4 receptor is dependent upon the activity of the alpha-4 integrin protein. Consequently, a modulation in alpha-4 integrin expression, such as a decrease, will result in a modulation of VLA-4 receptor signaling. Hence broadly, the present invention extends to method for determining whether a compound or agent modulates signaling of a VLA-4 receptor, comprising the steps of:

(a) administering the compound or agent to an organism;

(b) measuring the expression level of a genetic marker for VLA-4 receptor signaling in a bodily sample removed from the organism; and

(c) comparing the expression level of the genetic marker of step (b) with the expression level of the genetic marker measured in a control bodily sample,

wherein a difference between the measured expression level of the genetic marker in the bodily sample and the control bodily sample indicates that the compound or agent modulates the signaling of the VLA-4 receptor.

In a method of the present invention, a bodily sample includes, but certainly is not limited to a bodily fluid, e.g., blood, urine, saliva, mucus, semen, lymph, etc, or a solid sample such as tissue, bone, hair, etc. Naturally, the control bodily sample can be a bodily sample taken from the organism prior to the administration of the compound or agent, or alternatively, a bodily sample taken from a second organism substantially similar to the first organism (same or similar specie, age, weight, sex, etc.), to which the compound or agent is not administered.

Moreover, due to the direct relationship between the activity of alpha-4 integrin protein and the signaling activity of VLA-4 receptor, genetic markers that are modulated in a knockout mouse of the present invention that is unable to express functional alpha-4 integrin protein are also genetic markers for a modulation of the signaling activity of VLA-4 receptor. Examples of such genetic markers include, but certainly are not limited to:

(Mus musculus anti-von Willebrand factor antibody NMC-4 kappa chain mRNA;

Mouse gene for immunoglobulin alpha heavy chain, switch region and con;

(H-2 class I histocompatibility antigen, d-k alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete cds;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

mt23g11.r1 Soares mouse 3NbMS Mus musculus cDNA clone 621956 5′ TIGR cds;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: poliovirus receptor homolog precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

Mus musculus Bop1 mRNA, complete cds;

C75959 Mouse 3.5-dpc blastocyst cDNA Mus musculus cDNA clone J0001C05;

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA CLASS II histocompatibility antigen, DO B;

NRNT(3e-39): Human phosphatidylinositol (4,5)bisphosphate 5-phosphatase;

Mus musculus (clone U2) T-cell specific protein mRNA, complete cds;

Mus musculus mRNA for peroxisomal integral membrane protein PMP34;

M. musculus mRNA for macrophage mannose receptor; and

the concentration of progenitor stem cells in blood; to name only a few.

Examples of these genetic markers whose level of expression is increased in a knockout mouse of the present invention, and thus is increased when the signaling activity of VLA-4 receptor is antagonized, comprise:

Mus musculus anti-von Willebrand factor antibody NMC-4 kappa chain mRNA;

Mouse gene for immunoglobulin alpha heavy chain, switch region and con;

(H-2 CLASS I histocompatibility antigen, D-K alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete c;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: Poliovirus Receptor Homolog Precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

mt23g11.r1 Soares mouse 3NbMS Mus musculus cDNA clone 621956 5′ TIGR c;

Mus musculus Bop1 mRNA, complete cds;

C75959 Mouse 3.5-dpc blastocyst cDNA Mus musculus cDNA clone J0001C05; and

the concentration of progenitor stem cells in blood.

Hence, in a method of the present invention, wherein a compound or agent administered to an organism results in an increased level of any of these genetic markers relative to measurements in a control organism, the compound or agent is an antagonist of the signaling activity of VLA-4 receptor.

Examples of genetic markers for signaling activity of VLA-4 receptor that exhibit decreased levels when the signaling activity of VLA-4 receptor is antagonized comprise:

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA Class II histocompatibility antigen, DO B;

NRNT(3e-39): Human phosphatidylinositol (4,5)bisphosphate 5-phosphatase;

Mus musculus (clone U2) T-cell specific protein mRNA, complete ds; and

M. musculus mRNA for macrophage mannose receptor.

Thus, a compound or agent administered to an organism that results in a decrease in the levels of any of these genetic markers relative to levels measured in a control organism indicates that the compound or organism is a VLA-4 receptor antagonist.

In a particular embodiment, the present invention extends to a method for determining whether a compound or agent modulates signaling of the VLA-4 receptor, wherein the genetic marker is the M. musculus mRNA for macrophage mannose receptor whose nucleotide sequence has been assigned GenBank accession number Z11974, and is set forth in SEQ ID NO: 13. As explained above, if the administration of the compound or agent results in decreased levels of M. musculus mRNA for macrophage mannose receptor, the compound or agent is an antagonist of signaling activity of VLA-4 receptor, and may readily have applications as a medicament for treating diseases or disorders such as asthma, arthritis, MS and others.

Similarly, the present invention extends to a method for determining whether a compound or agent modulates signaling of the VLA-4 receptor, wherein the genetic marker comprises Mus musculus mRNA for JAB, complete cds, or SOCS-1 protein, whose nucleotide sequence has been assigned GenBank accession number AB000677, and is set forth in SEQ ID NO: 17, EST AA571535 having a DNA sequence of SEQ ID NO: 18 (FIG. 21) or EST AA154371 having a nucleotide sequence of SEQ ID NO: 21 (FIG. 23). If the administration of the compound or agent results in decreased levels of one of these genetic markers, the compound or agent is an antagonist of signaling activity of VLA-4 receptor, and may readily have applications as a medicament for treating diseases or disorders such as asthma, arthritis, MS and others.

Numerous types of compounds or agents have applications in a method of the present invention. Examples of such types include, but certainly are not limited to a protein; e.g., an antibody having a VLA-4 receptor as an immunogen, or a fragment of such a an antibody, or an antibody having alpha-4 integrin protein as an immunogen, or a fragment of such an antibody; a chemical compound; a nucleic acid molecule such as an antisense molecule that hybridizes to RNA encoding VLA-4 receptor or an alpha-4 integrin protein, or a ribozyme that cleaves RNA encoding a VLA-4 receptor or an alpha-4 integrin protein; a carbohydrate; or a hormone. Particular examples of a compound or agent having applications herein are set forth in U.S. Pat. Nos. 6,331,552, 6,352,977, and PCT published patent application WO99/23063, which are hereby incorporated by reference in their entireties.

In addition, the present invention extends to a method for determining the efficacy of a potential antagonist of the signaling of a VLA-4 receptor, wherein such a method comprises the steps of:

(a) removing a first bodily sample from an organism;

(b) measuring the level of M. musculus mRNA for macrophage mannose receptor genetic marker in the first bodily sample;

(c) administering the potential antagonist to the organism;

(d) removing a second bodily sample from the organism;

(e) measuring the level of the genetic M. musculus mRNA for macrophage mannose receptor genetic marker in the second bodily sample; and

(f) comparing the measured levels of step (b) and step (e).

A decrease in the measured level of the genetic marker in step (e) relative to the measured level of the genetic marker in step (b) indicates that the potential antagonist possesses efficacy in antagonizing the signaling activity of VLA-4. Naturally, as described above, the first and second bodily samples may comprise a bodily fluid, a bodily tissue, or a combination thereof. The nucleotide sequence of the genetic marker M. musculus mRNA for macrophage mannose receptor has been assigned Accession number: Z11974, and is set forth in SEQ ID NO: 13.

The present invention further extends to a method for determining the efficacy of a potential antagonist of the signaling of a VLA-4 receptor, wherein such a method comprises the steps of:

(a) removing a first bodily sample from an organism;

(b) measuring the level of Mus musculus mRNA for JAB genetic marker in the first bodily sample;

(c) administering the potential antagonist to the organism;

(d) removing a second bodily sample from the organism;

(e) measuring the level of the M. musculus mRNA for JAB genetic marker in the second bodily sample; and

(f) comparing the measured levels of step (b) and step (e).

A decrease in the measured level of the genetic marker in step (e) relative to the measured level of the genetic marker in step (b) indicates that the potential antagonist possesses efficacy in antagonizing the signaling activity of VLA-4. Naturally, as described above, the first and second bodily samples may comprise a bodily fluid, a bodily tissue, or a combination thereof. The nucleotide sequence of the genetic marker Mus musculus mRNA for JAB, complete cds, or SOCS-1 protein, has been assigned GenBank accession number AB000677 and is set forth in SEQ ID NO: 17.

In addition, the present invention further extends to a method for determining the efficacy of a potential antagonist of the signaling of a VLA-4 receptor, wherein such a method comprises the steps of:

(a) removing a first bodily sample from an organism;

(b) measuring the level of EST AA571353 genetic marker in the first bodily sample;

(c) administering the potential antagonist to the organism;

(d) removing a second bodily sample from the organism;

(e) measuring the level of the EST AA571353 genetic marker in the second bodily sample; and

(f) comparing the measured levels of step (b) and step (e).

A decrease in the measured level of the genetic marker in step (e) relative to the measured level of the genetic marker in step (b) indicates that the potential antagonist possesses efficacy in antagonizing the signaling activity of VLA-4. Naturally, as described above, the first and second bodily samples may comprise a bodily fluid, a bodily tissue, or a combination thereof. The nucleotide sequence of the genetic marker EST AA571353 (vm06f11.r1 Knowles Solter mouse blastocyst B1 Mus musculus cDNA clone), is set forth in SEQ ID NO: 21.

Moreover, the present invention extends to a method for determining the efficacy of a potential antagonist of the signaling of a VLA-4 receptor, wherein such a method comprises the steps of:

(a) removing a first bodily sample from an organism;

(b) measuring the level of EST AA154371 genetic marker in the first bodily sample;

(c) administering the potential antagonist to the organism;

(d) removing a second bodily sample from the organism;

(e) measuring the level of the EST AA571353 genetic marker in the second bodily sample; and

(f) comparing the measured levels of step (b) and step (e).

A decrease in the measured level of the genetic marker in step (e) relative to the measured level of the genetic marker in step (b) indicates that the potential antagonist possesses efficacy in antagonizing the signaling activity of VLA-4. Naturally, as described above, the first and second bodily samples may comprise a bodily fluid, a bodily tissue, or a combination thereof. The nucleotide sequence of the genetic marker EST AA154371 (Homologous to sp P13765: HLA CLASS II histocompatibility antigen, DO B), is set forth in SEQ ID NO: 23.

As explained above, numerous types of organisms have application in a method of assaying the efficacy of a potential antagonist of the signaling of a VLA-4 receptor. Particular examples include, but certainly are not limited mammals such as ovine, bovine, equine, canine, feline, murine, or human, to name only a few.

Moreover, assaying compounds or agents for their ability to modulate signaling activity of VLA-4 receptor, and particularly antagonizing such activity, can also be performed with in vitro methods of the present invention. Hence, the present invention further extends to a method for determining the ability of a compound or agent to modulate, and particularly to antagonize, the signaling activity of VLA-4 receptor, comprising the steps of:

(a) contacting the compound or agent with a bodily sample from an organism;

(b) measuring the expression level of a genetic marker for VLA-4 receptor signaling in the bodily sample; and

(c) comparing the expression level of the genetic marker measured in step (b) with the expression level of the genetic marker measured in a control bodily sample.

If the level increases of a VLA-4 receptor marker that has been found to exhibit increased levels when the signaling activity of VLA-4 receptor is antagonized, then the compound or agent is an antagonist of the signaling activity of VLA-4 receptor. Similarly, if the level decreases of a VLA-4 receptor marker that has been found to exhibit decreased levels when the signaling activity of VLA-4 receptor is antagonized, then the compound or agent is an antagonist of the signaling activity of VLA-4 receptor. Examples of VLA-4 genetic markers, and the modulation as a result of decreased signaling activity of VLA-4 receptor are discussed above.

Accordingly it is an object of the present invention to provide a mouse that is unable to express functional alpha-4 integrin protein.

It is another object of the present invention to provide methods for producing a mouse that is unable to express functional alpha-4 integrin protein.

It is another object of the present invention to provide useful and heretofore unknown methods of assaying compounds or agents for their ability of modulate alpha-4 integrin activity or signaling activity of VLA-4 receptor.

It is yet another object of the present invention to provide useful, novel, and heretofore unknown in vivo methods of assaying compounds or agents for alpha-4 integrin antagonist activity that utilize phenotypic information obtained from a mouse of the present invention that is unable to express functional alpha-4 integrin protein.

It is yet still another embodiment of the present invention to provide novel and useful methods for determining whether a compound or agent modulates signaling of a VLA-4 receptor.

These and other aspects of the present invention will be better appreciated by reference to the following drawings and Detailed Description.

### BRIEF DESCRIPTION OF THE DRAWINGS

FIG. 1: nucleotide sequence of SEQ ID NO: 1.

FIG. 2: Schematic map of the alpha-4 integrin cDNA. Locations of the primers used for cloning are indicated (fV and cDNA1B-R for the first part and cDNA2-F and cDNA2-R for the second part of the cDNA). The start and stop codons and the resulting open reading frame (ORF) are indicated as well as restriction sites relevant for the cloning of the cDNA into the vectors. The gene has four polyadenylation sites, all located 3′ of cDNA2-R [De Meirsman et al., 1996].

FIG. 3: Plasmid pBSK 2.6.

FIG. 4: Plasmid pCR 2cDNA.

FIG. 5: Plasmid pNEB 1.1.

FIG. 6: Plasmid pNEB 1.1(−).

FIG. 7: Typical pedigree of a heterozygous female x homozygous male cross: only one mouse out of 9 pups total offspring is a homozygous knockout, all others are heterozygous knockouts. Squares indicate male and circles female animals. Symbols with dots in the middle show homozygous knockout mice, symbols half black, half white show heterozygous knockout mice. The first litter was born Aug. 24, 1999 and the second litter was born Sep. 13, 1999.

FIG. 8: PCR analysis to assess the endogenous alpha-4 integrin background. The location of the primers are indicated in the schematic map of the genomic alpha-4 integrin DNA in FIG. 9B

FIG. 9 Genotype analysis of the alpha-4 integrin background

A) Southern blot of genomic tail DNA cut with PstI and hybridized with a 1.4 kb PstI/KpnI probe. Two bands of about 3 and 3.5 kb indicate a heterozygous mouse, only one band of 3.5 kb indicates a mouse with a homozygous KO for the endogenous alpha-4 integrin.

B) Schematic map of the genomic alpha-4 integrin DNA. Location of the 1.4 kb probe as well as the replaced area for the generation of the alpha-4 knockout mice by Yang et al. are indicated. Approximate locations of the primers used for PCR are indicated. Only relevant restriction sites are shown.

FIG. 10: Immuno histochemistry (IHC) analysis results for spleen sections and stained with a specific alpha-4 integrin antibody (CD49d, clone 9C10, BD Pharmingen). The brown staining in the wt mouse indicates the alpha-4 integrin protein, which is absent in the KO mouse.

FIG. 11: Schematic map of the tep-VLA transgene (the transgene comprising a portion of alpha-4 integrin operatively associated with a tetP promoter, and having a DNA sequence of SEQ ID NO: 1), with the location of the probe and indication of the protected fragments for both the endogenous RNA and transgenic RNA. For the RNA protection assays, typically 20-30 μg of DNase treated total RNA were used. The probe consisted of about 60 bp of tet-promoter 3′ of the transcriptional start site and ends in the second exon of the alpha-4 DNA, thus hybridizing to both endogenous as well as transgenic RNA, and leading to different sizes of protected RNA. The protected size for the transgenic RNA is about 550 nt, where as the size of the protected endogenous RNA is 490 nt. Linearized probe has the size of about 700 nt.

FIG. 12: RNase protection assay (RPA) of homozygous alpha-4 integrin knockout (KO) mice of the present invention (line 59) in comparison to FVB mice. The protected size of the transgene for the tissue samples from mice of line 59 are significantly shorter than expected.

FIG. 13: RPA of KO mice of two different lines, thymus and spleen. The transgene size of line 2 is of expected size, whereas line 1 shows the truncated form of the transgene. The line showing the correct size however has much lower transgene-levels in comparison to the endogenous expression levels as shown in the FVB mice. FVB and C57 (wild type) mice show the band of the protected endogenous RNA.

FIG. 14: plasmid pNEB 3.7 (−). This plasmid contains the full alpha-4 cDNA of about 3.6 kb.

FIG. 15: The tetP-VLA transgene. The alpha-4 cDNA is driven by the tet-promoter. Prior to microinjection, this plasmid was linearized with XhoI.

FIG. 16: M. musculus mRNA for macrophage mannose receptor Results Taqman analysis. The mRNA levels of the HMR 1031 and IVL 984 treated EAE mice in the brain samples are statistically significant lower in comparison to the vehicle controlled mice. The spleen samples do not show the same results as brain in either treatment. (*: p-value: 001 to 0.05).

FIG. 17: nucleotide sequence of SEQ ID NO: 13

FIG. 18: Clinical assessment of the EAE mice used for Taqman analysis.

FIG. 19: nucleotide sequence of SEQ ID NO: 17

FIG. 20: JAB FACS results.

FIG. 21: nucleotide sequence of SEQ ID NO: 18.

FIG. 22: Detailed Taqman results from Example IV using EST AA571535 as a genetic marker.

FIG. 23: nucleotide sequence of SEQ ID NO: 21.

FIG. 24: Detailed Taqman results from Example V using EST AA154371 as a genetic marker.

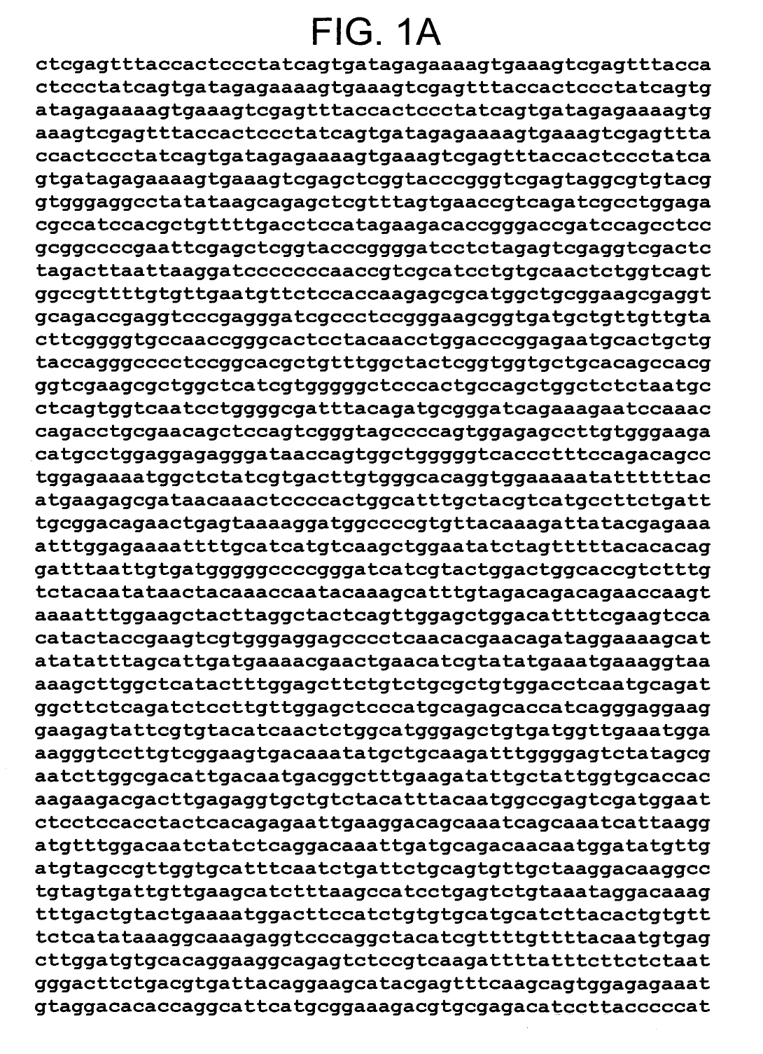

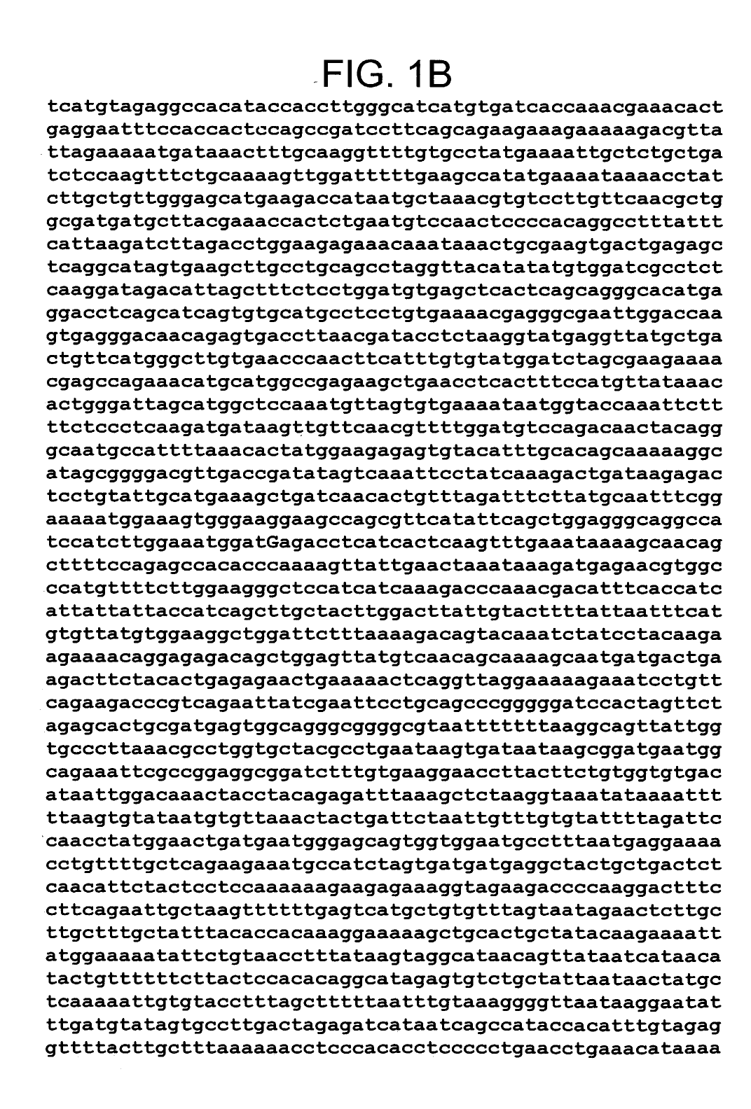

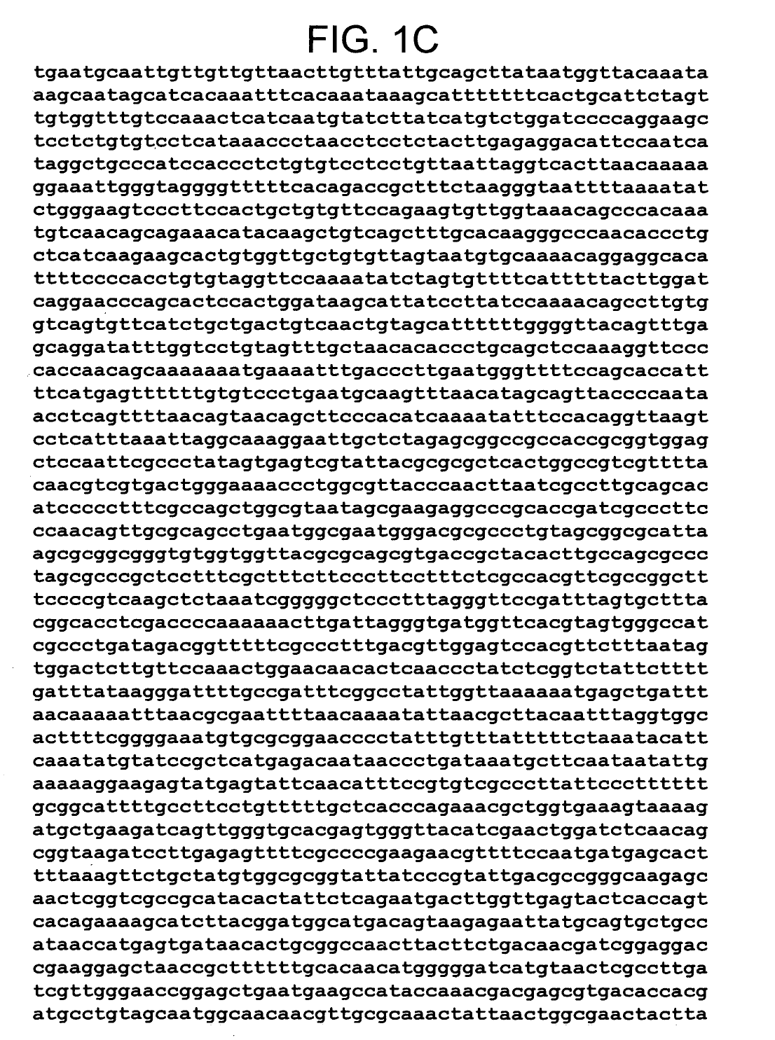

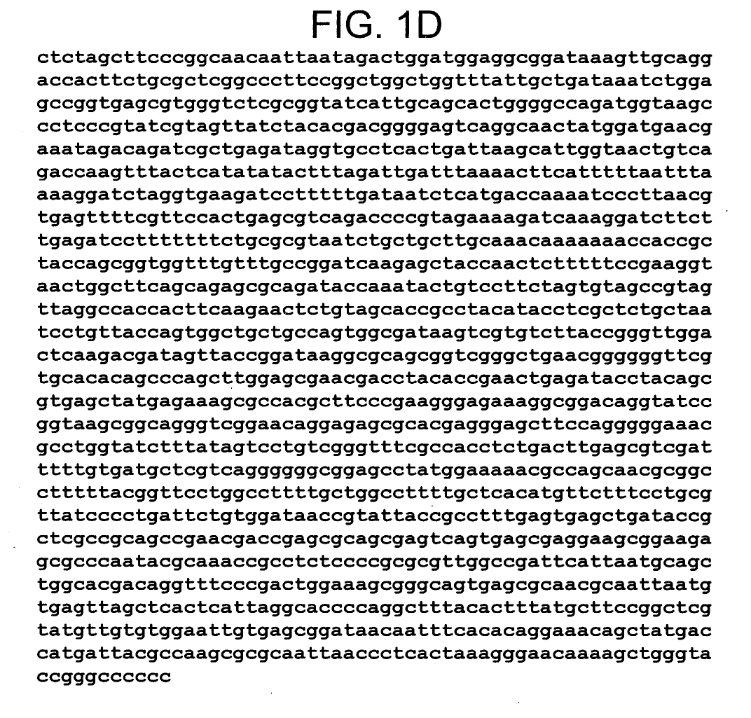

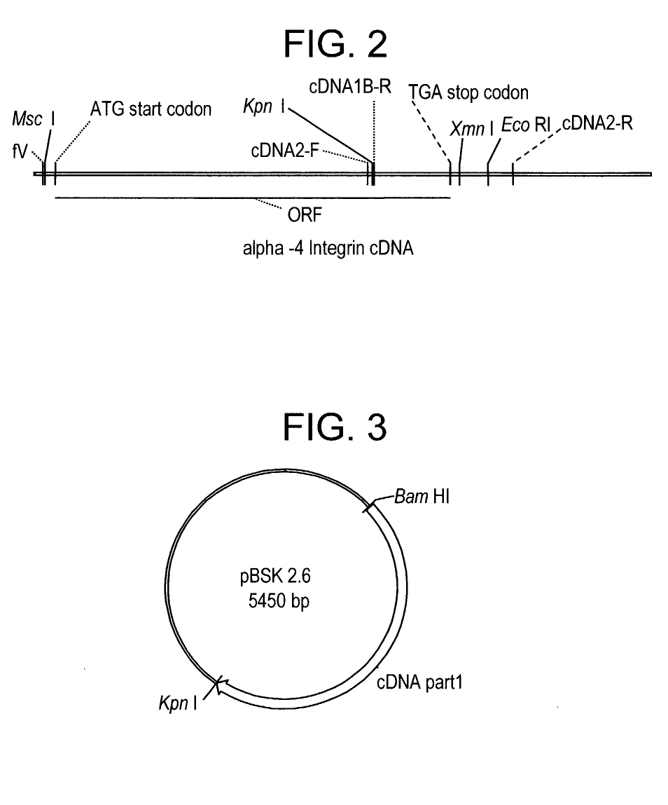

### DETAILED DESCRIPTION OF THE INVENTION

The present invention is based upon the discovery that a mouse can be successfully made that is unable to express functional alpha-4 integrin protein. Such a mouse has ready applications in methods for assaying compounds or agents for alpha-4 integrin antagonist activity.

Moreover, a mouse of the present invention that is unable express functional alpha-4 integrin protein possesses a heretofore unknown phenotype. In particular, levels of particular genetic markers measured in the mouse are modulated with respect to measured levels of these same genetic markers in a wild type mouse. This phenotypical data has great utility in methods to assay compounds or agents for alpha-4 integrin antagonist activity.

Thus broadly, the present invention extends to a mouse that is unable to express functional alpha-4 integrin activity. Examples of such a mouse include, but certainly not limited to, a knockout mouse and transgenic mouse, both of which are described infra.

It is noted that numerous terms and phases are used throughout the instant specification and claims. Definitions of these terms and phrases are provided below:

As used herein, the term “transgenic” describes a plant or animal that has stably incorporated one or more isolated nucleic acid molecules that encode for a protein or polypeptide, or protein, and can pass them on to successive generations.

As used herein, the term “knockout” refers to a mouse in which the expression of a particular gene in the genome of the mouse is disrupted.

As used herein, the term “modulation” refers to a change in the measured levels of a genetic marker as compared to a control. This modulation can be an increase in the measured level of the genetic marker relative to the measured level of the genetic marker in a control. Alternatively, the modulation can be a decrease in the measured level of a genetic marker relative to measured level of the genetic marker in the control.

As used herein, the term “portion” of an isolated nucleic acid molecule that encodes for a particular protein, refers to a part or fragment of the isolated nucleic acid molecule that comprises a sufficient number of contiguous nucleotides that encode for a peptide or polypeptide. Naturally, a “portion” of an isolated nucleic acid molecule is greater than one nucleotide, and the peptide or polypeptide encoded by the portion contains numerous amino acid residues, as described in the definitions of peptide and polypeptide below.

As used herein, the term “peptide” refers to two or more amino acids covalently joined by peptide binds. In a particular embodiment, a peptide comprises at least 10, preferably at least 20, more preferably at least 30, even more preferably at least 40, and most preferably 50 or more amino acids.

As used herein, the term “polypeptide” refers to a linear polymer composed of multiple contiguous amino acids. In particular, a polypeptide may possess a molecular weight greater than 100 kD.

As used herein, the term “phenotype” refers to the observable character of a cell or an organism. Such observable character can involve the physical appearance, as well as a level of particular physiological compositions present in the cell or organism.

As used herein, the term “control” with respect to an organism or a bodily sample of an organism refers to the organism or a bodily sample taken from the organism prior to the administration of the compound or agent, or alternatively, a second organism substantially similar to the first organism (same or similar specie, age, weight, sex, etc.) to which the compound or agent is not administered, or a second bodily sample taken from a second organism substantially similar to the first organism (same or similar specie, age, weight, sex, etc.), to which the compound or agent is not administered. Thus, a “control” is untreated with the compound or agent being assayed.

As used herein, the term “genetic marker” refers to a physiological composition whose measured RNA or protein level within an organism serves to identify whether a particular protein is present or functional within the organism. Moreover, a genetic marker may encode the particular protein or alternatively, may serve as a “surrogate” marker for a protein whose activity is related to the level of the genetic marker in a bodily sample. This relationship may be direct, wherein a decrease in the level of protein activity corresponds to a decrease in the level of the genetic marker, or alternatively, the relationship may be inverse, wherein a decrease in the level of protein activity corresponds to an increase in the level of the genetic marker. Such physiological compositions include, but certainly are not limited to, cells (e.g., progenitor stem cells) proteins, polypeptides, DNA, RNA, carbohydrates, or fatty acids, to name only a few. In a particular embodiment of the present invention, the measured levels of certain genetic markers are modulated in a mouse of the present invention with respect to the measured of levels of such genetic markers in a wild type control mouse. Examples of such genetic markers whose measured levels are modulated in a mouse of the present invention relative to levels measured in a wild type mouse include, but certainly are not limited to:

Mus musculus anti-von Willebrand factor antibody NMC-4 kappa chain mRNA;

Mouse gene for immunoglobulin alpha heavy chain, switch region and con;

(H-2 class I histocompatibility antigen, d-k alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete cds;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

mt23g11.r1 Soares mouse 3NbMS Mus musculus cDNA clone 621956 5′ TIGR cds;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: poliovirus receptor homolog precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

Mus musculus Bop1 mRNA, complete cds;

C75959 Mouse 3.5-dpc blastocyst cDNA Mus musculus cDNA clone J0001C05;

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA CLASS II histocompatibility antigen, DO B;

NRNT(3e-39): Human phosphatidylinositol (4,5)bisphosphate 5-phosphatase;

Mus musculus (clone U2) T-cell specific protein mRNA, complete cds;

Mus musculus mRNA for peroxisomal integral membrane protein PMP34;

M. musculus mRNA for macrophage mannose receptor; and

the concentration of progenitor stem cells in blood.

Examples of genetic markers for alpha-4 integrin or signaling activity of VLA-4 receptor that have a direct relationship with the activity of either of these proteins include, but certainly are not limited to:

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA Class II histocompatibility antigen, DO B;

NRNT(3e-39): Human phosphatidylinositol (4,5)bisphosphate 5-phosphatase;

Mus musculus (clone U2) T-cell specific protein mRNA, complete cds; and

M. musculus mRNA for macrophage mannose receptor, to name only a few.

Similarly, examples of genetic markers having an inverse relationship with the activity of alpha-4 integrin protein or the signaling activity of VLA-4 receptor, and thus exhibit increase levels when the activity of either of these proteins is decreased comprises:

Mouse gene for immunoglobulin alpha heavy chain, switch region and con;

Mus musculus anti-von Wllebrand factor antibody NMC-4 kappa chain, mRNA;

(H-2 CLASS I histocompatibility antigen, D-K alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete cds;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: Poliovirus Receptor Homolog Precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

mt23g11.r1 Soares mouse 3NbMS Mus musculus cDNA clone 621956 5′ TIGR c;

Mus musculus Bop1 mRNA, complete cds;

C75959 Mouse 3.5-dpc blastocyst cDNA Mus musculus cDNA clone J0001C05; and

the concentration of progenitor stem cells in blood.

A particular example of a “surrogate genetic marker” for the signaling activity of VLA-4 receptor is M. musculus mRNA for macrophage mannose receptor, whose nucleic acid sequence has been assigned GenBank Accession number: Z11974, and is set forth in SEQ ID NO: 13. Consequently, a compound or agent administered to an organism that reduces the measured level of macrophage mannose receptor mRNA demonstrates an ability to antagonize the signaling activity of VLA-4 receptor.

Another particular example of a “surrogate genetic marker” for the signaling activity of VLA-4 receptor is M. musculus mRNA for JAB, complete cds, whose nucleic acid sequence has been assigned GenBank Accession number AB AB000677, and is set forth in SEQ ID NO: 17. Consequently, a compound or agent administered to an organism that reduces the measured level of M. musculus mRNA for JAB, complete cds, demonstrates an ability to antagonize the signaling activity of VLA-4 receptor.

Other particular examples of a “surrogate genetic marker” for the signaling activity of VLA-4 receptor are EST AA571535 (vm06f11.r1 Knowles Solter mouse blastocyst B1 Mus musculus cDNA clone), having a nucleotide sequence of SEQ ID NO: 21, and EST AA154371 (Homologous to sp P13765: HLA CLASS II histocompatibility antigen, DO B) having a nucleotide sequence of SEQ ID NO: 23.

As used herein, the term “allele” refers to one of a set of alternative forms of a gene. In a diploid cell, each gene will have two alleles, each occupying the same position (locus) on homologous chromosomes.

As used herein, the term “pseudopregnant” refers to an anestrous state resembling pregnancy that occurs in various mammals usually after an infertile copulation.

As used herein, the term “wild type” refers to the normal, non-mutant form of an organism, i.e., the form found in nature.

As used herein, the phrase “progenitor stem cell” refers to relatively undifferentiated cells found in blood that have lost the capacity for self-renewal and are committed to a given cell lineage. For example, the myeloid stem cell generates progenitor stem cells for red blood cells (erythrocytes), the various white blood cells (neutrophils, eosinophils, basophils, monocytes, mast cells) and platelets. In a mouse of the present invention, the concentration of such progenitor stem cells in its blood is greater than the concentration of such progenitor stem cells in the blood of a wild type control mouse.

As used herein the terms “transgene” and “transgenic DNA” can be used interchangeably, and refer to an exogenous isolated nucleic acid molecule that is being inserted into the genome of a murine embryo for use in the production of a mouse of the present invention. In a particular embodiment, the transgene comprises a portion of a cDNA molecule that encodes alpha-4 integrin, operatively associated with the tetP promoter. More particularly, a transgene comprises a DNA sequence of SEQ ID NO: 1.

As used herein, the terms “compound” or “agent” refer to any composition presently known or subsequently discovered. Examples of compounds or agents having applications herein include organic compounds (e.g., man made, naturally occurring and optically active), peptides (man made, naturally occurring, and optically active, i.e., either D or L amino acids), carbohydrates, nucleic acid molecules, etc.

As used herein, the term “heterozygous” refers to an organism having two different alleles of a specified gene. More particularly, a knockout mouse that is “heterozygous” with respect to a particular protein is a mouse whose genome possesses an allele that is capable of expressing the protein, and an allele that normally is capable of expressing the protein, but possesses a defect that disrupts the successful expression of the protein. Thus, a heterozygous knockout mouse is capable of expressing the particular protein.

As used herein, the term “homozygous” refers to an organism having two identical alleles of a specified gene. More particularly, a knockout mouse that is “homozygous” with respect to a particular protein is a mouse whose genome possesses two alleles that normally are capable of expressing the protein, but each allele possesses a defect that disrupts the successful expression of the protein. Thus, a homozygous knockout mouse is unable to express the particular protein.

Furthermore, in accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Therefore, if appearing herein, the following terms shall have the definitions set out below.

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Heterologous” DNA refers to DNA not naturally located in the cell, or in a chromosomal site of the cell. In particular, the heterologous DNA includes a gene foreign to the cell.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

“Homologous recombination” refers to the insertion of a foreign DNA sequence of a vector into a chromosome at a specific chromosomal site. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A DNA “coding sequence” is a double-stranded DNA sequence that is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ of the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” or “promoter” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A DNA sequence is “operatively associated” to an expression control sequence when the expression control sequence controls and regulates the transcription and translation of that DNA sequence. The term “operatively associated” includes comprising an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

A coding sequence is “under the control” of transcriptional and translational control sequences in a cell when RNA polymerase transcribes the coding sequence into mRNA, which is then trans-RNA spliced and translated into the protein encoded by the coding sequence.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

As used herein, the term “sequence homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50:667).

Accordingly, the term “sequence similarity” in all its grammatical forms refers to the degree of identity or correspondence between nucleic acid or amino acid sequences of proteins that do not share a common evolutionary origin (see Reeck et al., supra). However, in common usage and in the instant application, the term “homologous,” when modified with an adverb such as “highly,” may refer to sequence similarity and not a common evolutionary origin.

In a specific embodiment, two nucleic acid sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Similarly, in a particular embodiment, two amino acid sequences are “substantially homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program, using default parameters.

The term “corresponding to” is used herein to refer similar or homologous sequences, whether the exact position is identical or different from the molecule to which the similarity or homology is measured. Thus, the term “corresponding to” refers to the sequence similarity, and not the numbering of the amino acid residues or nucleotide bases.

Hence, in a clinical setting wherein VLA-4 antagonists are being tested in humans, one of ordinary skill in the art need only assay the expression of a human gene that corresponds to or is homologous to one of the genetic markers described herein in order to evaluate the efficacy of the compound being tested. As explain above, this homology or correspondence can be readily determined by one of ordinary skill in the art using routine laboratory techniques.

Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990). In a particular embodiment of the present invention, the “host cell” is a murine embryo.

A Knockout Mouse that is Unable to Express Functional Alpha-4 Integrin Protein

As explained above, transgenic and knockout mice provide valuable tools to determine gene or protein functions in vivo [Reviews by Capecchi, 1994, Capecchi, 1989, Capecchi, 1989]. Furthermore, knockout models have certain advantages over studies using antibodies, since antibodies can be potentially misleading because of artifacts arising from inappropriate cross-linking events, Fc-receptor mediated effects and inadequate penetration in vivo [Sharpe, 1995].

The present invention extends to a mouse that is unable to express functional alpha-4 integrin protein, wherein the mouse is a knockout mouse. In a knockout mouse of the present invention, both alleles that are capable of expressing alpha-4 integrin protein are disrupted so that they are unable to express functional alpha-4 integrin protein, and two copies of a transgene comprising a portion of the cDNA molecule that encodes alpha-4 integrin protein operatively associated with a promoter, are inserted into the genome of the knockout mouse. As a result, a knockout mouse of the present invention survives gestation to mature into a mouse, but the adult mouse is unable to express functional alpha-4 integrin protein. In a particular embodiment of the present invention, the transgene comprises a portion of a cDNA molecule that encodes alpha-4 integrin protein, operatively associated with a tetP promoter, and has a DNA sequence of SEQ ID NO: 1.

Furthermore, the present invention extends to a method of making a knockout mouse that is unable to express functional alpha-4 integrin protein, comprising the steps of:

(a) crossing two heterozygous alpha-4 integrin knockout mice comprising a first and second allele capable of expressing functional alpha-4 integrin protein, wherein the knockout mice each comprise a defect in either the first allele or second allele, such that either the first or second allele in each knockout mouse is unable to express functional alpha-4 integrin protein;

(b) harvesting the embryos resulting from the cross of step (a)

(c) inserting a transgene comprising a portion of an isolated cDNA molecule that encodes for alpha-4 integrin protein operatively associated with a promoter, into each embryo harvested in step (b), to form a transfected embryo having the transgene;

(d) inserting the transfected embryo into a pseudopregnant female mouse so that the pseudopregnant female mouse gives birth to a heterozygous alpha-4 knockout mouse whose genome comprises the transgene; and

(e) crossing two alpha-4 heterozygous knockout mice produced in step (d) to produce a homozygous alpha-4 knockout mouse whose genome comprises two copies of the transgene.

Numerous methods are readily available to the skilled artisan for placing a defect that disrupts the expression of an allele that encodes for alpha-4 integrin protein. For example, one such method is to employ knock-out technology to delete the alleles from the genome. Alternatively, recombinant techniques can be used to introduce mutations, such as nonsense and amber mutations, or mutations that lead to expression of non-functional alpha-4 integrin protein. In another embodiment, the alleles that encode for alpha-4 integrin protein can be tested for disruption by examining their phenotypic effects when expressed in antisense orientation in wild-type animals. In this approach, expression of the wild-type allele is suppressed, which leads to a mutant phenotype. RNA.RNA duplex formation (antisense-sense) prevents normal handling of mRNA, resulting in partial or complete elimination of wild-type gene effect. This technique has been used to inhibit TK synthesis in tissue culture and to produce phenotypes of the Kruppel mutation in Drosophila, and the Shiverer mutation in mice [Izant et al., Cell, 36:1007-1015 (1984); Green et al., Annu. Rev. Biochem., 55:569-597 (1986); Katsuki et al., Science, 241:593-595 (1988)]. An important advantage of this approach is that only a small portion of the gene need be expressed for effective inhibition of expression of the entire cognate mRNA. The antisense transgene will be placed under control of its own promoter or another promoter expressed in the correct cell type, and placed upstream of the SV40 polyA site. This transgene will be used to make transgenic mice, or by using gene knockout technology.

A particular heterozygous alpha-4 integrin knockout mouse having applications in methods of the present invention is available from Jackson Laboratories (Bar Harbor, Me.), and has been assigned Jackson Laboratories stock number 002463.

Other steps of a method of the present invention include harvesting the embryos that result from a cross of alpha-4 integrin heterozygous knockout mice, transfecting the embryos with a transgene comprising a portion of a cDNA molecule that encodes alpha-4 integrin operatively associated with a promoter, inserting the transfected embryo into a pseudopregnant mouse, crossing two heterozygous offspring, and then selecting offspring of this second cross (F₂ generation) that are homozygous alpha-4 integrin knockout, and have two copies of the transgene in their genome. Methods of performing these steps are well within the skill of one of ordinary skill in the art. For example, a transgene comprising a portion of a cDNA molecule that encodes for alpha-4 integrin operatively associated with a promoter can be inserted into an appropriate expression vector, i.e., a vector which contains the necessary elements for the transcription and translation of the inserted protein-coding sequence. The expression vector, in turn, is then used to transfect a harvested alpha-4 integrin heterozygous embryo. Optionally, the necessary transcriptional and translational signals can be provided on a recombinant expression vector, or they may be supplied by the native gene, i.e., a gene encoding alpha-4 integrin protein, and/or its flanking regions. Moreover, optionally, the expression vector can contain a replication origin.

Expression of the transgene once within the genome of the mouse may be controlled by any promoter/enhancer element known in the art, but these regulatory elements must be functional in the murine cell. Promoters which may be used to control expression of the transgene include, but are not limited to, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al, 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1:161 -171), beta-globin gene control region which is active in myeloid cells (Mogram et al., 1985, Nature 315:338-340; Kollias et al., 1986, Cell 46:89-94), myelin basic protein gene control region which is active in oligodendrocyte cells in the brain (Readhead et al., 1987, Cell 48:703-712), myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378). In a particular embodiment, the promoter utilized in the transgene is the tet-promoter. The tet-promoter consists of a virtually silent, minimal hCMV (human cytomegalovirus) promoter and several tetO (tet-Operator) sequences, named TRE (tet-responsive element). (Gossen and Bujard, 1992). Even though the minimal hCMV promoter is supposed to be silent, it's commonly found that transcriptional regulation is not tight, leading to low level “leakiness” of the following gene. Expression vectors containing the transgene can be identified by four general approaches: (a) PCR amplification of the desired plasmid DNA or specific mRNA, (b) nucleic acid hybridization, (c) presence or absence of selection marker gene functions, and (d) expression of inserted sequences. In the first approach, the nucleic acids can be amplified by PCR to provide for detection of the amplified product. In the second approach, the presence of a foreign gene inserted into an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the transgene is inserted within the “selection marker” gene sequence of the vector, recombinants containing the transgene can be identified by the absence of selector marker gene function. In the fourth approach, recombinant expression vectors can be identified by assaying for the activity, biochemical, or immunological characteristics of the truncated alpha-4 integrin protein encoded by the transgene. Antibodies to the truncated protein can readily be made using routine laboratory techniques. Particular methods known to produce antibodies include, but are not limited to the hybridoma technique originally developed by Kohler and Milstein [Nature 256:495-497 (1975)], as well as the trioma technique, the human B-cell hybridoma technique [Kozbor et al., Immunology Today 4:72 1983); Cote et al., Proc. Natl. Acad. Sci. USA. 80:2026-2030 (1983)], and the EBV-hybridoma technique to produce human monoclonal antibodies [Cole et al., in Monoclonal Antibodies and Cancer Therapy, Alan R. Liss, Inc., pp. 77-96 (1985)].

Mammalian expression vectors having applications herein include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; see Kaufmnan, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfI, XhoI, NotI, NheI, HindIII, NheI, PvuII, and KpnI cloning site, constitutive RSV-LTR promoter, hygromycin selectable marker; Invitrogen), pCEP4 (BamH1, SfI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamH1 cloning site, inducible metallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSC11 (SmaI cloning site, TK- and β-gal selection), pMJ601 (SalI, SmaI, AflI, NarI, BspMII, BamHI, ApaI, NheI, SacII, KpnI, and HindIII cloning site; TK- and β-gal selection), and pTKgptF1S (EcoRI, PstI, SalI, AccI, HindII, SbaI, BamHI, and Hpa cloning site, TK or XPRT selection).

Expression vectors are introduced into the embryo by methods readily known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

Likewise, steps involving the insertion of the transfected embryo into a pseudopregnant mouse are also readily understood and available to a skilled artisan. Particular examples of such techniques are clearly set forth in U.S. Pat. No. 5,175,385 to Wagner et al., and U.S. Pat. No. 5,929,043 to Dayn, both of which are hereby incorporated by reference in their entireties.

A Transgenic Mouse that is Unable to Express Functional Alpha-4 Integrin Protein

Furthermore, as explained above, the present invention extends to a transgenic mouse that is unable able to express functional alpha-4 integrin protein. In particular, a transgenic mouse of the present invention would have a genome in which first and second alleles capable of expressing functional alpha-4 integrin protein have been replaced with two copies of a transgene comprising a portion of an isolated cDNA molecule that encodes for alpha-4 integrin protein, operatively associated with a promoter. As a result, a transgenic mouse of the present invention would be unable to express functional alpha-4 integrin protein.

Naturally, the present invention extends to a method of producing a transgenic mouse that is unable to express functional alpha-4 integrin protein, comprising the steps of:

(a) crossing a first transgenic mouse having a genome in which an allele that is capable of expressing functional alpha-4 integrin protein is replaced with a transgene comprising a portion of an isolated cDNA molecule that encodes for alpha-4 integrin protein operatively associated with a promoter, with a second transgenic mouse having a genome in which an allele that is capable of expressing functional alpha-4 integrin protein is replaced with the transgene;

(b) selecting offspring from the cross that have a genome in which both alleles capable of expressing functional alpha-4 integrin protein have been replaced with two copies of the transgene.

These selected offspring are transgenic mice that are unable to express functional alpha-4 integrin protein, and thus are transgenic mice of the present invention.

Numerous methods are available to the skilled artisan to replace an allele that is capable of expressing alpha-4 integrin protein within the first and second transgenic mice with a transgene described above. One such method is gene targeting. “Gene targeting” is a type of homologous recombination that occurs when a fragment of genomic DNA is introduced into a mammalian cell, and that fragment locates and recombines with endogenous homologous sequences. It has been used in various systems, from yeast to mice, to make specific mutations in the genome. Gene targeting is not only useful for studying function of proteins in vivo, but is also useful for creating animal models for human diseases, and gene therapy The technique involves the homologous recombination between DNA introduced into a cell and the endogenous chromosomal DNA of the cell. As a result, this technique permits one to “swap” the endogenous DNA for exogenous DNA, such as, for example a transgene comprising SEQ ID NO: 1 operatively associated with a promoter. A particular method of gene targeting that can readily adapted using routine laboratory techniques for application in the present invention is described within U.S. Pat. No. 6,143,566, which is hereby incorporated by reference in its entirety. Techniques for transfecting embryos with an expression vector comprising the transgene, and inserting the transfected embryo into a pseudopregnant female to F₁ mice are described above. Naturally, these techniques readily have applications in making a transgenic mouse of the invention.

Methods for Assaying Compounds or Agents for the Ability to Modulate Alpha-4 Integrin Protein Activity

As explained above, it has been discovered that an organism's inability to express functional alpha-4 integrin protein results in surprising and unexpected phenotypical changes in the organism. Such organisms, along with information obtained on their phenotypes, have immediate applications in methods for assaying compounds or agents for the ability to modulate, and particularly antagonize alpha-4 integrin protein activity. In particular, it has been discovered that, surprisingly and unexpectedly, a lack of functional alpha-4 integrin modulates the levels of variety of genetic markers in a mammal. Examples of such markers include, but certainly are not limited to:

Mus musculus anti-von Willebrand factor antibody NMC-4 kappa chain mRNA;

Mouse gene for immunoglobulin alpha heavy chain, switch region and con;

(H-2 class I histocompatibility antigen, d-k alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete cds;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

mt23g11.r1 Soares mouse 3NbMS Mus musculus cDNA clone 621956 5′ TIGR cds;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: poliovirus receptor homolog precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

Mus musculus Bop1 mRNA, complete cds;

C75959 Mouse 3.5-dpc blastocyst cDNA Mus musculus cDNA clone J0001C05;

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA CLASS II histocompatibility antigen, DO B;

NRNT(3e-39): Human phosphatidylinositol (4,5)bisphosphate 5-phosphatase;

Mus musculus (clone U2) T-cell specific protein mRNA, complete cds;

Mus musculus mRNA for peroxisomal integral membrane protein PMP34;

M. musculus mRNA for macrophage mannose receptor; and

the concentration of progenitor stem cells in blood.

More specifically, it has been discovered that in the phenotype of a mouse of the present invention, the absence of functional alpha-4 integrin protein results in an increase in the measured level a variety of genetic markers, including, but not limited to:

Mus musculus anti-von Willebrand factor antibody NMC-4 kappa chain mRNA;

Mouse gene for immunoglobulin alpha heavy chain, switch region and con;

(H-2 CLASS I histocompatibility antigen, D-K alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete c;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

NRNT(0.0): Mus musculus mRNA for IIGP protein;

Mouse DNA for Ig gamma-chain, secrete-type and membrane-bound, partial;

NRNT(2e-6 1): Mus musculus DNA for PSMB5, complete cds;

Homologous to sp P32507: Poliovirus Receptor Homolog Precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

mt23g11.r1 Soares mouse 3NbMS Mus musculus cDNA clone 621956 5′ TIGR c;

Mus musculus Bop1 mRNA, complete cds;

C75959 Mouse 3.5-dpc blastocyst cDNA Mus musculus cDNA clone J0001C05; and

the concentration of progenitor stem cells in blood, to name only a few.

It has also been discovered that the measured level of some genetic markers in a mouse that is unable to express functional alpha-4 integrin protein decreases. Examples of genetic markers whose levels decrease include:

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA Class II histocompatibility antigen, DO B;

NRNT(3e-39): Human phosphatidylinositol (4,5)bisphosphate 5-phosphatase;

Mus musculus (clone U2) T-cell specific protein mRNA, complete cds; and

M. musculus mRNA for macrophage mannose receptor mRNA, to name only a few.

Using this information gleamed from a mouse of the present invention, methods for assaying compounds or agents for the ability to modulate, and particularly antagonize the activity of alpha-4 integrin antagonist activity have been discovered. Compounds or agents that antagonize alpha-4 integrin protein activity may have applications as therapeutic agents to treat a large variety of diseases or disorders including inflammation, asthma, arthritis, MS and others. Accordingly, the present invention extends to a method for assaying a compound or agent for the ability to modulate, and particularly antagonize the activity of alpha-4 integrin protein, comprising the steps of (a) administering the compound or agent to a wildtype mouse, (b) measuring the level of a genetic marker in the wildtype mouse, and (c) comparing the measurement of step (b) with the level of the genetic marker measured in a control wild type mouse. Modulation of the level of the genetic marker measured in the wild type mouse relative to the level of the genetic marker measured in the control wild type mouse indicates the compound or agent may possess alpha-4 integrin protein antagonist activity. Examples of genetic markers whose modulation is directly related to decreased alpha-4 integrin protein activity, as well as genetic markers whose levels are inversely related to decreased alpha-4 integrin protein activity are described above. Hence, a compound or agent that modulates the level of these genetic markers as described above has the ability to be an alpha-4 integrin protein antagonist. Thus, if the level of any of the following genetic markers increases after administration to the compound or agent, then the compound or agent may have alpha-4 integrin antagonist activity:

Mus musculus anti-von Willebrand factor antibody NMC-4 kappa chain mRNA;

Mouse gene for immunoglobulin alpha heavy chain, switch region and con;

(H-2 CLASS I histocompatibility antigen, D-K alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete c;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: Poliovirus Receptor Homolog Precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

mt23g11.r1 Soares mouse 3NbMS Mus musculus cDNA clone 621956 5′ TIGR c;

Mus musculus Bop1 mRNA, complete cds;

C75959 Mouse 3.5-dpc blastocyst cDNA Mus musculus cDNA clone J0001C05; and

the concentration of progenitor stem cells in blood,

Alternatively, if the level of any of the following genetic markers decreases after administration of the compound or agent, the compound or agent may possess alpha-4 integrin antagonist activity:

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA Class II histocompatibility antigen, DO B;

NRNT(3e-39): Human phosphatidylinositol (4,5)bisphosphate 5-phosphatase;

Mus musculus (clone U2) T-cell specific protein mRNA, complete cds; or

M. musculus mRNA for macrophage mannose receptor.

Moreover, rather than comparing a blood sample from an animal administered a compound or agent with a blood sample taken from a control mammal, a similar method involves comparing a blood sample taken from the mammal prior to administration of the compound or agent, and a blood sample taken from the same mammal after administration of the compound or agent.

Furthermore, the present invention extends to the use of mice that are not able to express functional alpha-4 integrin protein to assay compounds or agents that can ameliorate side effects associated with alpha-4 integrin antagonists or VLA-4 receptor antagonists. In particular, mice of the present invention are unable to express functional alpha-4 integrin protein. Thus, inherently, their modulated phenotypes result from a lack of functional alpha-4 integrin in the mammal. Hence, if a compound or agent administered to a mouse of the present invention modulates the level of genetic markers measured in the mouse in a direction towards the measured levels of the genetic markers measured in a wild type control mouse, then the compound or agent may have applications in treating side effects associated with alpha-4 integrin protein or VLA-4 receptor antagonists.

Accordingly, the present invention extends to a method for assaying a compound or agent for activity in ameliorating deleterious side effects associated with an alpha-4 integrin protein antagonist or a VLA-4 receptor antagonist, comprising the steps of:

(a) administering the compound or agent to a mouse unable to express functional alpha-4 integrin protein;

(b) measuring the level of a genetic marker in the mouse; and

(c) comparing the level of the genetic marker measured in the mouse to the level of the genetic marker measured in a control mouse of the present invention that is not able to express functional alpha-4 integrin protein,

wherein a modulation of the level of the genetic marker measured in the mouse to which the compound or agent was administered relative to the level of the genetic marker measured in the control mouse that is unable to express functional alpha-4 integrin protein indicates the compound or agent may have activity in ameliorating deleterious side effects associated with an alpha-4 integrin protein antagonist.

A compound or agent having applications in ameliorating deleterious side effects associated with an alpha-4 integrin antagonist or a VLA-4 receptor antagonist would modulate the measured level of genetic markers in directions opposite to their modulation as a result of administration of an alpha-4 integrin antagonist. Thus, for a particular genetic marker, if administration of an alpha-4 integrin antagonist decreases the measured level of the genetic marker, than a compound or agent for treating side effects associated with an alpha-4 integrin antagonist increases the measured level of the genetic marker, and vice versa. Examples of genetic markers having applications in such a method of the present invention are described above.

Numerous methods presently available can be used to administer a compound or agent in a method of the present invention. In particular, the compound or agent can be administered parenterally, e.g., via intravenous injection, and also including, but is not limited to, intra-arteriole, intramuscular, intradermal, subcutaneous, intraperitoneal, intraventricular, and intracranial administration. Still other methods of administering the compound or agent having applications herein are transmucosally, e.g., orally, nasally, or rectally, or transdermally.

Methods of drawing blood samples and measuring genetic markers are described infra.

Methods for Determining Whether a Compound or Agent has the Ability to Modulate the Signaling Activity of VLA-4 Receptor

As explained above, the level of genetic markers in a mouse of the present invention that is unable to express functional alpha-4 integrin have been discovered to be modulated relative to the level of these genetic markers found in a wildtype mouse. As explained above, some the genetic markers are modulated directly relative to the activity of alpha-4 integrin protein, and some of the genetic markers are modulated inversely relative to the activity of alpha-4 integrin protein. Moreover, also for reasons discussed above, the level of the signaling activity of VLA-4 receptor is directly related to the level alpha-4 integrin protein present. Consequently, a compound or agent administered to an organism that modulates the level a genetic marker for alpha-4 integrin protein in the organism relative to the level of the genetic marker in a control organism also has the ability to modulate the signaling activity of VLA-4 receptor. Thus, the genetic markers set forth above, and their modulation with respect to alpha-4 integrin protein are also genetic markers for the signaling activity of VLA-4 receptor, and are modulated in the same manner. Hence, the present invention extends to a method for determining whether a compound or agent modulates signaling activity of a VLA-4 receptor, comprising the steps of:

(a) administering the compound or agent to an organism;

(b) measuring the expression level of a genetic marker for VLA-4 receptor signaling in a bodily sample removed from the organism; and

(c) comparing the expression level of the genetic marker of step (b) with the expression level of the genetic marker measured in a control bodily sample.

A difference between the measured expression level of the genetic marker in the bodily sample and the control bodily sample indicates that the compound or agent modulates the signaling of the VLA-4 receptor. In particular, examples of genetic markers whose expression level increases, i.e., is inversely related to the signaling activity of VLA-4 receptor include, but certainly are not limited to:

Mus musculus anti-von Willebrand factor antibody NMC-4 kappa chain mRNA;

Mouse gene for immunoglobulin alpha heavy chain, switch region and con;

(H-2 CLASS I histocompatibility antigen, D-K alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete c;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: Poliovirus Receptor Homolog Precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE;

M.musculus mRNA for D2A dopamine receptor;

mo54b05.r1 Life Tech mouse embryo 10 5 dpc 10665016 Mus musculus cDNA cds;

mt23g11.r1 Soares mouse 3NbMS Mus musculus cDNA clone 621956 5′ TIGR c;

Mus musculus Bop1 mRNA, complete cds;

C75959 Mouse 3.5-dpc blastocyst cDNA Mus musculus cDNA clone J0001C05; and

the concentration of progenitor stem cells in blood.

Similarly, examples of genetic markers whose expression level decreases, i.e., is directly related to the signaling activity of VLA-4 receptor include:

vm06f11.r1 Knowles Solter mouse blastocyst B1 Mus musculus cDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA Class II histocompatibility antigen, DO B;

NRNT(3e-39): Human phosphatidylinositol (4,5)bisphosphate 5-phosphatase;

Mus musculus (clone U2) T-cell specific protein mRNA, complete cds; and

M. musculus mRNA for macrophage mannose receptor.

Naturally, a bodily sample includes, but certainly is not limited to a bodily fluid, e.g., blood, urine, saliva, mucus, semen, lymph, etc, or a solid sample such as tissue, bone, hair, etc. Moreover, the control bodily sample can be a bodily sample taken from the organism prior to the administration of the compound or agent, or alternatively, a bodily sample taken from a second organism substantially similar to the first organism (same or similar specie, age, weight, sex, etc.), to which the compound or agent is not administered. Naturally, an organism can be a mammal, including but not limited to ovine, bovine, equine, canine, feline, murine, or human, to name only a few.

A particular example of a genetic marker that is a “surrogate” marker for signaling activity of VLA-4 receptor, and whose expression level is directly related to the signaling activity of VLA-4 receptor is M. musculus mRNA for macrophage mannose receptor, which has been assigned GenBank Accession number: Z11974, and is set forth in SEQ ID NO: 13.

Another particular example of a genetic marker that is a “surrogate” marker for signaling activity of VLA-4 receptor, and whose expression level is directly related to the signaling activity of VLA-4 receptor is Mus musculus mRNA for JAB, complete cds, or SOCS-1 protein, whose nucleotide sequence has been assigned GenBank accession number AB000677, and is set forth in SEQ ID NO: 17.

Other particular examples of genetic markers that are “surrogate” markers for signaling activity of VLA-4 receptor, and whose expression levels are directly related to the signaling activity of VLA-4 receptor are EST AA571535 (vm06f11.r1 Knowles Solter mouse blastocyst B1 Mus musculus cDNA clone), having a nucleotide sequence of SEQ ID NO: 21, and EST AA154371 (Homologous to sp P13765: HLA CLASS II histocompatibility antigen, DO B), having a nucleotide sequence of SEQ ID NO: 23.

Similarly, the present invention extends to a method for determining the ability of a compound or agent to antagonize the signaling activity of a VLA-4 receptor, comprising the steps of:

(a) removing a first bodily sample from an organism;

(b) measuring the level of a genetic marker in the first bodily sample, wherein the genetic marker is selected from the group consisting of:

vm06f11.r1 Knowles Solter mouse blastocyst B1 Mus musculuscDNA clone;

Mus musculus Major Histocompatibility Locus class II region;

Mus musculus capping protein beta-subunit isoform 1 mRNA, complete cds;

Mus musculus mRNA for peroxisomal integral membrane protein PMP34;

Mus musculus mRNA for JAB, complete cds;

Mouse interferon regulatory factor 1 mRNA, complete cds;

Mus musculus GTPase IGTP mRNA, complete cds;

Mouse spi2 proteinase inhibitor (spi2/eb1) mRNA, 3 end;

Homologous to sp Q01514: Interferon-Induced Guanylate-Binding Protein;

Homologous to sp P13765: HLA Class II histocompatibility antigen, DO B;

NRNT(3e-39): Human phosphatidylinositol (4,5)bisphosphate 5-phosphatase;

Mus musculus (clone U2) T-cell specific protein mRNA, complete cds; and

M. musculus mRNA for macrophage mannose receptor,

(c) administering the potential antagonist to the organism;

(d) removing a second bodily sample from the organism;

(e) measuring the level of the genetic marker in the second bodily sample; and

(f) comparing the measured levels of step (b) and step (e).

A decrease in the measured level of the genetic marker in step (e) relative to the measured level of the genetic marker in step (b) indicates that the compound or agent is an antagonist of the signaling activity of VLA-4. Naturally, as described above, the first and second bodily samples may comprise a bodily fluid, a bodily tissue, or a combination thereof. Particular genetic markers having applications here are M. musculus mRNA for macrophage mannose receptor, which has been assigned GenBank Accession number: Z11974, and is set forth in SEQ ID NO: 13; Mus musculus mRNA for JAB, complete cds, or SOCS-1 protein, whose nucleotide sequence has been assigned GenBank accession number AB000677, and is set forth in SEQ ID NO: 17, EST AA571535 (vm06f11.r1 Knowles Solter mouse blastocyst B1 Mus musculus cDNA clone), having a nucleotide sequence of SEQ ID NO: 21, and EST AA154371 (Homologous to sp P13765: HLA CLASS II histocompatibility antigen, DO B), having a nucleotide sequence of SEQ ID NO: 23.

In addition, the present invention extends to a method for determining the ability of a compound or agent to antagonize the signaling activity of a VLA-4 receptor, comprising the steps of:

(a) removing a first bodily sample from an organism;

(b) measuring the level of a genetic marker in the first bodily sample, wherein the genetic marker is selected from the group consisting of:

Mus musculus anti-von Willebrand factor antibody NMC-4 kappa chain mRNA;

Mouse gene for immunoglobulin alpha heavy chain, switch region and con;

(H-2 CLASS I histocompatibility antigen, D-K alpha chain precursor;

Mus musculus MHC class I Qa-1a antigen mRNA, complete cds;

Mus musculus ribosomal protein L41 mRNA, complete cds;

Mouse MHC class I D-region cell surface antigen (D2d) gene, complete c;

Mus musculus mRNA for erythroid differentiation regulator, partial;

NRNT(1e-92):, complete sequence [Mus musculus];

vc50e11.r1 Knowles Solter mouse 2 cell Mus musculus cDNA clone 778028;

NRNT(0.0): Mus musculus mRNA for IIGP protein;

Mouse DNA for Ig gamma-chain, secrete-type and membrane-bound, partial;

NRNT(2e-61): Mus musculus DNA for PSMB5, complete cds;

Homologous to sp P32507: Poliovirus Receptor Homolog Precursor;

Mouse Ig rearranged H-chain mRNA constant region;

M.musculus mRNA RHAMM;

R74638 MDB0793 Mouse brain, Stratagene Mus musculus cDNA 3′end;

Mus musculus pale ear (ep mutant allele) mRNA, partial cds;

mj35h09.r1 Soares mouse embryo NbME13.5 14.5 Mus musculus cDNA clone 4;

MUSGS00761 Mouse 3′-directed cDNA; MUSGS00761; clone mb1494. TIGR clus;

Homologous to sp P41725: brain enriched hyaluronan binding protein PRE; M.musculus mRNA for D2A dopamine receptor;

mo54b05.r1 Life Tech mouse embryo 10 5 dpc 10665016 Mus musculus cDNA cds;

mt23g11.r1 Soares mouse 3NbMS Mus musculus cDNA clone 621956 5′ TIGR c;

Mus musculus Bop1 mRNA, complete cds;

C75959 Mouse 3.5-dpc blastocyst cDNA Mus musculus cDNA clone J0001C05; and

the concentration of progenitor stem cells in blood,

(c) administering the potential antagonist to the organism;

(d) removing a second bodily sample from the organism;

(e) measuring the level of the genetic marker in the second bodily sample; and

(f) comparing the measured levels of step (b) and step (e).

An increase in the measured level of the genetic marker in step (e) relative to the measured level of the genetic marker in step (b) indicates that the compound or agent is an antagonist of the signaling activity of VLA-4. Naturally, as described above, the first and second bodily samples may comprise a bodily fluid, a bodily tissue, or a combination thereof.

As explained above, numerous types of organisms have application in a method of the present invention. Particular examples include, but certainly are not limited mammals such as ovine, bovine, equine, canine, feline, murine, or human, to name only a few.

### Modulators of VLA-4 Receptor Signaling Activity

As explained above, a compound or agent that can be evaluated in a method of the present invention can be an antibody having a VLA-4 receptor as an immunogen, or a fragment of such a an antibody, or an antibody having alpha-4 integrin protein as an immunogen, or a fragment of such an antibody; a chemical compound; a nucleic acid molecule such as an antisense molecule that hybridizes to RNA encoding VLA-4 receptor or an alpha-4 integrin protein, or a ribozyme engineered to cleave RNA that encodes a VLA-4 receptor of an alpha-4 integrin protein; a carbohydrate; a hormone, or a lectin. Particular examples of such compounds or agents are described below.

a. Antibodies

One example of a compound or agent that modulates VLA-4 signaling activity is an antibody having either alpha-4 integrin or VLA-4 receptor as an immunogen. Such antibodies include but are not limited to polyclonal, monoclonal, chimeric, single chain, Fab fragments, and an Fab expression library. Furthermore, the anti-VLA-4 and anti-alpha-4 integrin antibodies may be cross reactive, e.g., they may recognize VLA-4 and alpha-4 integrin protein, respectively, from different species. Polyclonal antibodies have greater likelihood of cross reactivity. Alternatively, an antibody of the invention may be specific for a single form of VLA-4 or alpha-4 integrin protein, such as murine.

Various procedures known in the art may be used for the production of polyclonal antibodies to VLA-4 or alpha-4 integrin protein. For the production of antibody, various host animals can be immunized by injection with the VLA-4 or alpha-4 integrin protein, including but not limited to rabbits, mice, rats, sheep, goats, etc. In one embodiment, a VLA-4 receptor or alpha-4 integrin protein, or a fragment thereof, can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward a VLA-4 receptor or an alpha-4 integrin protein, or a fragment thereof, any technique that provides for the production of antibody molecules by continuous cell lines in culture may be used. These include, but are not limited to the hybridoma technique originally developed by Kohler and Milstein [Nature 256:495-497 (1975)], as well as the trioma technique, the human B-cell hybridoma technique [Kozbor et al., Immunology Today 4:72 1983); Cote et al., Proc. Natl. Acad. Sci. U.S.A. 80:2026-2030 (1983)], and the EBV-hybridoma technique to produce human monoclonal antibodies [Cole et al., in Monoclonal Antibodies and Cancer Therapy, Alan R. Liss, Inc., pp. 77-96 (1985)]. Optionally, monoclonal antibodies can be produced in germ-free animals [PCT/US90/02545]. Techniques developed for the production of “chimeric antibodies” [Morrison et al., J. Bacteriol. 159:870 (1984); Neuberger et al., Nature 312:604-608 (1984); Takeda et al., Nature 314:452-454 (1985)] by splicing the genes from a mouse antibody molecule specific for a VLA-4 receptor or alpha-4 integrin protein together with genes from a human antibody molecule of appropriate biological activity can be used; such antibodies are within the scope of this invention. Such human or humanized chimeric antibodies are preferred for use as VLA-4 receptor signaling antagonists, since the human or humanized antibodies are much less likely than xenogenic antibodies to induce an immune response, in particular an allergic response, themselves.

According to the invention, techniques described for the production of single chain antibodies [U.S. Pat. Nos. 5,476,786 and 5,132,405 to Huston; U.S. Pat. No. 4,946,778] can be adapted to produce a VLA-4 receptor or alpha-4 integrin protein specific single chain antibodies. An additional embodiment of the invention utilizes the techniques described for the construction of Fab expression libraries [Huse et al., Science 246:1275-1281 (1989)] to allow rapid and easy identification of monoclonal Fab fragments with the desired specificity for a VLA-4 receptor or alpha-4 integrin protein, or their derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a VLA-4 receptor or alpha-4 integrin protein, one may assay generated hybridomas for a product which binds to a VLA-4 receptor or alpha-4 integrin protein fragment containing such epitope. For selection of an antibody specific to a VLA-4 receptor or alpha-4 integrin protein from a particular species of animal, one can select on the basis of positive binding with VLA-4 or alpha-4 integrin protein expressed by or isolated from cells of that species of animal.

The foregoing antibodies can also be used in methods known in the art relating to the localization and activity of a VLA-4 receptor or alpha-4 integrin protein, e.g., for Western blotting, imaging a VLA-4 receptor or alpha-4 integrin protein in situ, measuring levels thereof in appropriate physiological samples, etc. using any of the detection techniques mentioned above or known in the art.

Naturally, antibodies that agonize or antagonize the activity of VLA-4 or alpha-4 integrin protein can be generated. Such antibodies can be tested using the assays described.

b. Antisense and Ribozymes

The present invention also extends to the preparation of antisense nucleotides and ribozymes that modulate the signaling activity of VLA-4 receptor or the activity of alpha-4 integrin protein, by modulating the expression of genes that encode these proteins. Modulating such expression, particularly reducing or interfering with it, results in such compounds or agents that exhibit VLA-4 receptor signaling antagonist activity. This approach utilizes antisense nucleic acids and ribozymes to block translation of a specific mRNA, either by masking that mRNA with an antisense nucleic acid or cleaving it with a ribozyme.

Antisense nucleic acids are DNA or RNA molecules that are complementary to at least a portion of a specific mRNA molecule [see Marcus-Sekura, Anal. Biochem. 172:298 (1988)]. In the cell, they hybridize to that mRNA, forming a double stranded molecule. The cell does not translate an mRNA in this double-stranded form. Therefore, antisense nucleic acids interfere with the expression of mRNA into protein. Oligomers of about fifteen nucleotides and molecules that hybridize to the AUG initiation codon will be particularly efficient, since they are easy to synthesize and are likely to pose fewer problems than larger molecules when introducing them into organ cells. Antisense methods have been used to inhibit the expression of many genes in vitro [Marcus-Sekura, 1988, supra; Hambor et al., J. Exp. Med. 168:1237 (1988)]. Optionally, synthetic antisense nucleotides contain phosphoester bond analogs, such as phosphorothiolates, or thioesters, rather than natural phosphoester bonds. Such phosphoester bond analogs are more resistant to degradation, and thus increase the stability, and therefore the efficacy, of the antisense nucleic acids.

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognize specific nucleotide sequences in an RNA molecule and cleave it [Cech, J. Am. Med. Assoc. 260:3030 (1988)]. Because they are sequence-specific, only mRNAs with particular sequences are inactivated.

Investigators have identified two types of ribozymes, Tetrahymena-type and “hammerhead”-type. Tetrahymena-type ribozymes recognize four-base sequences, while “hammerhead”-type recognize eleven- to eighteen-base sequences. The longer the recognition sequence, the more likely it is to occur exclusively in the target mRNA species. Therefore, hammerhead-type ribozymes are preferable to Tetrahymena-type ribozymes for inactivating a specific mRNA species, and eighteen base recognition sequences are preferable to shorter recognition sequences.

The DNA sequences encoding VLA-4 and alpha-4 integrin protein, which can be readily obtained by one of ordinary skill in the art (e.g., murine and human sequences can readily be obtained in GenBank, for example, murine alpha-4 integrin has GenBank accession number NM010576, human alpha-4 integrin has GenBank accession number XM039011, murine VLA-4 receptor has GenBank accession numberU497283, and a plethera of sequences that encode human VLA-4 receptor can be obtained from GenBank) may thus be used to prepare antisense molecules against and ribozymes that cleave mRNAs for VLA-4 receptor or alpha-4 integrin protein, thus modulating the signaling activity of VLA-4 receptor protein.

c. Organic Compounds

Organic compounds have been developed which modulate VLA-4 receptor signaling activity. For example, such compounds are set forth in U.S. Pat. Nos. 6,352,977 and published PCT patent application WO99/23063, which are hereby incorporated by reference in their entireties. A particular example of such a compound is an (S)-3-((S)-2-(4,4-dimethyl-3-(4-(3-(2-methylphenyl)ureido)benzyl)-2,5-dioxoimidazolidin-1-yl)-2-(2-methylpropyl)acetylamino)-3-phenylpropionic acid of the formula:

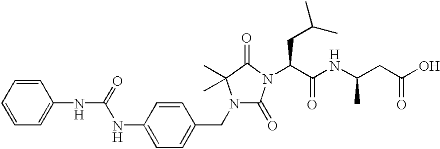

or a physiologically tolerable salt thereof, said compound being referred to herein as “HMR1031.”

Another example of such an organic compound has a formula of:

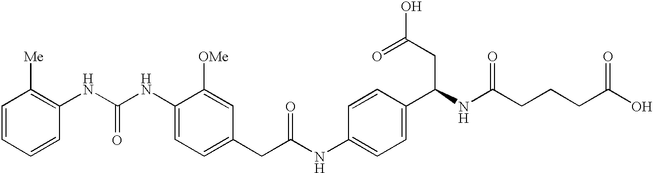

or a physiologically tolerable salt thereof, said compound being referred to herein as “IVL 984.”

Search of Libraries for Candidate Compounds or Agents that Modulate Signaling Activity of VLA-4 Receptor

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In a particular embodiment, high throughput screening methods involve providing a library containing a large number of potential therapeutic compounds (candidate compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### Combinatorial Chemical Libraries

Combinatorial chemical libraries are a preferred means to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 12331250).

Preparation of combinatorial chemical libraries is well known to those of ordinary skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random biooligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 69096913), vinylogous polypeptides (Hagihara et al. (1992) J. Amer. Chem. Soc. 114: 6568), nonpeptidal peptidomimetics with a Beta D Glucose scaffolding (Hirschmann et al., (1992) J. Amer. Chem. Soc. 114: 92179218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries, peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. Nos. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, HewlettPackard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

### High throughput Assays of Chemical Libraries

As explained above, the present invention extends to in vitro methods for determining whether a compound or agent modulates, and particularly antagonizes the signaling activity of VLA-4 receptor, comprising the steps of:

(d) contacting the compound or agent with a bodily sample from an organism;

(e) measuring the expression level of a genetic marker for VLA-4 receptor signaling in the bodily sample; and

(f) comparing the expression level of the genetic marker measured in step (b) with the expression level of the genetic marker measured in a control bodily sample.

Naturally, such a method is amenable to high throughput screening. High throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high thruput and rapid start up as well as a high degree of flexibility and customization. The manufacturers of such systems provide detailed protocols for the various high throughput. Thus, for example, Zymark Corp. provides technical bulletins describing screening systems for detecting the modulation of gene transcription, ligand binding, and the like.

The present invention may be better understood by reference to the following non-limiting Examples, which are provided as exemplary of the invention. The following Examples are presented in order to more fully illustrate the preferred embodiments of the invention. They should in no way be construed, however, as limiting the broad scope of the invention.

### EXAMPLE I

### Preparation of a Knockout Mouse that is Unable to Express Functional Alpha-4 Integrin Protein

Provided herein is a mouse that is unable to express functional alpha-4 integrin protein, along with a method for making such a mouse. A mouse unable to express functional alpha-4 integrin is a valuable tool for gaining further insight into leukocyte adhesion and migration into sites of inflammation and trafficking. Moreover, such a mouse of the present invention has valuable utility for screening of potential alpha-4 integrin antagonists for the treatment of a variety of diseases or disorders, including, but not limited to rheumatoid arthritis and asthma.

Materials And Methods

General Buffers and Solutions

| | |
|---|---|
| 10x TE buffer | 10 mM Tris-HCl (pH 8.0) |
| | 1 mM EDTA |
| 10x TAE buffer | 400 mM Tris-Acetate |
| | 10 mM EDTA |
| 20x SSC | 3 M NaCl |
| | 0.3 M Na-citrate |
| 5x TBE | 54 g Tris base |
| | 27.5 g Boric acid |
| | 20 ml 0.5 M EDTA, pH 8 |
| | H₂O ad 1 liter |
| LB medium | 10 g/liter casein hydrolysate (Bacto-tryptone) |
| | 5 g/liter yeast extract |
| | 10 g/liter NaCl |
| | pH adjusted to 7.0 with NaOH before autoclaving |
| Low salt LB medium | 10 g/liter casein hydrolysate (Bacto tryptone) |
| | 5 g/liter yeast extract |
| | 5 g/liter NaCl |
| | pH adjusted to 7.5 with NaOH before autoclaving |
| 10x PBS | 0.01 M KH₂PO₄ |
| | 0.1 M Na₂HPO₄ |
| | 1.37 M NaCl |
| | 0.027 M KCl |

Plasmids

| Name | Purpose | Supplier |
|---|---|---|
| pBSK- | Cloning of alpha-4 Integrin cDNA | Stratagene |
| pCR2.1 | TA-cloning of PCR products | Invitrogen |
| pCR TOPO | TA-cloning of PCR products | Invitrogen |
| pTet-Splice | Cloning of cDNA transgene | Gibco, Life |
| | | Technologies |
| | | Technologies |

Oligodeoxnribonucleotides

| | | |
|---|---|---|
| Oligo | Purpose | Sequence (5′ to 3′) |

| | | | |
|---|---|---|---|
| FV | PCR and PCR cloning | TCT TCT CTT TGG CCA ACC GT | (SEQ ID NO:2) |
| RM | PCR | GCA GGT CTG GTT TGG ATT CT | (SEQ ID NO:3) |
| ItgnII-F | PCR | CGC CTG CCA GCA CCG GAC A | (SEQ ID NO:4) |
| ItgnII-R | PCR | AGA GGC GGA GGC GCT GTG AC | (SEQ ID NO:5) |
| Neo-F2 | PCR | GCT GAC CGC TTC CTC GTG CTT TAC | (SEQ ID NO:6) |
| TetP-Therion | PCR | CAG ATC GCC TGG AGA CGC | (SEQ ID NO:7) |
| CDNA1B-R | PCR cloning | CAA CTT ATC ATC TTG AGG | (SEQ ID NO:8) |
| CDNA2- F | PCR cloning | TGG CTC CAA ATG TTA GTG | (SEQ ID NO:9) |
| CDNA2- R | PCR cloning | GGA GTG GAT CCT AGG AAA GGG | (SEQ ID NO:10) |
| | | GAT AAC ATT | |

Antibodies

| | |
|---|---|
| Immunohistochemistry | CD49d, BD Pharmingen |
| | clone 9C10 |

Methods

DNA Manipulation

(a) Ligation of DNA Fragments into Plasmids

To ligate an insert into a plasmid, both the plasmid containing the desired insert, as well as the recipient plasmid were digested with the appropriate restriction enzymes. If necessary, the fragments were filled in with Klenow and the recipient plasmid was dephosphorylated to prevent re-annealing. The insert fragment was separated from its parent plasmid by running the digest on an agarose gel for size separation and extraction of the desired band by phenol extraction. Typically the recipient plasmid was also extracted from an agarose gel after the dephosphorylation. For the ligation reaction, usually 100 ng of recipient vector was mixed with the appropriate amount of insert fraction to create molar ratios of 1:1, 1:3 and 1:5. For the insertion of adapters into a vector, molar ratios of 1:50 and 1:100 were used. To the reaction, 2 μl of 10×buffer and 1 μl of T4 ligase=10 units (Gibco, Life Technologies) were added into a total volume of 20 μl. The reactions were allowed to proceed at 16° C. overnight (12-16 hours). For the cloning of PCR products, the TA-cloning kit from Invitrogen was used. The kit is based on the fact that during a PCR reaction, the Taq polymerase adds a single deoxyadenosine to the 3′ ends of PCR products. The linearized vector provided by the kit has single deoxythymidine residues to allow the PCR product to be efficiently ligated with the vector.

(b) Transformation of Plasmid DNA into Competent E. coli Cells

In this work three different types of competent E. coli cells were used: DH5_{α} cells from Gibco, Life Technologies, XL-1 blue cells from Stratagene and INV_{α}F′ cells from Invitrogen. The transformation reactions were carried out according to the suppliers' protocols. The transformation reactions were plated on LP plates, containing the appropriate antibiotic at a concentration of 100 μg/ml. The plates were incubated at 37° C. overnight.

Ampicillin stocks: Ampicillin was stored as a 100 mg/ml stock solution in water at −20° C. and was added to the LB medium after autoclaving and cooling to at least 45° C. to yield a final concentration of 50 μg/ml.

LB-agar plates: To LB medium, 1.5% agar was added before autoclaving. After cooling to ˜45° C., the solution is poured into 10 cm dishes under sterile conditions. The desired antibiotic was added to the plates, prior to plating the transformed bacteria.

Low salt LB plates: Same procedure as for LB-agar plates but with low salt LB medium.

(c) Growth of Plasmid-Containing Bacterial Cultures

Single colonies that were grown on the antibiotic LB-plates were picked and used to inoculate 5 ml LB medium (or low salt LB medium) containing the appropriate antibiotic at a concentration of 50 μg/ml. For small-scale plasmid DNA preparation, this solution was incubated in a 37° C. incubator shaker for 12-16 hours at 225 rpm. For large scale plasmid DNA preparations, the inoculated 5 ml LB culture was grown for ˜5 hours and then transferred to 100 ml LB medium containing the appropriate antibiotic at a concentration of 50 μg/ml. This cell suspension was allowed to grow for an additional 12-16 hours.

(d) Small Scale Plasmid DNA Preparation (“Mini-prep”)

For a rapid isolation of plasmid DNA from single bacterial colonies, the “QIAspin Plasmid Kit” or the “QIAprep 8 Plasmid Kit” (Qiagen) were used. The later kit was used when more than 20 colonies had to be analyzed. In both cases, the protocol provided by the supplier was followed with a starting material of typically 1.5 ml overnight LB-culture. The principle of the kit is based on a modified alkaline lysis procedure and binding of the DNA to an anion exchange matrix. During the cell lysis, RNA is destroyed through the addition of RNase to the lysis buffer provided by the kit. All the wash and elution buffers are included in the kit and the final elution step of the DNA is carried out with water or TE. The purified plasmid DNA was analyzed by restriction digest or sequencing and stored at −20° C.

(e) Large Scale Plasmid DNA Preparation (“Maxi-prep”)

To purify a larger amount of plasmid DNA the “Qiagen Plasmid Maxi Kit” (Qiagen) was used. The principle of the kit is similar to the “mini-prep” kit, except that the DNA is eluted in a high salt buffer and thus the DNA has to be concentrated and desalted by isopropanol precipitation and washing of the DNA pellet with 70% ethanol. Typically, 100 ml of culture were used with one “Qiagen-tip 500” included in the kit.

(f) DNA Extraction from an Agarose Gel

The DNA was run on a 0.7% agarose gel containing ethidium bromide, the desired fragment was detected by UV-light, cut out, chopped and transferred into a 1.5 ml tube. To this, agarose/DNA fragment ˜750 μl of Phenol:Tris (no Chloroform) (Sigma) was added, vortexed and frozen down at −80° C. for at least 30 minutes. The tube was then centrifuged at high speed in a tabletop microfuge for 10 minutes. The supernatant was taken off and saved. More water or TE was added to the phenol-phase, and the tube was vortexed and frozen at −80° C. for another 30 minutes (or longer). The centrifugation step was repeated and the supernatants pooled. To this pool an equal volume of Phenol:Chlororoform (Sigma) was added, vortexed and spun down. The supernatant was ethanol precipitated and the final pellet resuspended in water or TE.

Sequencing

Sequencing of the pNEB 3.7(−) plasmid was done under the premises of TOPLAB GmbH (Martiensried, Germany) by the primer walking method. Both strands were sequenced.

Generation of Transgenic Mice

(a) Microinjecting and Breeding

Standard microinjection techniques are described in detail in “Manipulating the Mouse Embryo—A Laboratory Manual” (Hogan et al., 2nd ed. 1994, Cold Spring Harbor Laboratory Press, ISBN 0-87969-384-3) which is hereby incorporated by reference in its entirety. Such protocols have ready applications in methods for making a mouse of the present invention.

| Transgene(s) | Oocyte donor | Sperm donor |
|---|---|---|
| tetp-VLA | Alpha-4 Integrin heterozygous | Alpha-4 Integrin heterozy- |
| | knockout | gous knockout |

The mice heterozygous for the alpha-4 Integrin knockout were purchased from Jackson Laboratories, Bar Harbor, Me. (stock number 002463). These mice have a C57BL6/J background.

(b) Extraction of Genomic DNA from 3 Weeks Old Mouse Tails

About 1 cm of the tail tissue was removed from 3-4 week old mice. This tailpiece was placed into a 15 ml SST tube (Becton and Dickinson), and 750 μl of a tail digestion buffer was added. (Tail digestion buffer: 450 mls DI water; 5 mls 1M Tris, pH 7.5; 10 mls 5 M NaCl; 10 mls 0.5 M EDTA; plus 25 mls of 10% SDS after sterilfiltration). The tubes were sealed, placed in an incubator shaker, and the tails digested at 54° C. at 225 rpm. After 12-16 hours, 750 μl of phenol:chloroform:isoamylalcohol [1:1 (24:1)] (Sigma) was added, and the tubes were gently mixed for 15-30 seconds. To separate the phases, the tubes were centrifuged for 15 minutes at 4000 rpm at room temperature. The upper, aqueous phase was transferred to a 6 ml Falcon tube and 2 volumes of 96% ethanol were added. The genomic DNA precipitates were spooled out of the solution and transferred into a 96-well plate. The DNA was allowed to dry at room temperature for 1-2 hours, then it was resuspended in 200 μl TE and stored at −20° C. or processed immediately. For PCR reactions typically 1 μl of a 1:10 dilution was used as the template, for southern blotting 30 μl of undiluted DNA were used.

(c) PCR Testing of Genomic Tail DNA

Each DNA sample generated by the tail prep had to be analyzed for the endogenous alpha-4 Integrin knockout as well as for the presence of the transgenic DNA. In a particular embodiment of the present invention, the transgenic DNA comprises a DNA sequence of SEQ ID NO: 1. The DNA solution was diluted 1:10 in water, heated at 95° C. for 5 minutes and then placed on ice. 1 μl of the diluted DNA was used per PCR reaction. The reactions were carried out using AmpliTaq enzyme by Perkin Elmer (2.5 units/reaction), 0.2 mM dNTP and 1×buffer G from Invitrogen in a 50 μl volume. The PCR conditions as well as the primer concentrations were optimized for the specific target DNA. All PCR conditions contained an initial step of denaturing the DNA, carried out at 92-94° C. for 2-4 minutes, then typically 35-40 cycles of a three step procedure, consisting of 20-45 seconds of DNA melting at 92-94° C., 30-60 seconds of primer annealing at 55-64° C., depending on the melting temperature of the primer and Taq-mediated DNA synthesis at 72° C. for 30-60 seconds, depending on the length of the PCR product. Following those 35-40 cycles an extended step at 72° C. for 5-10 minutes was performed to ensure that all PCR products resulted in the same lengths. The PCR products were then analyzed by gel electrophoresis.

Integrin PCR to Determine the Background of the Endogenous Alpha-4 Integrin Gene

Primers: ItgnII-F, ItgnII-R, NeoF-2 in a final concentration of 25, 50 and 25 μM respectively. PCR conditions:

| 94° C. | 4 minutes | 1 cycle |
|---|---|---|
| 94° C. | 45 seconds | 35 cycles |
| 61° C. | 45 seconds | |
| 72° C. | 1 minutes with 10 seconds autoextension | |
| 72° C. | 10 minutes | 1 cycle |

Tet-Promoter PCR to Determine the Presence of the tetP-VLA Transgene

Primers: TetP-Therion and rM in a final concentration of 0.4 μM each PCR conditions:

| | | |
|---|---|---|
| 94° C. | 3 minutes | 1 cycle |
| 94° C. | 45 seconds | 35 cycles |
| 55° C. | 30 seconds | |
| 72° C. | 30 seconds | |
| 72° C. | 10 minutes | 1 cycle |

Collection of Tissue and Blood Samples from Transgenic Mice

For the tissue collection, mice were sacrificed by cervical dislocation. The desired tissue was removed under sterile conditions and placed into a pre-cooled 24-well tissue culture dish on dry ice. The samples were then stored at −80° C. until usage. For the collection of blood, the mice were anaesthetized with an i.p. injection of 2.5% Avertin, typically 0.4 to 0.6 ml per adult mouse. The blood samples were taken by orbital bleeding as follows: the head was secured between thumb and forefinger. A capillary tube was inserted at the medial edge of the eyeball and directed toward the back of the eye socket. The blood sinus was punctured by carefully rotating the capillary tube. The blood was collected in 0.5M EDTA tubes (Fisher Scientific) on ice to prevent the blood from coagulating.

Primary Tissue Culture

(a) Spleen Cell Isolation

Mice were sacrificed by cervical dislocation, the spleen aseptically removed and placed in a 50 ml centrifuge tube containing RPMI 1640 medium with 1% FCS. The spleens were then individually meshed in a 10 cm petri dish through a sterile wire screen using a sterile rubber policeman (0.23 mm pore size screen, Thomas Scientific). The screen was washed and the cell suspension collected and transferred into a 15 ml centrifuge tube. After centrifugation at 1200 rpm for 10 minutes at 4° C., the supernatant was decanted and the red blood cells were lysed with 1 ml/spleen ice-cold red blood cell lysis buffer for 1-3 minutes on ice. The supernatant was then carefully transferred to a fresh 15 ml centrifuge tube, leaving the fat pellet behind. The cell suspension was centrifuged for 10 minutes at 1200 rpm at 4° C. The supernatant was discarded and the cells resuspended in PBS and placed on ice. The number of live cells was determined using a hemacytometer (Hauser Scientific) and Trypan Blue (Gibco, Life Technologies) as the dye.

Red blood cell lysis buffer: 8.29 g NH₄Cl; 0.037 g EDTA; 1 g KHCO₃, Water add 1 liter, steril-filter.

(b) Leukocyte Tissue Culture

Leukocytes prepared by the spleen cell isolation protocol were cultured at 5% CO₂ at 37° C. in a starting concentration of 10⁷ cells/ml.

Culture Medium:

| | | |
|---|---|---|
| RPMI 1640 with glutamine | | |
| without phenol red | 434.5 ml | Gibco, Life Technologies |
| 2-mercaptoethanol | 0.5 ml | Gibco, Life Technologies |
| Kanamycin (100x) | 5 ml | Gibco, Life Technologies |
| Heat inactivated | | |
| Tet-System approved FBS | 50 ml | Clontech |
| Pen/Strep (100x) | 5 ml | Gibco, Life Technologies |
| L-glutamine (200 mM) | 5 ml | Gibco, Life Technologies |

(c) RNA Extraction from Leukocytes

To extract the RNA from leukocytes, the RNeasy Blood Mini kit” (Qiagen) was utilized. The erythrocyte lysis step with the kit reagents was skipped and the protocol was started at the lysis step of the leukocytes.

(b) Immunohistochemistry (IHC) Protocol: Preparation of Frozen Tissue and Staining:

The spleens were placed into a partly filled base mold with frozen tissue matrix. The base mold was then plunged into 2-methylbutane prechilled in a dewar of liquid nitrogen until the block almost solidified (about 30 seconds). The block was then placed on dry ice and stored frozen at −70° C. until sectioning.

For sectioning, the blocks were mounted on the cryostat chuck. 0.5 micron sections were cut and placed on a double frosted slide. The sections were fixed in cold (−20° C.) acetone for 2 minutes, dried completely and then stored at −70° C. until further use.

For the staining the common protocol as published by BD Pharmingen in their regular Research Products catalogue was utilized:

Slides were allowed to warm up to room temperature and were then rinsed 3× in PBS. A 0.03% H₂O₂ solution in PBS was applied for 10 minutes, and the slides were then rinsed again in PBS. A 5% serum solution was applied for 10-30 minutes, tapped off, and then the antibody solution was added and incubated for 1 hour in a humid chamber. The antibody used was CD49d, clone 9C10 (rat anti mouse) (BD Pharmingen). The slides were then rinsed 3× for 2 minutes in PBS. The slides were then incubated with biotin labeled anti-rat IgG antibody for 30 minutes at room temperature followed by a 3×rinse in PBS for 2 minutes each. The slides were drained and DAB solution (diamminobenzidine) was added for 5 minutes. Excess DAB was drained off and the slides were placed in a staining rack in a dish of water. The slides were rinsed in water 3× and then counterstained: The slides were dipped 2× in Hematoxylin, rinsed in water, dipped 2× in Bluing Reagent and rinsed in water. The slides were then submerged for 15 minutes each in the following solutions: 96% ethanol, 80% ethanol, 96% ethanol, xylanol. Permount (Fisher Scientific) was dripped onto the slide and covered with a glass coverslip.

RNA Manipulations

(a) RNA Extraction from Tissue Samples

The frozen samples were placed on dry ice to prevent thawing and thus degradation of the RNA. Each tissue sample was placed into 3.8 ml of the lysis buffer provided in the “RNeasy Midi kit” (Qiagen) and homogenized with a PT3100 Polytron for about 1 minute. The lysis is carried out under highly denaturing conditions in order to inactivate RNases. The protocol of the “RNeasy Midi kit” was then followed precisely. The principle of the kit is based on the ability of total RNA longer than 200 nucleotides to adsorb to the Silica membrane columns provided in the kit. The membrane with the adsorbed RNA is then washed several times to separate the RNA from contaminants and then eluted with water. Following the protocol, an ethanol precipitation of the RNA was conducted to ensure effective elimination of any residual ethanol in the samples. The ethanol precipitation was done at −20° C. for 12-16 hours, the RNA spun down and the pellet resuspended in RNase free water and stored at −20° C.

(b) RNA Extraction from Blood Samples

Freshly taken blood, stored on ice in EDTA tubes, was processed for RNA extraction according to the “RNeasy Blood Mini kit” (Qiagen) and following the instructions described therein. The obtained purified RNA was stored at −20° C. until further usage.

(d) Affymetrix Gene Chip Analysis

The RNA to be analyzed in this experiment was obtained from 5×C57 mice (the C57 line is described infra) and homozygous KO males of the present invention (line 59) each. All mice were sacrificed. The spleens of the mice were harvested, and put into 5 ml RPMI/1% FCS media on ice. Leukocytes were isolated as described above. For each individual, the total number of leukocytes was assessed. One half of the cells were used for an immediate RNA preparation, following the protocol of the RNeasy Blood Mini Kit (Qiagen). The other half of the cells were transferred into tissue culture with an addition of 1 μg/ml LPS (Sigma). The cells were harvested after 48 hours and the RNA was prepared. After each RNA preparation, an EtOH precipitation was performed in order to get a higher concentrated RNA sample. The RNA samples used in the probe synthesis had a minimal concentration of 0.5 μg/μl.

(i) Double Stranded cDNA Synthesis

For the generation of double stranded cDNA from total RNA, the “Superscript Choice System for cDNA Synthesis (Gibco, Life Technologies) was used.

(ii) First Strand cDNA Synthesis

10 μg of total RNA were mixed with 100 pmol T7-T(₂₄) primer (GGC CAG TGA ATT GTA ATA CGA CTC ACT ATA GGG AGG CGG-(T)₂₄) (SEQ ID NO: 11) and brought up to a final volume of 11.5 μl with RNase free water. For annealing of the primer to the template, the mix was incubated for 10 minutes at 70° C., quickly spun down and put on ice. A master mix containing the following components per sample was prepared on ice: 4 μl of 5×ist cDNA buffer, 2 μl 0.1 M DTT, 1 μl (10 mM) dNTP mix, 1.5 μl SSII RT enzyme. As soon as the master mix was prepared, the RNA samples were transferred to RT, the master mix was warmed up to ˜37° C. for about 2 minutes and 8.5 μl master mix was aliquoted into each tube. After mixing this reaction was incubated at 42° C. for 1 hour. The T7-T(24) primer annealed to the polyA tail of the total RNA, and was extended by the SSII RT enzyme using the nucleotides provided. The primer also incorporated a T7 promoter, which was used in subsequent steps.

(iii) Second Strand Synthesis

The first stand reactions were placed on ice after a quick spin and 60 μl of a master mix containing the following components were added: 4 μl of 2M KCl, 2 μl of Tris 1M pH 7.7 at RT, 0.4 μl of 1M MgCl₂, 2 μl of dNTP (10 mM) mix, 0.5 μl [2 U/μl] RNaseH, 2 μl [10 U/μl] E. Coli DNA polymerase I, 1 μl [10 U/μl ] E. Coli DNA ligase, water to a final volume of 60 μl (48.1 μl).

This mix was incubated at 16° C. for 2 hours. During this incubation, the second cDNA strand was generated as well as the remaining single stranded RNA destroyed. At the end of the first strand synthesis, a hairpin structure was formed that loops around the 5′ end of the RNA. This loop was then used as the start for the synthesis of the second strand. Therefore, no additional primer was added. After the 2 hour incubation, 2 μl (10 units) of T4 polymerase were added and incubated for 5 minutes at 16° C. in order to generate double stranded cDNA without overhangs.

To stop the reaction, 10 μl of 0.5 M EDTA was added, and the samples were stored at −20° C.

(iv) Clean up of Double Stranded cDNA

The double stranded cDNA was subjected to a Phenol:Chloroform: Isoamylalcohol (25:24:1, saturated with 10 mM Tris-HCl, pH 8.0, 1 mM EDTA) extraction, followed by an ethanol precipitation and immediate spin at RT for 5 minutes, 12,000 rpm. The pellet was washed 2×with 80% ice-cold EtOH and finally resuspended in 2 μl water.

(v) Synthesis of Biotin-Labeled RNA through In Vitro Transcription (IVT)

In this step, reagents from Ambion's T7 Megascript System were used. NTP labeling mix for 4 IVT reactions:

For 4 IVT reactions, the following components were combined on ice: 8 μl 10×ATP, 8 μl 10×GTP, 6 μL 10×CTP, 6 μl 10×UTP (all NTPs at 75 mM, Ambion), 15 μl Bio-11-CTP and 15 μl Bio-16-UTP (both at 10 mM, Enzo Diagnostics). Excess NTP labeling mix was stored at −20° C. until further usage.

(vii) In Vitro Transcription (IVT) Reaction:

For each reaction, the following reagents were combined at room temperature (RT): 14.5 μl NTP labeling mix, 2 μl 10×transcription buffer (Ambion), 2 μl 10×T7 enzyme mix (Ambion), 1.5 μl ds cDNA. The mix was incubated at 37° C. for 5 hours.

(viii) Cleaning up IVT Products:

During this step, the RNeasy Midi Kit (Qiagen) was utilized in order to remove unincorporated NTPs as described above. After the final elution of the cRNA, an EtOH precipitation with 0.5 volumes of 5 M NH₄Ac and 2.5 volumes absolute EtOH was performed to ensure that the final cRNA was free of any residual EtOH. This precipitation was done overnight at −20° C. To pellet the cRNA, the precipitation solution was spun down at 14,000 g for 30 minutes at 4° C., the pellet was washed 2×with ice-cold 80% EtOH and finally resuspended in 16 μl water. 1 μl was used to determine the concentration by UV measurement at 260 and 280 nm, 1 μl was used to determine the average length of the IVT products on a 1% agarose gel.

Target Hybridization

(a) Fragmentation of IVT Product

To fragment the cRNA for future hybridization, about 20 μg cRNA were mixed with 4 μl 5×fragmentation buffer and water to a final volume of 20 μl.

This mixture was incubated for 35 minutes at 95° C.

5×fragmentation buffer: 1 M Tris-acetate, pH 8.1 4 ml, MgOAc 0.64 g, KOAc 0.98 g, water to a final volume of 20 ml, filtered through a 0.2 μm filter unit and stored at 4° C.

(b) Preparing the Hybridization Target

To the 20 μl fragmented cRNA the following mixture was added: 150 μl 2×MES hybridization buffer, 3 μl Herring sperm DNA (10 mg/ml), 3 μl 100×control BioB, BioC, BioD and Cre cocktail, 3 μl acetylated BSA (50 mg/ml), 3 μl Control Oligonucleotide B2 (5 nM), 118 μl water.

BioB, BioC and BioD are bacterial genes of the biotin synthesis pathway. Cre is a phage gene from P1 bacteriophage. Those genes were transcribed into biotin labeled cRNA with a similar protocol as described above, fragmented and stored in aliquots to give a final concentration of 15 nM, 50 nM, 250 nM and 1 μM respectively. The 100×control cRNA cocktail was mixed as follows: 10 μl of each control aliquot, 10 μl Herring Sperm DNA (10 mg/ml), 12×MES: 83.3 μl, 5 M NaCl 185 μl, 1 μl Tween20 (10%), 680.7 μl water.

The B2 biotinylated oligonucleotide hybridized to the sides and corners of each Affymetrix chip to allow the scanner to align the grid after staining and scanning. Oligo B2: 5′ bio GTC AAG ATG CTA CCG TTC AG 3′ (SEQ ID NO: 12).

2×MES hybridization buffer: 8.3 ml of 12×MES stock, 17.7 ml of 5 M NaCl, 4 ml of 0.5 M EDTA, 0.1 ml of 10% Tween 20, 19.9 ml water.

12×MES stock: 70.4 g MES free acid monohydrate, 193.3 g MES Sodium Salt, 800 ml water, pH between 6.5 and 6.7, total volume 1000 ml, filtered through a 0.2 μm filter.

(c) Target Cleanup and Hybridization

The Affymetrix chips were equilibrated to RT immediately before use. The hybridization cocktail was heated to 99° C. for 5 minutes. In the meantime the chips were wetted for 10 to up to 60 minutes with 200 μl 1×MES at 45° C. with rotation (60 rpm). The heated samples were spun in a microcentrifuge for 5 minutes at full speed to remove any insoluble material from the hybridization mixture. The 1×MES buffer was removed from the chip and replaced with 200 μl of the clarified hybridization mixture. Hybridization was carried out at 45° C. in a rotisserie box, rotating at 60 rpm overnight (about 16 h). The remaining hybridization mixture was stored at −20° C.

(d) Washing and Standard Staining of the Probe Array

The washing and staining was done using a GeneChip Fluidics Station 400. Prior to the staining the machine has to be primed with the appropriate buffers A and B.

Buffer A (non-stringent): 6×SSPE, 0.01% Tween-20, 0.005% Antifoam, filtered through a 1.2 μm filter unit.

Buffer B (stringent): 0.5×SSPE, 0.01% Tween-20, filtered through a 0.2 μm filter unit.

After the hybridization, the hybridization mixture was removed from the chip and combined with the leftovers from the previous day. The mixture was then stored at −80° C. until other hybridization. The chips were filled with 200 μl buffer A.

For one probe array the following reagents were mixed together: 600 μl 2×stain buffer, 48 μl, BSA (50 mg/ml), 12 μl streptavidin phycoerythrin (SAPE) (1 mg/ml), 540 μl water.

2×stain buffer: 200 mM MES, 2 M Na⁺, 0.1%Tween20, 0.01% Antifoam.

The chip was stained with this solution and washed several times with buffer A and B and water according to the machines'set protocol EukGE-WS2 (GeneChip Software). After the SAPE staining, in order to intensify the signal, the chips were stained with the following antibody solution: 300 μl of 2×stain buffer, 24 μl of 50 mg/ml acetylated BSA, 6 μl of 10 mg/ml normal goat IgG, 3.6 μl of 0.5 mg/ml biotinylated antibody, 266.4 μl water per chip. After the stain, the chips were washed again and a third staining with the SAPE solution was performed.

(e) Probe Array Scan

The scanner was controlled by the GeneChip Software. Prior to loading the probe array into the scanner, the window of the chip had to be checked for any air-bubbles. In case air-bubbles were present, they had to be removed with filling more buffer A into the chip. Each chip was scanned twice and the primary data analysis was done on that machine before the data was sent off for Affymetrix analysis.

Affymetrix Analysis

An Affymetrix analysis was performed using an Affymetrix Data Mining Tool (Affymetrix, Santa Clara, Calif.). In the first step, one virtual chip that combined the A and the B murine 11K chips used in this experiment was created. One virtual chip per mouse per treatment group, resulting in 20 virtual chips total (5 KO mice of the invention, and 5 C57/BL6 (wild type) mice. One chip per group with the worst staining results was discarded, resulting in 16 chips total. The chips were combined for the KO and the C57 group, resulting in 2 big virtual chips. The data of the KO chips was compared to the C57 chips. The expression of genetic markers in the knockout chips was compared to expression of the genetic markers in the C57 chips. The resulting data is set forth infra.

Cloning of the Alpha-4 cDNA

The alpha-4 cDNA was PCR amplified in two pieces and subcloned several times before the full-length cDNA was assembled. All PCR reactions were carried out with the “Expand high fidelity PCR kit” and the instructions described therein (Roche-Boehringer Mannheim). As the template DNA a “mouse skeletal muscle 5′ stretch plus cDNA library” (Clontech) was used.

The first, 2.6 kb piece of the alpha-4 cDNA spans exon 1 to exon 23. The primers for this piece were fV and cDNA1B-R. The forward primer contains a MscI restriction site (blunt), the reverse primer is located 3′ of an internal KpnI restriction site. The reverse primer was designed according to [DeMeirsman et al., 1994]. The 2.6 PCR product was extracted from an agarose gel and digested with MluNI (MscI) and KpnI. The fragment was ligated into pBluescript SK—(Stratagene) which was digested with KpnI/SmaI (blunt). The resulting plasmid was named pBSK2.6.

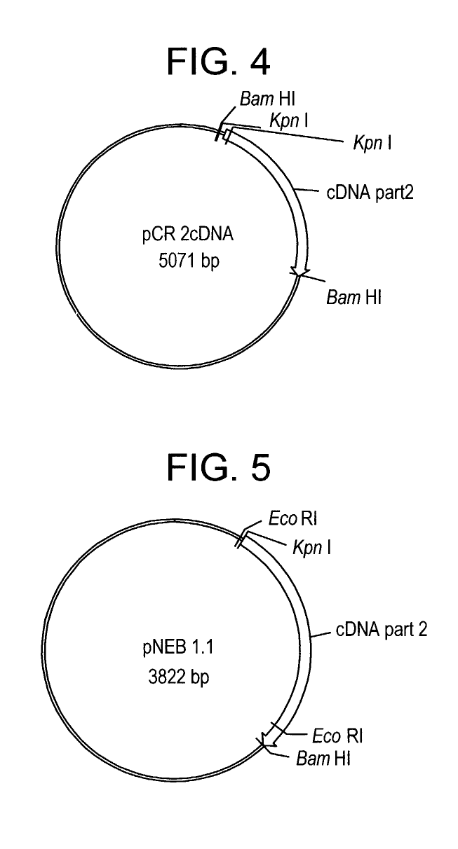

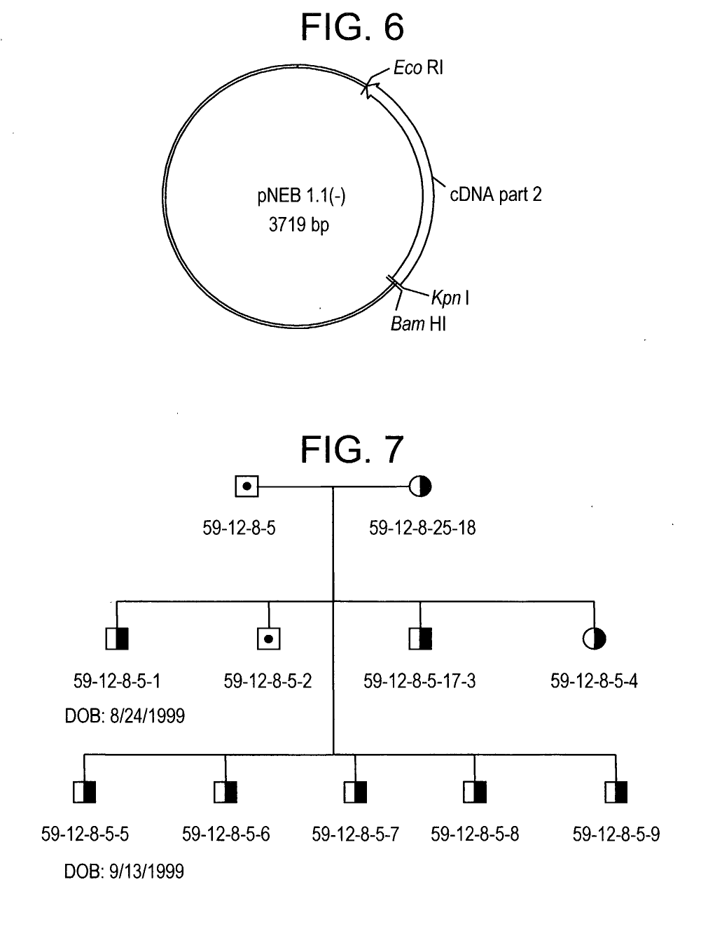

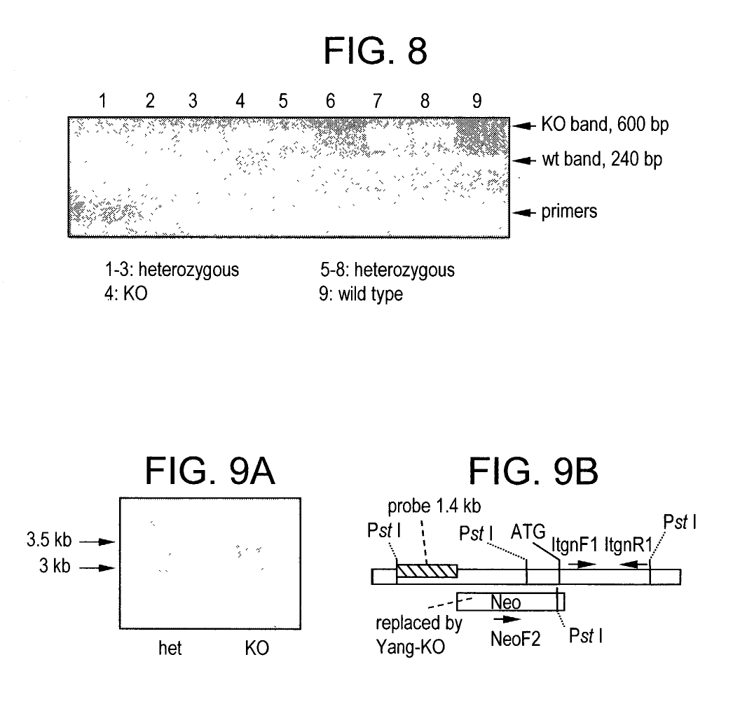

The second, 1.1 kb piece of the cDNA spans exon 23 to a region 5′ of the polyA signals. The primers for this piece were cDNA2-F and cDNA2-R. Both primers were previously published in [DeMeirsman et al., 1994]. The forward primer is located 5′ of an internal KpnI site, the reverse primer introduces a unique BamH site. The resulting 1.1 kb PCR product was cloned into pCR2.1 by following the instructions of the “TA-cloning kit” (Invitrogen). The resulting plasmid was named pCR2cDNA (FIG. 4).

The insert from pCR2cDNA was excised with a KpnI/BamHI restriction digest. This fragment was cloned into pNEB 193, digested as well with KpnI/BamHI. The resulting plasmid was named pNEB1.1 (FIG. 5).

Because the orientation of the cDNA fragment in that pNEB1.1 was such that the missing first piece of the cDNA could not immediately be added, the cDNA insert was excised after digesting pNEB1.1 with KpnI/EcoRI. This fragment was cloned into pNEB193, which was also cut with KpnI/EcoRI. The resulting plasmid was named pNEB1.1(−) (FIG. 6).

In order to combine the first and the second part of the cDNA pieces, pBSK2.6 was digested with BamHI/KpnI. The 2.6 kb piece was ligated into pNEB1.1 (−), digested with the same enzymes. The final plasmid was named pNEB3.6(−) and contained the full-length alpha-4 cDNA, starting ˜65 bp 5′ of the start codon ATG and ending ˜500 bp 3′ of the stop codon TGA. The full-length cDNA can be excised from this plasmid by using BamHI or SalI at the 5′ end and EcoRI at the 3′ end to yield a 3.6 kb piece or with BamHI or SalI at the 5′ end and XmnI at the 3′ end to yield a 3.2 kb piece without the polyA signals in all cases.

Both strands of this plasmid were sequenced by the “primer walking method” by TOPLAB GmbH (Martiensried, Germany). Through comparison to the published cDNA sequence by [Neuhaus et al., 1991] it was found that the start codon postulated by Neuhaus et al. is in fact further downstream, resulting in a signal peptide that is only 33 amino acids instead of 40. This data corresponds exactly to the data published by DeMeirsman et al. in [DeMeirsman et al., 1994].

Insertion of Transgene into an Embryo Taken from an Alpha-4 Heterozygous Knockout Mouse Preparation of Constructs for Transfections and Microinjections

The DNA clone for microinjection (tet-VLA) is cleaved with appropriate enzymes, such as XhoI and NotI and the DNA fragments electrophoresed on 1% agarose gels in TBE buffer (Maniatis et al., 1989). The DNA bands are visualized by staining with ethidium bromide, excised, and placed in dialysis bags containing 0.3 M sodium acetate, pH 7.0. DNA is electroeluted into the dialysis bags, extracted with phenol-choloroform (1:1), and precipitated by two volumes of ethanol. The DNA is redissolved in 1 ml of low salt buffer (0.2 M NaCl, 20 mM TRIS, pH 7.4 and 1 mM EDTA) and purified on an ELUTIP-D column. The column is first primed with 3 ml of high salt buffer (1 M NaCl, 20 mM Tris TM, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml of high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris TM, pH 7.4 and 0.1 mM EDTA. Other methods for purification of DNA for microinjection are also described in Hogan et al., Manipulating the mouse embryo (Cold Spring Harbor Laboratory, Cold Spring Harbor, N.Y. (1986), in Palmiter et al., Nature 300, 611 (1982), in “The Qiagenologist, Application Protocols”, 3^{rd} edition, published by Qiagen, Inc, Chatsworth, Calif., and in Maniatis et al., Molecular Cloning: a laboratory manual (Cold Spring Harbor Laboratory, Cold Spring Harbor, N.Y. 1989), all of which are hereby incorporated by reference herein in their entireties.

Construction of Transgenic Animals:

Animals suitable for transgenic experiments can be obtained from standard commercial sources such as Charles River (Wilmington, Mass.), Taconic (Germantown, N.Y.), Harlan Sprague Dawley (Indianapolis, Ind.), Jackson Laboratories (Bar Harbor, Me.), etc. Swiss Webster female mice are preferred for embryo retrieval and transfer. B6D2F₁ males can be used for mating and vasectomized Swiss Webster studs can be used to stimulate pseudopregnancy. Vasectomized males can be obtained from the supplier.

Microinjection Procedures:

The procedures for manipulation of the rodent embryo and for microinjection of DNA are described in detail in (Hogan et al., “Manipulating the mouse embryo”, Cold Spring Harbor Laboratory, Cold Spring Harbor, N.Y. (1986)), which is hereby incorporated by reference herein in its entirety.

Transgenic Mice

Female mice six weeks of age are induced to superovulate with a 5 IU injection (0.1 cc, ip) of pregnant mare serum gonadotropin (PMSG, Sigma) followed 48 hours later by a 5 IU injection (0.1 cc, ip) of human chorionic gonadotropin (hCG, Sigma). Females are placed with males immediately after hCG injection. Twenty-one hours after hCG, the mated females are sacrificed by CO₂ asphyxiation or cervical dislocation and embryos are recovered from excised oviducts and placed in Dulbecco's phosphate buffered saline with 0.5% bovine serum albumin (BSA, Sigma). Surrounding cumulus cells are removed with hyaluronidase (1 mg/ml). Pronuclear embryos are then washed and placed in Earl's balanced salt solution containing 0.5% BSA (EBSS) in a 37.5° C. incubator with a humidified atmosphere at 5% CO₂, 95% air until the time of injection.

Randomly cycling adult female mice are paired with vasectomized males, Swiss Webster or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS and in the tip of a transfer pipette (about 10-12 embryos). The pipette tip is inserted into the infundibulum and the embryos transferred. After the transfer, the incision is closed by two sutures.

### Results

Breeding Results of Mice.

Line 57: wild type mice.

Line 59: heterozygous x heterozygous alpha-4 KO cross. The 42 matings looked at produced 286 mice as offspring. Out of the 286 mice, 147 were genotyped as heterozygous, 132 as wild type and 7 as homozygous knockouts, which makes a ratio of 1 homozygous KO mouse per 40 animals.

Breeding of a Heterozygous x Homozygous Knockout Mouse

Line 59: heterozygous x homozygous KO cross 59 matings were looked at that produced 298 animals total, out of which 244 were genotyped as heterozygous and 54 animals were genotyped as homozygous knockouts, resulting in a ratio of 1 homozygous KO mouse per 6 animals. The average littersize for this kind of mating in this line was determined as 5 pups per litter.

Breeding of Two Homozygous Knockout Mice

The breeding of two homozygous alpha-4 integrin knockout (KO) mice with two copies of the transgene results only in homozygous knockout offspring. The number of live pups however, is significantly altered in comparison to wild type x wild type breedings. A regular wt x wt breeding pair can have litters of up to 12 pups, which can all be alive and well. The average littersize of a “normal” breeding pair is 6-8 pups per litter. The maximum number of alive pups however for a homozygous KO x homozygous KO cross was:

5 for line 59 (which was an exception to the rule, the average littersize was only 2-3 animals per litter—31 animals produced with 11 matings)

### Genotypic and Phenotypic Evaluation of Knockout Mice of the Present Invention that are Unable to Express Functional Alpha-4 Integrin Protein

After formation of mice that are unable to express functional alpha-4 integrin, the mice were evaluated to determine the genotypic and phenotypic effects of a lack of functional alpha-4 integrin on the mice.

Genotype Analysis

The genomic tail DNA from the animals was tested for the presence of the transgene and also for the background of the endogenous alpha-4 integrin by Southern blotting and/or PCR analysis.

The detection of the transgene was solely done by PCR with specific primers and conditions that only amplify transgenic and not genomic DNA. The reverse primer for the detection of the tetP-portion of the alpha-4 integrin cDNA construct anneals in the second exon of the alpha-4 integrin and only the forward primer (tetpT) is specific for the transgene and anneals in the tet-promoter.

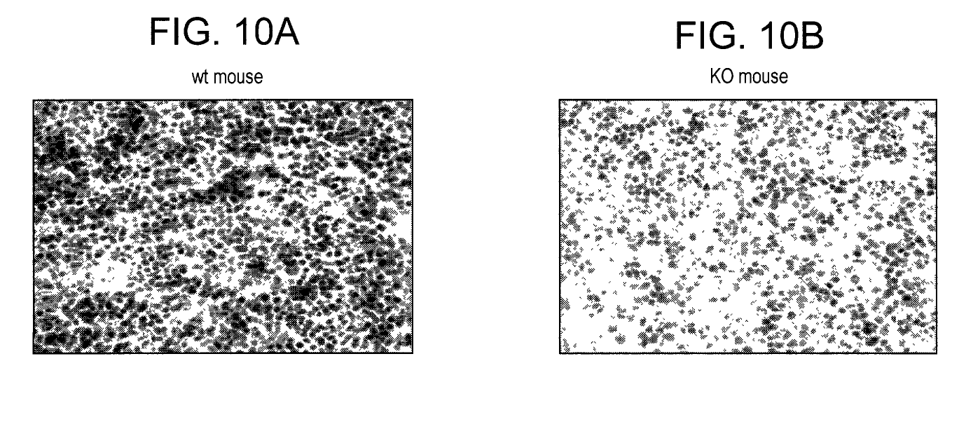

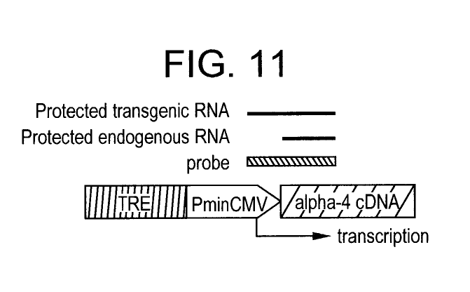

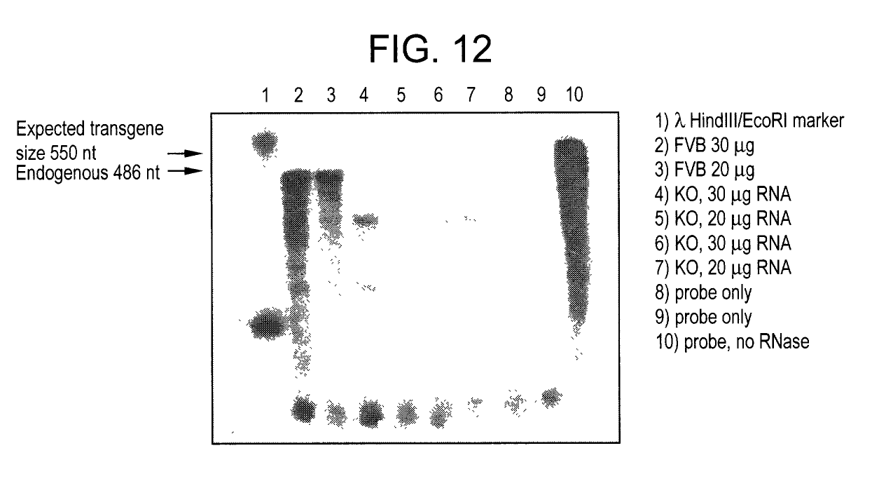

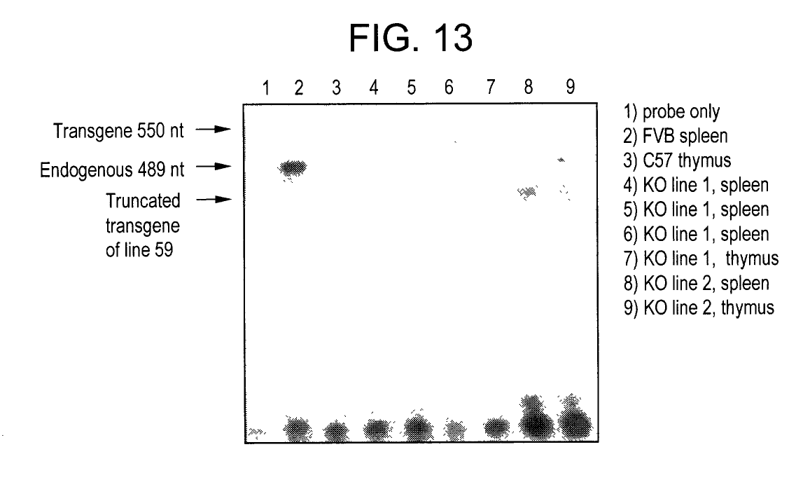

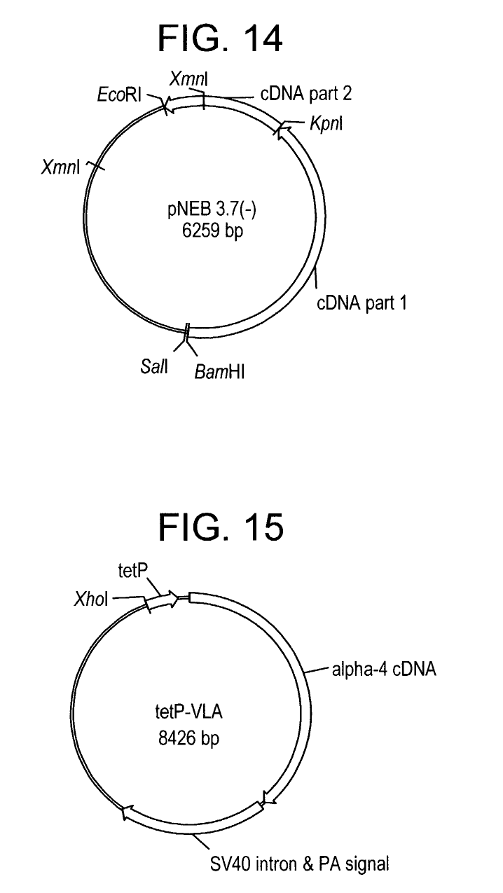

The PCR analysis to assess, whether the endogenous alpha-4 integrin was present as a wt, heterozygous or homozygous knockout, was done with a combination of three primers, two forward primers and one reverse primer. One forward primer (ItgnF1) annealed about 20 bp 5′ of the start codon. This primer only anneals in the genomic, endogenous DNA, since the area was replaced by a neomycin cassette by Yang et al. to generate the heterozygous alpha-4 knockout mice [Yang et al., 1995]. The reverse primer (ItgnR1) binds 3′ of the neo insertion and binds therefore in all occasions. The second forward primer (NeoF2) anneals in the neo cassette, therefore only binding to a targeted allele (FIG. 11(B)). Primers ItgnF1 and ItgnR1 pair in wild type and heterozygotes to produce a ˜240 bp band. Primers NeoF2 and ItgnR1 pair in heterozygotes and homozygotes to produce a ˜600 bp band (FIG. 10).

The southern blot analysis graphically shown in FIG. 10 was done with a 1.4 kb PstI/KpnI probe and restriction digest of the genomic tail DNA with PstI as described in [Yang et al., 1995], yielding a 3.0 kb fragment for the wt allele and a 3.5 kb fragment for the targeted allele.

Phenotype Analysis

Phenotype Analysis of the Overexpression Knockout Mice

One line out of the three generated overexpression knockout lines exhibited significant symptomatic changes around the eyes: the eyes of almost all the knockout mice in line 59 appeared to be infected, whereas heterozygous or wild type littermates, housed in the same or separate cages, did not suffer from the same kind of infection.

However not all of the knockout mice of line 59 had this eye-infection, only a subline, almost all offspring from the first homozygous knockout mouse generated in this line: 59-12-8-5. In order to find out more about the type of infection, two homozygous knockout littermates, born and grown up in the same cage, one with and one without visibly infected eyes were sent for serology, bacteriology and pathology testing to Charles River Laboratories (Wilmington, Mass.).

Both mice were tested negative for serology (19 different ELISA tests performed with both samples).

The conjunctiva of both mice was submitted to a bacteriology test and both mice, also the visibly non-infected one, tested positive for Pasteurella pneumotropica. Both cultures had only very few colonies.

The parasitology reports for both mice were negative (test for 4 different parasites).

Concentration of Progenitor Stem Cells in a Mouse of the Present Invention that does not Express Functional Alpha-4 Integrin Protein

Histopathological analysis of a homozygous alpha-4 integrin knockout mice of the present invention in comparison to wild type (wt) mice showed a marked bone marrow progenitor cell margination into the lungs (2/10 animals with one animal showing venular distention/collapse and increased bone marrow cellularity (mainly granulocytic) with enhanced margination from stromal space (7/10 animals). The spleen of the KO mice of the present invention showed increased extra-medullary haematopoiesis and an increase in germinal center size/cellularity (4/10 animals). Further histopathological findings noted in the homozygous alpha-4 knockout mice of the present invention splenic megakaryocytosis (3/10), splenic erythrophagocytosis with haemosiderosis (1/10 animals) and splenic germinal centre necrosis (2/10 animals). In the jejunum the findings in the homozygous KO mice of the present invention included inflammatory infiltrates in base or body of lamina propria (4/10 animals). In addition one sample of heart muscle from one homozygous alpha-4 knockout animal with bone marrow progenitor cells in the lung with venular collapse showed histological signs of cardiomyopathy.

Immunohistochemistry Analysis Results:

No alpha-4 protein could be detected in the spleen of the knockout mice by IHC, whereas the spleens of wild type mice of the same background stain for the presence of the alpha-4 protein. These results are graphically shown in FIG. 10.

Affymetrix Analysis

The data analysis was generated using an Affymetrix Data Mining Tool. The data of 4 C57 and 4 homozygous alpha-4 integrin KO mice of the present invention were pooled per treatment group.

By comparing the data of the homozygous alpha-4 integrin KO mice of the present invention to the C57 (wt) mice, several genes turned out to be significantly up- or downregulated in the KO mice. Furthermore, there are a number of genes that are downregulated in the homozygous alpha-4 integrin KO group relative to the levels of these genes measured in mice of the C57 line. The tables below list genetic markers whose levels were modulated in the knockout mouse of the present invention, relative to the levels of these same genetic markers in the C57 (wild type mice). A number greater than 1 in the farthest right column of the tables indicates that the expression (and thus, the level) of the genetic marker was up regulated relative to the expression of the genetic marker a wild type mouse, while a number less than 1 in the farthest right column of the tables indicates that expression of the genetic marker was down regulated relative to the expression of the genetic marker in a wild type mouse. Also disclosed in the left column of the tables are the accession numbers for these markers.

Genetic Markers upregulated in the KO mice of the present invention in comparison to their levels measured in C57 (wild type) mice. Fold changes and gene bank accession numbers are indicated.

| | | fold |
|---|---|---|
| Acc. # | | changes |
| ET62762 | Mus musculus anti-von Willebrand factor antibody NMC-4 kappa chain mRN | 3.668 |
| J00475 | Mouse gene for immunoglobulin alpha heavy chain, switch region and con | 2.359 |
| ET61563 | (H-2 CLASS I HISTOCOMPATIBILITY ANTIGEN, D-K ALPHA CHAIN PRECURSOR | 2.112 |
| L00606 | Mus musculus MHC class I Qa-1a antigen mRNA, complete cds. | 4.726 |
| U93862 | Mus musculus ribosomal protein L41 mRNA, complete cds. | 3.653 |
| M27034 | Mouse MHC class I D-region cell surface antigen (D2d) gene, complete cds | 3.398 |
| AJ007909 | Mus musculus mRNA for erythroid differentiation regulator, partial | 3.317 |
| AC005817 | NRNT(1e-92): , complete sequence [Mus musculus] | 2.167 |
| AA415028 | vc50e11.r1 Knowles Solter mouse 2 cell Mus musculus cDNA clone 778028 | 1.791 |
| AJ007971 | NRNT(0.0): Mus musculus mRNA for IIGP protein | 1.445 |
| D78343 | Mouse DNA for Ig gamma-chain, secrete-type and membrane-bound, partial | 1.431 |
| AB003306 | NRNT(2e-61). Mus musculus DNA for PSMB5, complete cds | 5.231 |
| AA168767 | Homologous to sp P32507: POLIOVIRUS RECEPTOR HOMOLOG PRECURSOR. | 5.124 |
| M60429 | Mouse Ig rearranged H-chain mRNA constant region. | 5.003 |
| X64550 | M. musculus mRNA RHAMM | 4.57 |
| R74638 | R74636 MDB0793 Mouse brain, Stratagene Mus musculus cDNA 3′end. | 4.248 |
| AF003867 | Mus musculus pale ear (ep mutant allele) mRNA, partial cds. | 3.457 |
| AA049597 | mj35h09.r1 Soares mouse embryo NbME13.5 14.5 Mus musculus cDNA clone 4 | 3.394 |
| D19392 | MUSGS00761 Mouse 3′-directed cDNA; MUSGS00761; clone mb1494. TIGR clus | 3.389 |
| AA072214 | Homologous to sp P41725: BRAIN ENRICHED HYALURONAN BINDING PROTEIN F | 3.338 |
| X55674 | M. musculus mRNA for D2A dopamine receptor | 3.18 |
| AA111465 | mo54b05.r1 Life Tech mouse embryo 10 5dpc 10665016 Mus musculus cDNA cds | 3.158 |
| AA177433 | mt23g11.r1 Soares mouse 3NbMS Mus musculus cDNA clone 621956 5′ TIGR cds | 1.476 |
| D11468 | Mouse gene for immunoglobulin alpha heavy chain, switch region and con | 5.669 |
| U77415 | Mus musculus Bop1 mRNA, complete cds | 1.606 |
| C75959 | C75959 Mouse 3.5-dpc blastocyst cDNA Mus musculus cDNA clone J0001C05 | 1.43 |

Genetic Markers downregulated in the KO mice of the present invention in comparison to their levels measured in C57 (wild type) mice.

Fold changes and gene bank accession numbers are indicated

| | | fold |
|---|---|---|
| acc. # | | change |
| AA571535 | vm06f11.r1 Knowles Solter mouse blastocyst B1 Mus musculus cDNA clone | 0.341 |
| AF027865 | Mus musculus Major Histocompatibility Locus class II region. | 0.227 |
| U10406 | Mus musculus capping protein beta-subunit isoform 1 mRNA, complete cds | 0.248 |
| AJ006341 | Mus musculus mRNA for peroxisomal integral membrane protein PMP34 | 0.292 |
| AB000677 | Mus musculus mRNA for JAB, complete cds. | 0.889 |
| M21065 | Mouse interferon regulatory factor 1 mRNA, complete cds. | 0.835 |
| U53219 | Mus musculus GTPase IGTP mRNA, complete cds. | 0.85 |
| M64085 | Mouse spi2 proteinase inhibitor (spi2/eb1) mRNA, 3 end | 0.663 |
| AA153021 | Homologous to sp Q01514: INTERFERON-INDUCED GUANYLATE-BINDING PROT | 0.912 |
| AA154371 | Homologous to sp P13765: HLA CLASS II HISTOCOMPATIBILITY ANTIGEN, DO B | 0.327 |
| U45975 | NRNT(3e-39): Human phosphatidylinositol (4,5)bisphosphate 5-phosphatas | 0.218 |
| L38444 | Mus musculus (clone U2) T-cell specific protein mRNA, complete cds. | 0.541 |
| Z11974 | Mus musculus mRNA for macrophage mannose receptor | 0.269 |

By looking at the data generated by each individual mouse and treatmnent group, it was shown that the Affymetrix Data Mining Tool called the murine alpha-4 integrin gene “absent” in every single homozygous knockout mouse of the present invention, whereas the murine alpha-4 integrin was called “present” in all cases of the C57 mice.

### Conclusion

As set forth above, disclosed herein is a novel, useful, and heretofore unknown mouse that survives gestation to mature into a mouse, but is unable to express functional alpha-4 integrin protein. As explained above, such a mouse readily has applications in methods for assaying compounds or agents for alpha-4 integrin antagonist activity.

In addition, it has been discovered that a mouse of the present invention possesses a phenotype that is unique with respect to the phenotype of a wild type mouse. Information gleamed from the phenotype of a mouse of the present invention can readily be used in a method of assaying compounds or agents for their ability to modulate, and particularly to antagonize the signaling activity of VLA-4 receptor protein or the activity of the alpha-4 integrin protein. Compounds or agents possessing such activity may have valuable applications in treating a large variety of inflammatory and autoimmune diseases.

### EXAMPLE II

### Method for Using a Genetic Marker for Evaluating Efficacy of Compounds or Agents in Modulation VLA-4 Receptor Signaling

Using information obtained from a knockout mouse of the present invention as described in Example I, it has been discovered that the level of genetic markers measured within bodily samples of the mouse are modulated relative to the level of these same genetic markers measured in a wildtype mice. Thus, these genetic markers are “surrogate” genetic markers that have immediate applications in evaluating the ability of compounds or agents to modulate signaling activity of VLA-4 receptor. Consequently, the efficacy of such compounds or agents as therapeutic agents for modulating, and particularly antagonizing the signaling activity of VLA-4, and for treating a plethora of diseases or disorders, can be evaluated in a research or clinical setting.

Materials and Methods

Experimental Design:

The following experiments were conducted in order to verify the Affymetrix data described in Example I, and to validate selected genes as surrogate genetic markers for VLA-4 inhibition.

Administration of Known VLA-4 Receptor Antagonists

In these experiments, EAE (experimental allergic encephalomyelitis) mice were used. The EAE mouse is an animal model for Central Nervous System (CNS) autoimmune disease. It is widely used as a human Multiple Sclerosis (MS) model.

EAE mice (see protocol below) vehicle only treated and treated with IVL984 or HMR1031 for 14 days (5 mice per group) were sacrificed by cervical dislocation. The brain was aseptically removed and the RNA was prepared using a standard Trizol (Invitrogen) prep (protocol see below). The prepped RNA was run on an agarose gel to determine the quality of the RNA and quantified by UV spec analysis. Taqman analysis was performed using sequence specific primers and probes (sequence see below).

Animals:

SJL/J female mice, 8 wks. old, (Jackson Laboratories, Bar Harbor, Me.)

Antigens:

Myelin Proteolipid Protein (PLP 139-151) (HSLGKWLGHPDKF (SEQ ID NO: 14)) (Cat #H-2478) BACHEM, Bioscience, Inc., 3700 Horizon Dr., King of Prussia, Pa. 19406. Complete Freund's Adjuvant H37 Ra [1 mg/ml Mycobacterium Tuberculosis H37 Ra] Difco (Cat #3114-60-5, 6×10 ml).

Mycobacterium Tuberculosis Difco, (Cat #3114-33-8, 6×100 mg) Pertussis Toxin.

Bordetella Pertussis (Lyophilized powder containing PBS and lactose) List Biological Laboratories (Product #180)

Induction of EAE in Mice

PLP139-151 peptide is dissolved in H₂O:PBS (1:1) solution to a concentration 5 mg/10 ml (for 50 ug PLP per mouse) or 7.5 mg/10 ml (for 75 ug PLP per group) and emulsified with an equal volume of CFA supplemented with 40 mg/10 ml heated-killed mycobacterium tuberculosis H37Ra. Mice are injected s.c. with 0.2 ml of peptide emulsion in the abdominal flank (0.1 ml on each side). On the same day and 72 hr later, mice are injected i.v. with 100 μl of 35 ng and 50 ng of Bordetella Pertussis toxin in saline respectively.

Treatment IVL984 or HMR1031 or vehicle control only: 0.2% Hydroxypropyl Methylcellulose) started 7 days after immunization, before the first EAE symptoms appeared.

EAE mice, vehicle

N=10 (5 mice were used for Taqman)

EAE mice treated with HMR1031A, 50 mg/kg, q.d., s.c. for 14 days from day 7

N=10 (5 mice were used for Taqman)

EAE mice treated with IVL984 50 mg/kg, q.d., s.c. for 14 days from day 7

N=10 (5 mice were used for Taqman)

TRIzol (Invitrogen) RNA Prep:

Tissue Homogonezation:

Brain was divided into two halves and each half was placed in a steril 1.5 ml tube. 0.5 ml TRIzol was added to each tube and the tissue was homogenized using a hand held tissue homogenizer. After homogenization, another 0.5 ml of TRIzol was added to each tube and the samples were incubated for 5 minutes at room temperature to permit complete dissociation of nucleoprotein complexes.

Phase Separation

0.2 ml of Chloroform (Sigma) was added to each tube and vortexed. Samples were incubated for an additional 5 minutes at room temperature and then centrifuged at 12,000×g for 15 minutes at 4° C.

RNA Precipitation

The upper aqueous phase was transferred into a fresh, steril tube and the two sample halves per mouse were combined. 0.5 ml isopropylalcohol was added to each combined sample, vortexed and incubated at room temperature for 10 minutes. The samples were then centrifuged at 12,000×g at 4° C.

RNA Wash

The supernatant was removed and the pellets washed with 1 ml of 75% Ethanol, centrifuged again for 5 minutes at 7,500×g at 4° C.

Redissolving the RNA

The final pellets were briefly air dried and resuspended in nuclease-free, sterile water. An aliquot of the RNA was run on an agarose gel to determine the presence of the 18 and 28 S bands. The concentration was determined by measuring the absorbance at 260 nm.

Taqman:

Taqman primers were ordered for Mus musculus mRNA for macrophage mannose receptor (Accession number: Z11974). (Sequence of the M. musculus mRNA for macrophage mannose receptor: SEQ ID NO 13). Primers for the real time Taqman PCR studies were chosen using Primer Express software (Perkin Elmer) and synthesized by Sigma Genosys. The sequences of the forward and reverse primers were CAATTCACGAGAGGCAGGGA (SEQ ID NO: 15) and GGGAAGGGTCAGTCTGTGTTTG (SEQ ID NO: 16) respectively. PCR product was run on the 4% agarose gel to confirm presence of a single band. PCR reactions were run on ABI Prizm System 7700 sequence detector (Perkin-Elmer) using CybrGreen PCR Core Reagents Kit (Perkin-Elmer) according to the manufacturer's protocol. The optimum final primer concentration in reactions was found to be 0.2 uM. The results were normalized to 18S and expressed as logarithm base 2 of copy number difference with 18S RNA levels. Samples from at least 3 independent RT reactions per point were used.

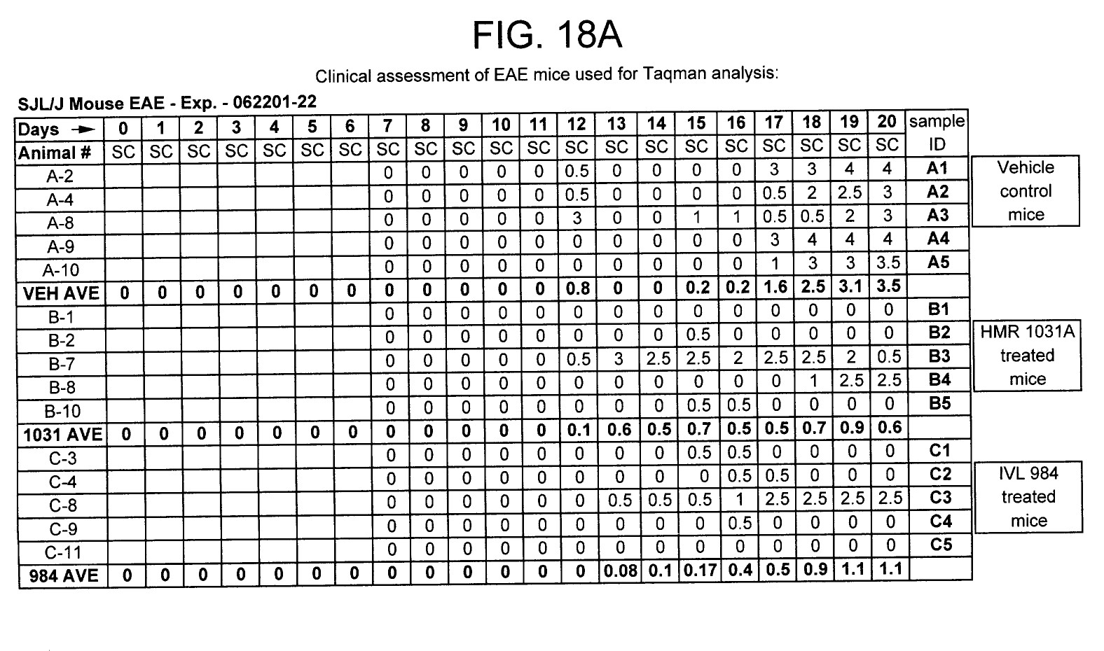

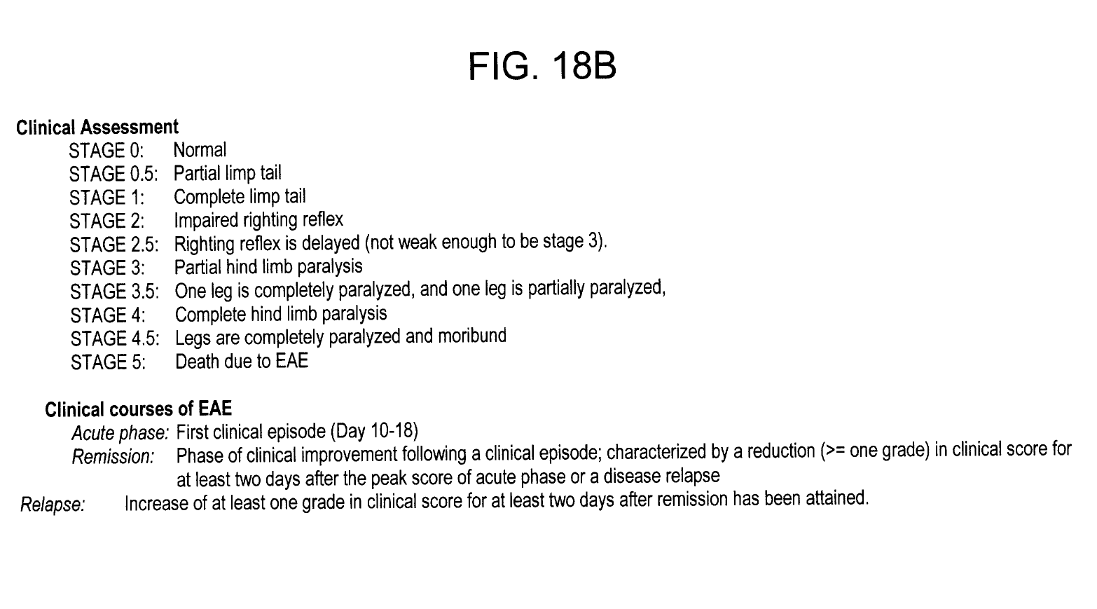

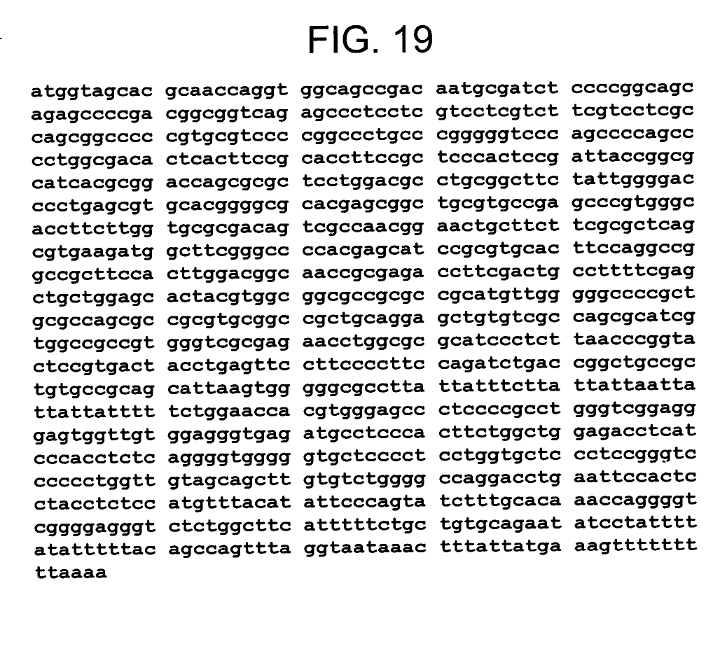

Clinical assessment of EAE mice is described in FIG. 18.

Results

Brain Samples

The mRNA levels of the HMR 1031 and IVL 984 treated EAE mice are statistically significant lower in comparison to the vehicle control mice.

Spleen Samples

The spleen samples do not show the same tendencies as the brain in either treatment (p-value: 0.01-0.05)

The able below is a brief synopsis of the results

| | HMR 1031 | | IVL 984 | |
|---|---|---|---|---|
| Analyzed gene: | Brain | Spleen | Brain | Spleen |
| Macrophage mannose receptor | ✓ | — | Tendency | — |

✓: Statistically significant decrease in macrophage mannose receptor mRNA or protein levels with a 5% or lower probability rate in treated mice compared to vehicle control mice.
Tendency: The decrease in macrophage mannose receptor mRNA levels is a statistically significant, a probability rate of 5-15%.
—: The changes in mRNA levels are statisically significant at a probability rate of higher than 15% (not significant).

These results show that levels of the genetic marker macrophage mannose receptor mRNA are statistically significantly lower in bodily samples taken from EAE mice treated with a VLA-4 receptor antagonist than levels measured in EAE mice not treated with such an antagonist.

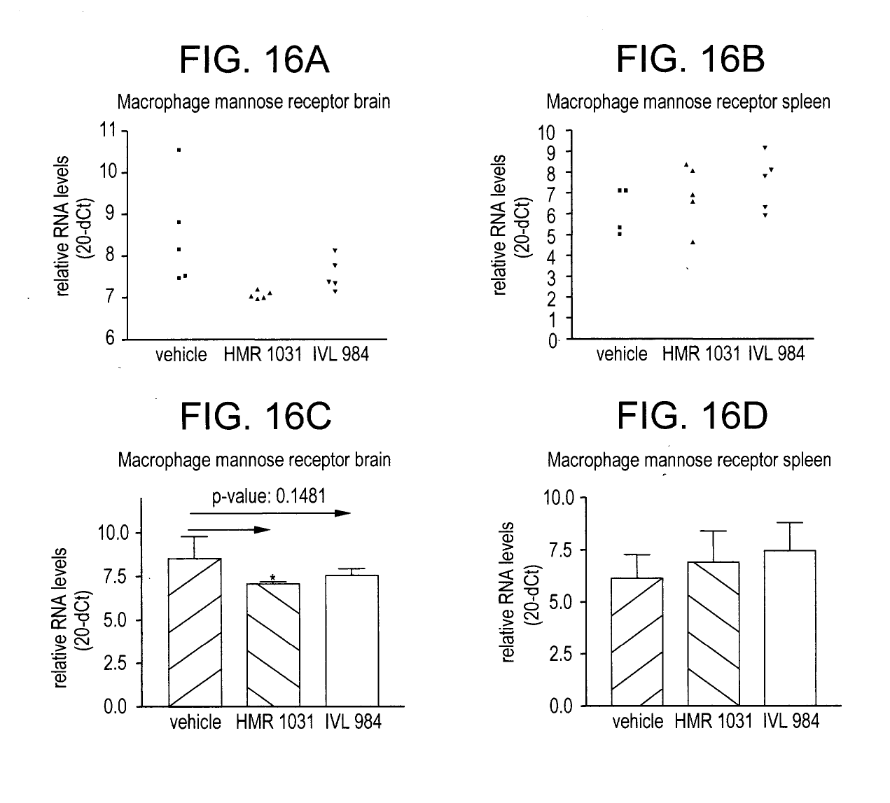

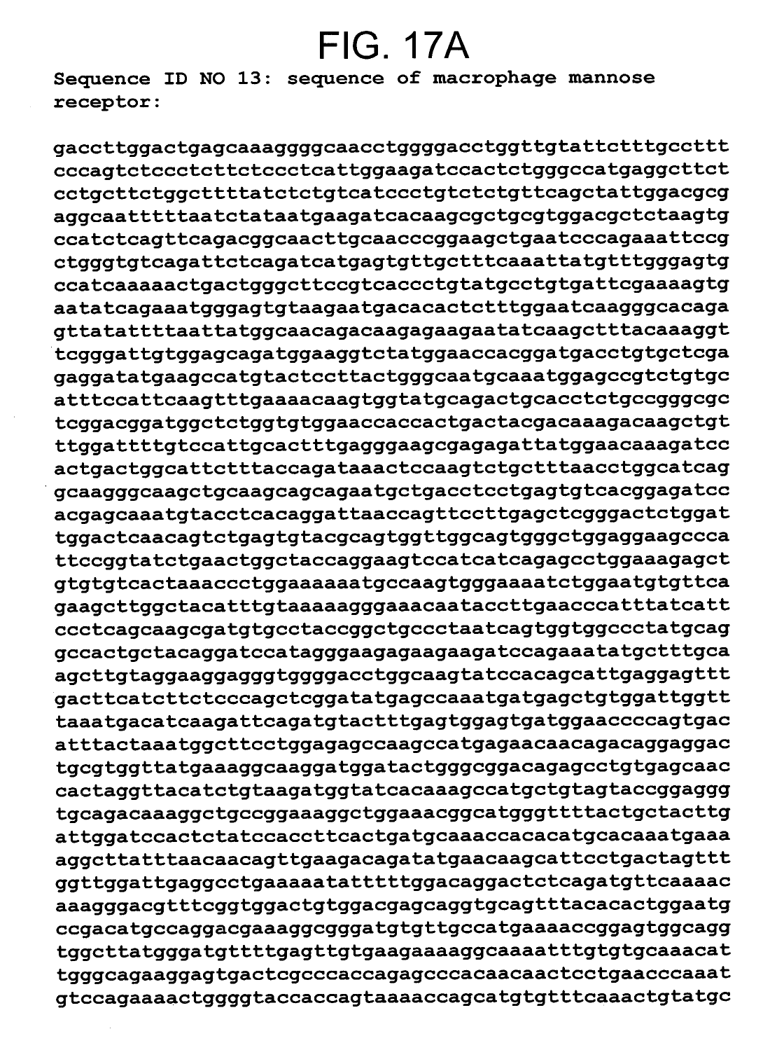

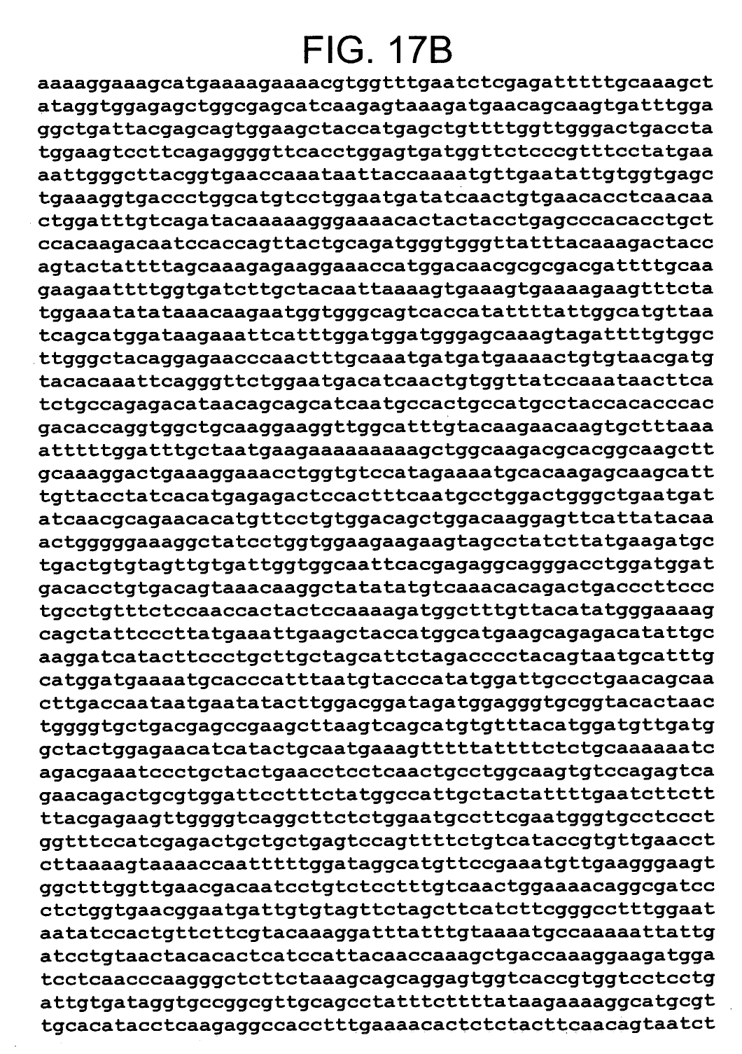

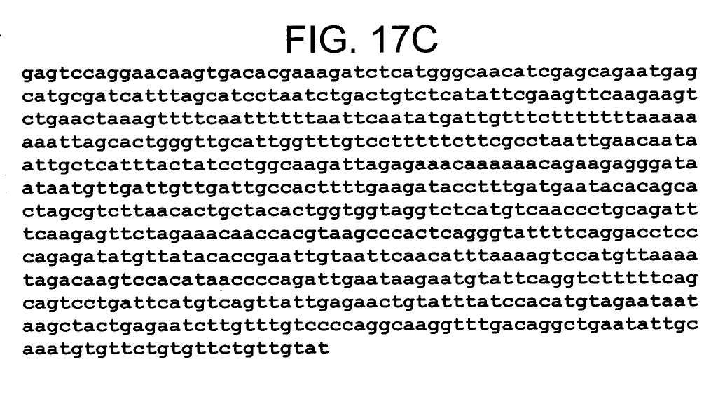

Detailed Taqman results: FIG. 16:

### Discussion

The results set forth above, as well as in FIG. 16, clearly demonstrate that genetic markers discovered to have modulated expression in a knockout mouse of Example I are surrogate genetic markers for the modulation of the signaling activity of VLA-4 receptor. Thus, they can readily be used to determine whether a compound or agent has efficacy in modulating the signaling activity of VLA-4 receptor. In particular, the genetic marker M. musculus mRNA for macrophage mannose receptor, which was determined to have decreased levels of expression in an alpha-4 integrin knockout mice of Example I, also has decreased levels of expression in an organism to which a known VLA-4 receptor antagonist, i.e. IVL 984 or HMR1031, is administered. Hence, methods of the present invention can readily be used to identify antagonists of the signaling activity of VLA-4 receptor, which readily have application in treating a plethera of diseases, including, but certainly not limited to asthma, arthritis, and multiple sclerosis, to name only a few. Moreover, methods of the present invention for determining whether a compound or agent has efficacy in modulating signaling activity of VLA-4 receptor can also readily be used to monitor a patient to whom a VLA-4 antagonist is administered, particularly in a clinical setting.

### EXAMPLE III

### Method for Using a Genetic Marker for Evaluating Efficacy of Compounds or Agents in Modulating VLA-4 Receptor Signaling

Using information obtained from a knockout mouse of the present invention as described in Example I, it has been discovered that the level of genetic markers measured within bodily samples of the mouse are modulated relative to the level of these same genetic markers measured in a wildtype mice. Thus, these genetic markers are “surrogate” genetic markers that have immediate applications in evaluating the ability of compounds or agents to modulate signaling activity of VLA-4 receptor. Consequently, the efficacy of such compounds or agents as therapeutic agents for modulating, and particularly antagonizing the signaling activity of VLA-4, and for treating a plethora of diseases or disorders, can be evaluated in a research or clinical setting.

Materials and Methods

Experimental Design:

The following experiments were conducted in order to verify the Affymetrix data described in Example I, and to validate selected genes as surrogate genetic markers for VLA-4 inhibition.

Administration of Known VLA-4 Receptor Antagonists

In these experiments, EAE (experimental allergic encephalomyelitis) mice as well as KO and wt mice of the present invention were used. The EAE mouse is an animal model for Central Nervous System (CNS) autoimmune disease. It is widely used as a human Multiple Sclerosis (MS) model.

EAE mice (see protocol below) vehicle only treated and treated with IVL984 for 14 days (5 mice per group) as well as 5 alpha-4 integrin KO mice of the present invention and 4 wt mice of the same genetic background were sacrificed by cervical dislocation. The spleen was aseptically removed and placed in a 15 ml centrifuge tube containing RPMI 1640 medium with 1% FCS. The spleens were then individually meshed in a 10 cm petri dish through a sterile wire screen using a sterile rubber policeman (0.23 mm pore size screen, Thomas Scientific). The screen was washed and the cell suspension collected and transferred into a 15 ml centrifuge tube. After centrifugation at 1200 rpm for 10 minutes at 4° C., the supernatant was decanted and the red blood cells lysed with 1 ml/spleen ice-cold red blood cell lysis buffer for 1-3 minutes on ice. The supernatant was then carefully transferred into a fresh 15 ml centrifuge tube, leaving the fat pellet behind. The cell suspension was centrifuged for 10 minutes at 1200 rpm at 4° C. The supernatant was discarded and the cells resuspended in PBS and placed on ice. The number of alive cells was determined using a hemacytometer (Hauser Scientific) and Trypan Blue (Gibco, Life Technologies) as the dye. The cells were diluted to a final concentration of 10⁷ cells/ml. The cells were then stained for the SOCS-1 (C20) and the SOCS-1 (N-18) protein as follows:

1) Prepare a 0.2 μg/μl dilution of both SOCS-1 antibodies (SOCS-1 (C-20), Santa Cruz, sc-7005; SOCS-1 (N-18), Santa Cruz, sc-7006; Flourescein isothiocyanate (FITC) conjugated mouse IgG₁, κ Monoclonal immunoglobulin isotype standard (anti KLH), PharMingen, 03214C) as well as the isotypic control in PBS, aliquot 25 μl of that dilution into wells of a 96-well plate.

2) Add 100 μl cell suspension (10⁷ cells/ml)

3) Tap plate slightly and incubate at 4° C. for 30-60 minutes

4) Add 100 μl PBS to each well and spin at 4° C., 700 rpm for 5 minutes

5) Flip plate to discard the supernatant

6) Add secondary antibody to wells stained with SOCS-1 antibody: donkey anti-goat FITC: dilute 1:100 in PBS and add 100 μl to each well. Add PBS to wells stained with isotypic control

7) Incubate at 4° C. for 30-60 minutes

8) Add 100 μl PBS to each well

9) Spin plate at 4° C. for 5 minutes at 700 rpm Flip plate to discard supernatant

10) Resuspend cells in 0.1% Formaldehyde in PBS and run FACS analysis to determine number of positive cells

11) Red blood cell lysis buffer: 8.29 g NH₄Cl; 0.037 g EDTA; 1 g KHCO₃; Water ad 1 liter, steril-filter.

EAE mice protocol:

Animals:

SJL/J female mice, 8 wks. old, (Jackson Laboratories, Bar Harbor, Me.) Alpha-4 integrin KO and wt mice of the present invention

Antigens:

Myelin Proteolipid Protein (PLP 139-151) (HSLGKWLGHPDKF (SEQ ID NO: 14)) (Cat #H-2478) BACHEM, Bioscience, Inc., 3700 Horizon Dr., King of Prussia, Pa. 19406. Complete Freund's Adjuvant H37 Ra [1 mg/ml Mycobacterium Tuberculosis H37 Ra] Difco (Cat #3114-60-5, 6×10 ml).

Mycobacterium Tuberculosis Difco, (Cat #3114-33-8, 6×10 mg) Pertussis Toxin.

Bordetella Pertussis (Lyophilized powder containing PBS and lactose) List Biological Laboratories (Product #180)

Induction of EAE in Mice

PLP139-151 peptide is dissolved in H₂O:PBS (1:1) solution to a concentration 5 mg/10 ml (for 50 ug PLP per mouse) or 7.5 mg/10 ml (for 75 ug PLP per group) and emulsified with an equal volume of CFA supplemented with 40 mg/10 ml heated-killed mycobacterium tuberculosis H37Ra. Mice are injected s.c. with 0.2 ml of peptide emulsion in the abdominal flank (0.1 ml on each side). On the same day and 72 hr later, mice are injected i.v. with 100 μl of 35 ng and 50 ng of Bordetella Pertussis toxin in saline respectively.

Treatment IVL984 or vehicle control only (0.2% Hydroxypropyl Methylcellulose) started 7 days after immunization, before the first EAE symptoms appeared.

EAE mice, vehicle

N=10 (5 mice were used for FACS)

EAE mice treated with IVL984 50 mg/kg, q.d., s.c. for 14 days from day 7

N=10 (5 mice were used for FACS)

Untreated mice, no EAE induction:

alpha-4 integrin KO mice of the present invention

N=5

alpha-4 integrin wt mice of the present invention

N=4

Results

These results show that levels of the genetic marker JAB or SOCS-1 protein are statistically significantly lower in bodily samples taken from EAE mice treated with a VLA-4 receptor antagonist than levels measured in EAE mice not treated with such an antagonist. The FACS analysis was performed with a C and an N-terminal antibody against JAB and both antibodies show corresponding results. The results also show that JAB is not only downregulated on the RNA level in the KO mice of the present invention in comparison to wt mice, but JAB is also statistically significantly downregulated on the protein level as determined with both antibodies used.

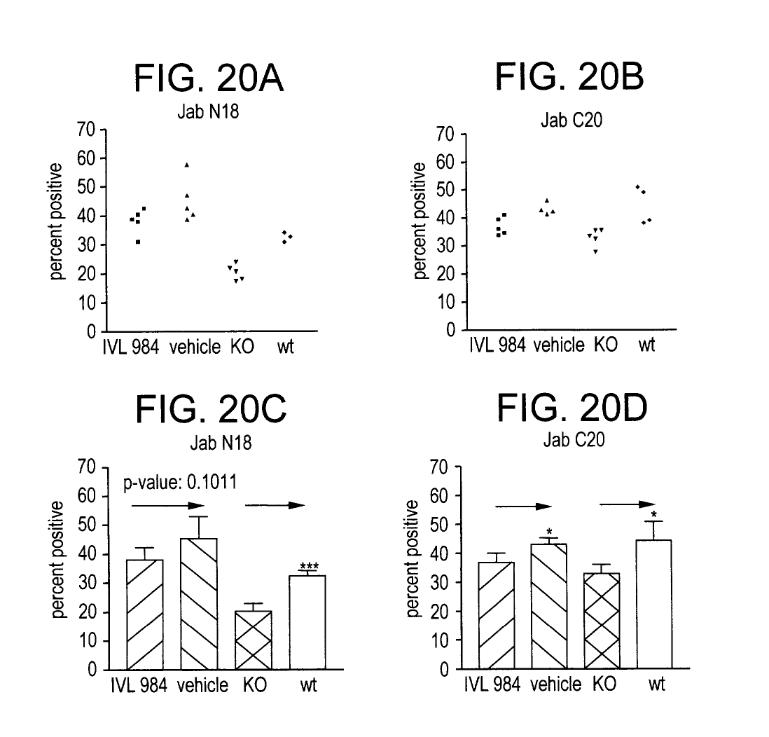

Detailed FACS Results: FIG. 20:

### Discussion

The results set forth above, as well as in FIG. 20, clearly demonstrate that genetic markers discovered to have modulated expression in a knockout mouse of Example I are surrogate genetic markers for the modulation of the signaling activity of VLA-4 receptor. Thus, they can readily be used to determine whether a compound or agent has efficacy in modulating the signaling activity of VLA-4 receptor. In particular, the genetic marker Jab or SOCS-1, which was determined to have decreased levels of expression in an alpha-4 integrin knockout mice of Example I on the RNA level, also has decreased levels of expression on the protein level in an organism to which a known VLA-4 receptor antagonist, i.e. IVL 984, is administered. Hence, methods of the present invention can readily be used to identify antagonists of the signaling activity of VLA-4 receptor, which readily have application in treating a plethera of diseases, including, but certainly not limited to asthma, arthritis, and multiple sclerosis, to name only a few. Moreover, methods of the present invention for determining whether a compound or agent has efficacy in modulating signaling activity of VLA-4 receptor can also readily be used to monitor a patient to whom a VLA-4 antagonist is administered, particularly in a clinical setting.

### EXAMPLE IV

### Method for Using a Genetic Marker for Evaluating Efficacy of Compounds or Agents in Modulating VLA-4 Receptor Signaling

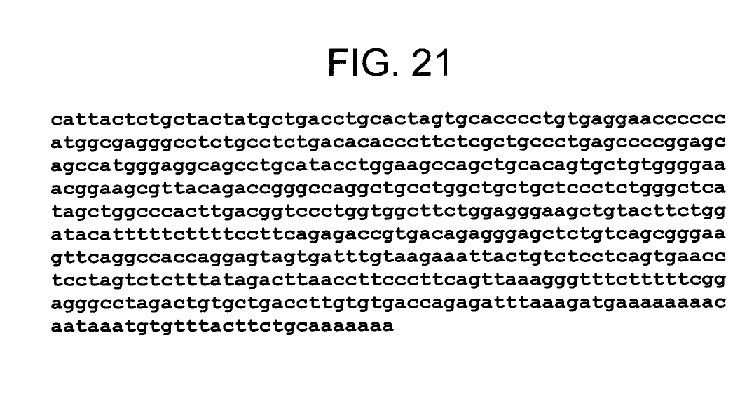

Using information obtained from a knockout mouse of the present invention as described in Example I, it has been discovered that the level of genetic markers measured within bodily samples of the mouse are modulated relative to the level of these same genetic markers measured in a wildtype mice. Thus, these genetic markers are “surrogate” genetic markers that have immediate applications in evaluating the ability of compounds or agents to modulate signaling activity of VLA-4 receptor. Consequently, the efficacy of such compounds or agents as therapeutic agents for modulating, and particularly antagonizing the signaling activity of VLA-4, and for treating a plethora of diseases or disorders, can be evaluated in a research or clinical setting. In this example, the genetic marker EST AA 571535 (vm06f11.r1 Knowles Solter mouse blastocyst B1 Mus musculus cDNA clone) having a DNA sequence of SEQ ID NO: 18) and also shown in FIG. 21, is used. This genetic marker was found in Example I to be downregulated in a knockout mouse of the present invention.

Materials and Methods

Experimental Design:

The following experiments were conducted in order to verify the Affymetrix data described in Example I, and to validate selected genes as surrogate genetic markers for VLA-4 inhibition.

Administration of Known VLA-4 Receptor Antagonists

In these experiments, EAE (experimental allergic encephalomyelitis) mice were used. The EAE mouse is an animal model for Central Nervous System (CNS) autoimmune disease. It is widely used as a human Multiple Sclerosis (MS) model.

EAE mice (see protocol below) vehicle only treated and treated with IVL984 or HMR1031 for 14 days (5 mice per group) were sacrificed by cervical dislocation. The brain was aseptically removed and the RNA was prepared using a standard Trizol (Invitrogen) prep (protocol see below). The prepped RNA was run on an agarose gel to determine the quality of the RNA and quantified by UV spec analysis. Taqman analysis was performed using sequence specific primers and probes (sequence see below).

Animals:

SJL/J female mice, 8 wks. old, (Jackson Laboratories, Bar Harbor, Me.)

Antigens:

Myelin Proteolipid Protein (PLP 139-151) (HSLGKWLGHPDKF (SEQ ID NO: 14)) (Cat #H-2478) BACHEM, Bioscience, Inc., 3700 Horizon Dr., King of Prussia, Pa. 19406. Complete Freund's Adjuvant H37 Ra [1 mg/ml Mycobacterium Tuberculosis H37 Ra] Difco (Cat #3114-60-5, 6×10 ml).

Mycobacterium Tuberculosis Difco, (Cat #3114-33-8, 6×100 mg) Pertussis Toxin.

Bordetella Pertussis (Lyophilized powder containing PBS and lactose) List Biological Laboratories (Product #180)

Induction of EAE in Mice

PLP139-151 peptide is dissolved in H₂O:PBS (1:1) solution to a concentration 5 mg/10 ml (for 50 ug PLP per mouse) or 7.5 mg/10 ml (for 75 ug PLP per group) and emulsified with an equal volume of CFA supplemented with 40 mg/10 ml heated-killed mycobacterium tuberculosis H37Ra. Mice are injected s.c. with 0.2 ml of peptide emulsion in the abdominal flank (0.1 ml on each side). On the same day and 72 hr later, mice are injected i.v. with 100 μl of 35 ng and 50 ng of Bordetella Pertussis toxin in saline respectively.

Treatment IVL984 or HMR1031 or vehicle control only: 0.2% Hydroxypropyl Methylcellulose) started 7 days after immunization, before the first EAE symptoms appeared.

EAE mice, vehicle

N=10 (5 mice were used for Taqman)

EAE mice treated with HMR1031A, 50 mg/kg, q.d., s.c. for 14 days from day 7

N=10 (5 mice were used for Taqman)

EAE mice treated with IVL984 50 mg/kg, q.d., s.c. for 14 days from day 7

N=10 (5 mice were used for Taqman)

TRizol (Invitrogen) RNA Prep:

Tissue Homogenization:

Brain was divided into two halves and each half was placed in a sterile 1.5 ml tube. 0.5 ml TRIzol was added to each tube and the tissue was homogenized using a hand held tissue homogenizer. After homogenization, another 0.5 ml of TRIzol was added to each tube and the samples were incubated for 5 minutes at room temperature to permit complete dissociation of nucleoprotein complexes.

Phase Separation

0.2 ml of Chloroform (Sigma) was added to each tube and vortexed. Samples were incubated for an additional 5 minutes at room temperature and then centrifuged at 12,000×g for 15 minutes at 4° C.

RNA Precipitation

The upper aqueous phase was transferred into a fresh, sterile tube and the two sample halves per mouse were combined. 0.5 ml isopropylalcohol was added to each combined sample, vortexed and incubated at room temperature for 10 minutes. The samples were then centrifuged at 12,000×g at 4° C.

RNA Wash

The supernatant was removed and the pellets washed with 1 ml of 75% Ethanol, centrifuged again for 5 minutes at 7,500×g at 4° C.

Redissolving the RNA

The final pellets were briefly air dried and resuspended in nuclease-free, sterile water. An aliquot of the RNA was run on an agarose gel to determine the presence of the 18 and 28 S bands. The concentration was determined by measuring the absorbance at 260 nm.

Taqman:

Taqman primers were ordered for the EST with the accession number AA571535 (EST AA571535 (sequence for the EST: SEQ ID NO 18). Primers for the real time Taqman PCR studies were chosen using Primer Express software (Perkin Elmer) and synthesized by Sigma Genosys. The sequences of the forward and reverse primers were AGCAGCCATGGGAGGCA (SEQ ID NO: 19) and TCCGTTTCCCCACAGCAC (SEQ ID NO: 20) respectively. PCR product was run on the 4% agarose gel to confirm presence of a single band. PCR reactions were run on ABI Prizm System 7700 sequence detector (Perkin-Elmer) using CybrGreen PCR Core Reagents Kit (Perkin-Elmer) according to the manufacturer's protocol. The optimum final primer concentration in reactions was found to be 0.2 uM. The results were normalized to 18S and expressed as logarithm base 2 of copy number difference with18S RNA levels. Samples from at least 3 independent RT reactions per point were used.

Clinical Assessment of EAE Mice is Described in FIG. 18.

Results

Brain Samples

The mRNA levels of the HMR 1031 and IVL 984 treated EAE mice are statistically significant lower in comparison to the vehicle control mice.

Spleen Samples

The spleen samples do not show the same tendencies as the brain in either treatment (p-value: 0.01-0.05)

The able below is a brief synopsis of the results

| | |
|---|---|
| HMR 1031 | IVL 984 |

| | | | | |
|---|---|---|---|---|
| Analyzed gene: | Brain | Spleen | Brain | Spleen |

| | | | | |
|---|---|---|---|---|
| EST AA 571535 | ✓ | — | — | — |

✓: Statistically significant decrease in macrophage mannose receptor mRNA or protein levels with a 5% or lower probability rate in treated mice compared to vehicle control mice.
—: The changes in mRNA levels are statistically significant at a probability rate of higher than 15% (not significant).

These results show that levels of the genetic marker EST AA571535 are statistically significantly lower in bodily samples taken from EAE mice treated with a VLA-4 receptor antagonist than levels measured in EAE mice not treated with such an antagonist.

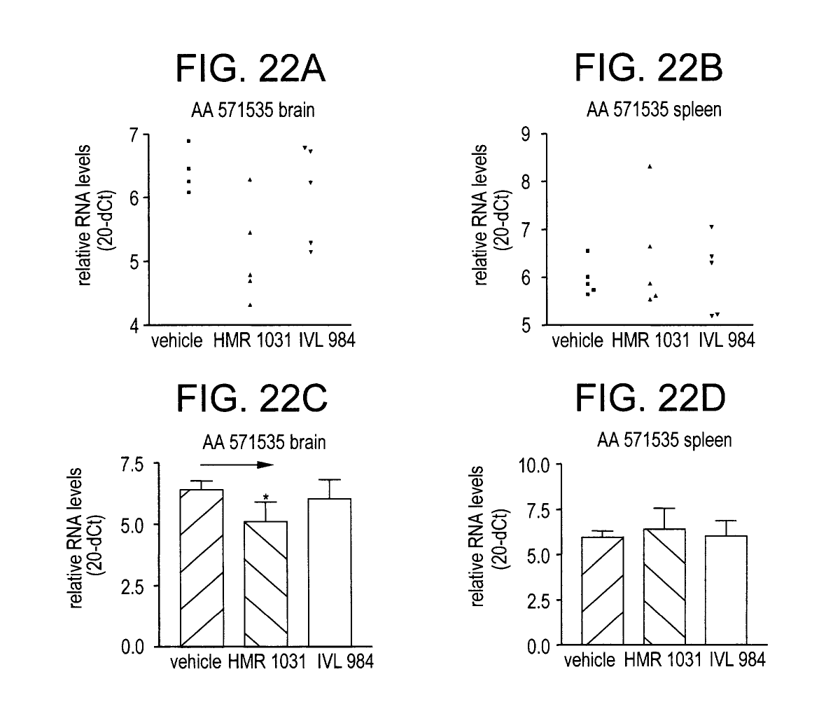

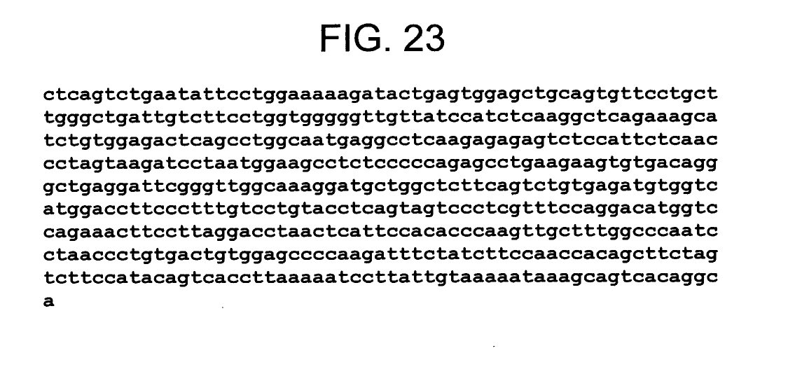

Detailed Taqman Results: FIG. 22.

### Discussion

The results set forth above, as well as in FIG. 22, clearly demonstrate that genetic markers discovered to have modulated expression in a knockout mouse of Example I are surrogate genetic markers for the modulation of the signaling activity of VLA-4 receptor. Thus, they can readily be used to determine whether a compound or agent has efficacy in modulating the signaling activity of VLA-4 receptor. In particular, the genetic marker EST AA571535, which was determined to have decreased levels of expression in an alpha-4 integrin knockout mice of Example I, also has decreased levels of expression in an organism to which a known VLA-4 receptor antagonist, i.e. IVL 984 or HMR1031, is administered. Hence, methods of the present invention can readily be used to identify antagonists of the signaling activity of VLA-4 receptor, which readily have application in treating a plethora of diseases, including, but certainly not limited to asthma, arthritis, and multiple sclerosis, to name only a few. Moreover, methods of the present invention for determining whether a compound or agent has efficacy in modulating signaling activity of VLA-4 receptor can also readily be used to monitor a patient to whom a VLA-4 antagonist is administered, particularly in a clinical setting.

### EXAMPLE V

### Method for Using a Genetic Marker for Evaluating Efficacy of Compounds or Agents in Modulating VLA-4 Receptor Signaling

Using information obtained from a knockout mouse of the present invention as described in Example I, it has been discovered that the level of genetic markers measured within bodily samples of the mouse are modulated relative to the level of these same genetic markers measured in a wildtype mice. Thus, these genetic markers are “surrogate” genetic markers that have immediate applications in evaluating the ability of compounds or agents to modulate signaling activity of VLA-4 receptor. Consequently, the efficacy of such compounds or agents as therapeutic agents for modulating, and particularly antagonizing the signaling activity of VLA-4, and for treating a plethora of diseases or disorders, can be evaluated in a research or clinical setting. In this example, the genetic marker AA154371 (Homologous to sp P13765: HLA CLASS II histocompatibility antigen, DO B) having the DNA sequence of SEQ ID NO: 21 (FIG. 21) is used. In example I, this genetic marker was determined to be downregulated in a knockout mouse of the present invention.

Materials and Methods

Experimental Design:

The following experiments were conducted in order to verify the Affymetrix data described in Example I, and to validate selected genes as surrogate genetic markers for VLA-4 inhibition.

Administration of Known VLA-4 Receptor Antagonists

In these experiments, EAE (experimental allergic encephalomyelitis) mice were used. The EAE mouse is an animal model for Central Nervous System (CNS) autoimmune disease. It is widely used as a human Multiple Sclerosis (MS) model.

EAE mice (see protocol below) vehicle only treated and treated with IVL984 or HMR1031 for 14 days (5 mice per group) were sacrificed by cervical dislocation. The brain was aseptically removed and the RNA was prepared using a standard Trizol (Invitrogen) prep (protocol see below). The prepped RNA was run on an agarose gel to determine the quality of the RNA and quantified by UV spec analysis. Taqman analysis was performed using sequence specific primers and probes (sequence see below).

Animals:

SJL/J female mice, 8 wks. old, (Jackson Laboratories, Bar Harbor, Me.)

Antigens:

Myelin Proteolipid Protein (PLP 139-151) (HSLGKWLGHPDKF (SEQ ID NO: 14)) (Cat #H-2478) BACHEM, Bioscience, Inc., 3700 Horizon Dr., King of Prussia, Pa. 19406. Complete Freund's Adjuvant H37 Ra [1 mg/ml Mycobacterium Tuberculosis H37 Ra] Difco (Cat #3114-60-5, 6×10 ml).

Mycobacterium Tuberculosis Difco, (Cat #3114-33-8, 6×100 mg) Pertussis Toxin.

Bordetella Pertussis (Lyophilized powder containing PBS and lactose) List Biological Laboratories (Product #180)

Induction of EAE in Mice

PLP139-151 peptide is dissolved in H₂O:PBS (1:1) solution to a concentration 5 mg/10 ml (for 50 ug PLP per mouse) or 7.5 mg/10 ml (for 75 ug PLP per group) and emulsified with an equal volume of CFA supplemented with 40 mg/10 ml heated-killed mycobacterium tuberculosis H37Ra. Mice are injected s.c. with 0.2 ml of peptide emulsion in the abdominal flank (0.1 ml on each side). On the same day and 72 hr later, mice are injected i.v. with 100 μl of 35 ng and 50 ng of Bordetella Pertussis toxin in saline respectively. Treatment IVL984 or HMR1031 or vehicle control only: 0.2% Hydroxypropyl Methylcellulose) started 7 days after immunization, before the first EAE symptoms appeared.

EAE mice, vehicle

N=10 (5 mice were used for Taqman)

EAE mice treated with HMR1031A, 50 mg/kg, q.d., s.c. for 14 days from day 7

N=10 (5 mice were used for Taqman)

EAE mice treated with IVL984 50 mg/kg, q.d., s.c. for 14 days from day 7

N=10 (5 mice were used for Taqman)

TRIzol (Invitroyen) RNA Prep:

Tissue Homogenization:

Brain was divided into two halves and each half was placed in a sterile 1.5 ml tube. 0.5 ml TRIzol was added to each tube and the tissue was homogenized using a hand held tissue homogenizer. After homogenization, another 0.5 ml of TRIzol was added to each tube and the samples were incubated for 5 minutes at room temperature to permit complete dissociation of nucleoprotein complexes.

Phase Separation

0.2 ml of Chloroform (Sigma) was added to each tube and vortexed. Samples were incubated for an additional 5 minutes at room temperature and then centrifuged at 12,000×g for 15 minutes at 4° C.

RNA Precipitation

The upper aqueous phase was transferred into a fresh, sterile tube and the two sample halves per mouse were combined. 0.5 ml isopropylalcohol was added to each combined sample, vortexed and incubated at room temperature for 10 minutes. The samples were then centrifuged at 12,000×g at 4° C.

RNA Wash

The supernatant was removed and the pellets washed with 1 ml of 75% Ethanol, centrifuged again for 5 minutes at 7,500×g at 4° C.

Redissolving the RNA

The final pellets were briefly air dried and resuspended in nuclease-free, sterile water. An aliquot of the RNA was run on an agarose gel to determine the presence of the 18 and 28 S bands. The concentration was determined by measuring the absorbance at 260 nm.

Taqman:

Taqman primers were ordered for the EST with the accession number AA154371 (EST AA154371 (sequence for the EST: SEQ ID NO 21). Primers for the real time Taqman PCR studies were chosen using Primer Express software (Perkin Elmer) and synthesized by Sigma Genosys. The sequences of the forward and reverse primers were GACAGGGCTGAGGATTCGG (SEQ ID NO: 22) and AGGTCCATGACCACATCTCACA (SEQ ID NO: 23) respectively. PCR product was run on the 4% agarose gel to confirm presence of a single band. PCR reactions were run on ABI Prizm System 7700 sequence detector (Perkin-Elmer) using CybrGreen PCR Core Reagents Kit (Perkin-Elmer) according to the manufacturer's protocol. The optimum final primer concentration in reactions was found to be 0.2 uM. The results were normalized to 18S and expressed as logarithm base 2 of copy number difference with18S RNA levels. Samples from at least 3 independent RT reactions per point were used.

Clinical Assessment of EAE Mice is Described in FIG. 18.

Results

Brain Samples

The mRNA levels of the HMR 1031 and IVL 984 treated EAE mice are statistically significant lower in comparison to the vehicle control mice.

Spleen Samples

The spleen samples do not show the same tendencies as the brain in either treatment (p-value: 0.01-0.05)

The able below is a brief synopsis of the results

| | |
|---|---|
| HMR 1031 | IVL 984 |

| Analyzed gene: | Brain | Spleen | Brain | Spleen |
|---|---|---|---|---|
| EST AA 154371 | tendency | — | ✓ | ✓ |

✓: Statistically significant decrease in macrophage mannose receptor mRNA or protein levels with a 5% or lower probability rate in treated mice compared to vehicle control mice.
tendency: The decrease in mRNA levels is statisically significant a probability rate of 5-15%.
—: The changes in mRNA levels are statisically significant at a probability rate of higher than 15% (not significant).

These results show that levels of the genetic marker EST AA154371 are statistically significantly lower in bodily samples taken from EAE mice treated with a VLA-4 receptor antagonist than levels measured in EAE mice not treated with such an antagonist.

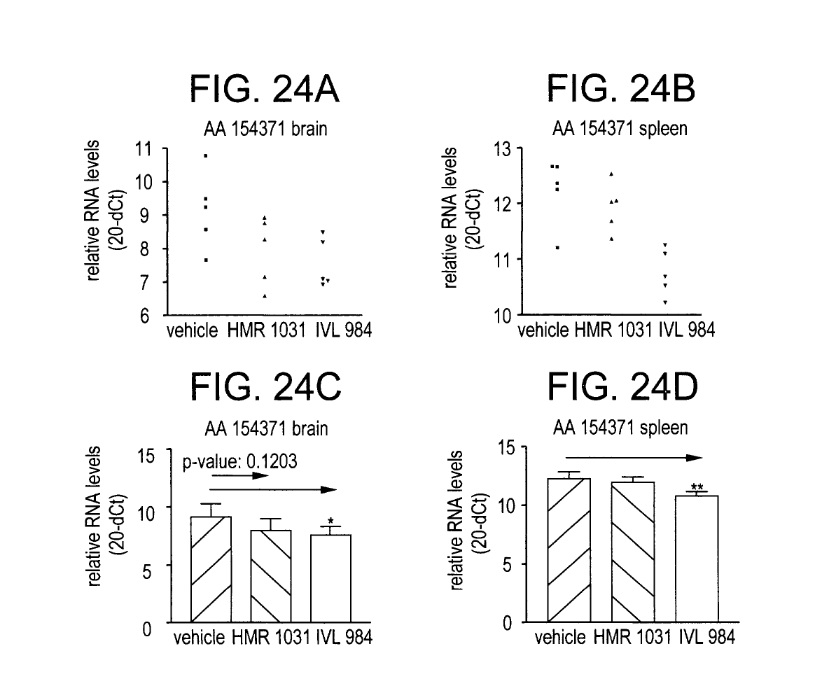

Detailed Taqman Results: FIG. 24:

### Discussion

The results set forth above, as well as in FIG. 24, clearly demonstrate that genetic markers discovered to have modulated expression in a knockout mouse of Example I are surrogate genetic markers for the modulation of the signaling activity of VLA-4 receptor. Thus, they can readily be used to determine whether a compound or agent has efficacy in modulating the signaling activity of VLA-4 receptor. In particular, the genetic marker EST AA154371, which was determined to have decreased levels of expression in an alpha-4 integrin knockout mice of Example I, also has decreased levels of expression in an organism to which a known VLA-4 receptor antagonist, i.e. IVL 984 or HMR1031, is administered. Hence, methods of the present invention can readily be used to identify antagonists of the signaling activity of VLA-4 receptor, which readily have application in treating a plethora of diseases, including, but certainly not limited to asthma, arthritis, and multiple sclerosis, to name only a few. Moreover, methods of the present invention for determining whether a compound or agent has efficacy in modulating signaling activity of VLA-4 receptor can also readily be used to monitor a patient to whom a VLA-4 antagonist is administered, particularly in a clinical setting.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values set forth in the instant specification and claims are approximate, and are provided for description.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values set forth in the instant specification and claims are approximate, and are provided for description.

Various publications are cited herein, the disclosures of which are incorporated by reference in their entireties.

Allen, A. R., McHalle, J., Smith, J., Cook, H. T., Karkar, A., Haskard, D. O., Lobb, R. R., Pusey, C. D., 1999. Endothelial expression of VCAM-1 in experimental crescentic nephritis and effect of antibodies on very late antigen-4 or VCAM-1 on glomerular injury. Journal of Immunology 162 (9):5519-27

Bochner, B. S., Luscinskas, F. W., Gimbrone, M. A. Jr., Newman, W., Sterbinsky, S. A., Derse-Anthony, C. P., Klunk, D., Schleimer, R. P. 1991 Adhesion of human basophils, eosinophils, and neutrophils to interleukin 1-activated human vascular endothelial cells: contributions of endothelial cell adhesion molecules. Journal of Experimental Medicine 173(6):1553-7.

Capecchi, M. R., 1989. Altering the Genome by Homologous Recombination. Science 244:1288-92.

Capecchi, M. R., 1994. Targeted Gene Replacement. Scientific American March 34-41.

Chan, B. M. C., Wong, J. G. P., Rao, A., Hemler, M. E. 1991 T Cell Receptor-Independent, Antigen-Specific Stimulation Of A Murine T Cell Clone Induces A Transient, VLA Protein-Mediated Binding To Extracellular Matrix. The Journal of Immunology 147: 398-404

Chen, L. L., Whitty, A., Lobb, R. R., Adamns, S. P., Pepinsky, R. B. 1999 Multiple Activation States of Integrin α4 β1 Detected through Their Different Affinities for a Small Molecule Ligand. The Journal of Biological Chemistry 274 (19): 13167-13175.

Cote, R. J., Morrisay, D. M., Houghton, A. M., Beatty, E. J. Jr., Oettgen, H. F and Old, L. J., 1983. Generation of human monoclonal antibodies reactive with cellular antigens. Proc. Natl. Acad. Sci. USA 80(7):2026-30.

DeMeirsman, C. Schollen, E., Japers, M., Ongena, K., Matthijs, G., Marynen, P., Cassiman, J. -J. 1994. Cloning and Characterization of the Promoter Region of the Murine Alpha-4 Integrin Subunit. DNA and Cell Biology 13: 743-754

DeMeirsman, C., Jaspers, M., Schollen, E., Cassiman, J. -J. 1996 The Genomic Structure of the Murine alpha-4 Integrin Gene. DNA and Cell Biology 15:595-603

Dunon, D., Piali, L., Imhof, B. A., 1996. To stick or not to stick: the new leukocyte homing paradigm. Current Opinion in Cell Biology 8:714-23.

Fässler, R. and Meyer, M. 1995. Consequences of lack of β1 integrin gene in embryonic stem cells affects morphology, adhesion, and migration but not integration into the inner cell mass of blastocysts. Journal of Cell Biology 128:979-88.

George, E. L. and Hynes, R. O. 1994. Gene Targeting and Generation of Mutant Mica for Studies of Cell-Extracellular Matrix Interactions. Methods in Enzymology 245:368-420

Georges-Labouesse, E., Messaddeq, N., Yehia, G., Cadalbert, L. Dierich, A., LeMeur, M. 1996. Absence of the alpha-6 integrin leads to epidermolysis bullosa and neonatal death in mice. Nature Genetics 13:370-73.

Girard, J. -P. and Springer, T. A. 1995 High endothelial venules (HEVs): specialized endothelium for lymphocyte migration. Immunology Today 16(9):449-56.

Gossen, M. and Bujard, H., 1992. Tight control of gene expression in mammalian cells in tetracycline-responsive promoters. Proc Natl Acad Sci USA 89(12):5547-51.

Hynes, R. O. 1992 Integrins: versatility, modulation, and signaling in cell adhesion. Cell 69: 11-25.

Hynes, R. O. 1994. Genetic analysis of cell-matrix interactions in development. Current Opinion in Genetics and Development 4:569-574

Hynes, R. O. 1996. Targeted Mutations in Cell Adhesion Genes: What Have We Learned from Them? Developmental Biology 180:402-12

Hynes, R. O. and Wagner, D. 1996, Genetic Manipulation of Vascular Adhesion Molecules in Mice. Journal of Clinical Investigation 98(10):2193-2195.

Issekutz, A. C. 1998 Adhesion molecules mediating neutrophil migration to arthritis in vivo and across endothelium and connective tissue barriers in vitro. Inflammation Research 47 Suppl 3:S123-32.

Jakubowski, A., Ehrenfels, B. N., Pepinsly, R. B., Burkly, L. C., 1995. Vascular cell adhesion molecule-Ig fusion protein selectively targets activated α4-integrin receptors in vivo. Journal of Immunology 155:938-46.

Jaspers, M., DeMeirsman, C., Schollen, E., Vekemans, S., Cassiman, J. J., 1994. Stable expression of VLA-4 and increased maturation of the beta 1 -integrin precursor after transfection of CHO cells with alpha 4 m cDNA. FEBS Lett 353(3):239-42.

Jaspers, M., Wu, R. R., Van der Schueren, B., Cassiman, J. J. 1995 Localization of α₄ₘ integrin at sites of mesenchyme condensation during embryonic mouse development. Differentiation 59: 79-86

Kohler, G. and Milstein, C., 1997. Continuous cultures of fused cells secreting antibody of predefined specificity. Nature 256(5517):594-7.

Ley, K. 1995. Gene-Targeted Mice in Leukocyte Adhesion Research. Microcirculation 2(2):141-50

McMurray, R. 1996. Adhesion Molecules in Autoimmune Disease. Seminars in Arthritis and Rheumatism 25(4):215-23.

Mousa, S. A., Cheresh, D. A., 1997. Recent advances in cell adhesion molecules and extracellular matrix proteins: potential clinical implications. DDT 2(5):187-99

Nebert, D. W, Duffy, J. J. 1997. How Knockout Mouse Lines Will Be Used to Study the Role of Drug-Metabolizing Enzymes and Their Receptors during Reproduction and Development, and in Environmental Toxicity, Cancer, and Oxidative Stress. Biochemical Pharmacology 53:249-254.

Neuhaus, H., Hu, M. C. -T., Hemler, M. E., Takada, Y., Holzman, B., Weissman, I. L. 1991, Cloning and Expression of cDNAs for the alpha Subunit of the murine Lymphocyte-Peyer's Patch Adhesion Molecule. The Journal of Cell Biology 115:1149-1158

Newham, P. and Humphries, M. J. 1996 Integrin adhesion receptors: structure, function and implications for biomedicine Molecular Medicine Today 2(7):304-13.

Rosen, G. D., Sanes, J., R., LaChance, R., Cunningham, J. M., Roman, J., Dean, D. C. 1992 Roles for the Integrin VLA-4 and Its Counter Receptor VCAM-1 in Myogenesis. Cell 69: 1107-1119.

Sagara, H., Matsuda, H., Wada, N., Yagita, H., Fukada, T., Okumura, K., Makino, S., Ra, C., 1997. A Monoclonal Antibody against Very Late Activation Antigen-4 Inhibits Eosinophil Accumulation and Late Asthmatic Response in a Guinea Pig Model of Asthma. International Archives of Allergy and Immunology 112:287-94.

Sharpe, A. H. 1995 analysis of lymphocyte costimulation in vivo using transgenic and ‘knockout’ mice. Current Opinion in Immunology 7:389-95.

Shimizu, Y., Van seventer, G. A., Horgan, K. J., Shaw, S. 1990 Regulated expression and binding at three VLA (beta 1) integrin receptors on T cells. Nature 345:250-253.

Stephens, L. E., Sutherland, A. E., Klimanskaya, I. V., Andrieux, A., Meneses, J., Pedersen, R. A., Damsky, C. H., 1995 Detection of β1 integrins in mice results in inner cell mass failure and peri-implantation lethality. Genes Development 9:1883-95.

Taooka, Y., Chen, J., Yednock, T., Sheppard, D. 1999. The integrin alpha9beta1 mediates adhesion to activated endothelial cells and transendothelial neutrophil migration through interaction with vascular cell adhesion molecule-1. Journal of Cell BiologyI 145(2):413-420.

Teratocarcinomas and embryonic stem cells—apractical approach. Edited by E. J. Robertson, IRL Press, 1987.

Van der Neut, R., Krinpenfort, P., Calafat, J. Niessen, C. M., Sonnenberg, P. A., 1996. Epithelial detachment due to absence of hemidesmosomes in beta4 null mice. Nature Genetics 13(3):366-369.

Yang, J. T., Rayburn, H., Hynes, R. O. 1993. Embryonic mesodermal defects in α5 integrin-deficient mice. Development 119:1093-1105.

Yednock, T. A., Cannon, C., Vandevert, C., Goldbach, E. G., Shaw, G., Ellis, D. K., Liaw, C., Fritz, L., Tanner, L. I. 1995 α4 β1 Integrin-dependent Cell Adhesion Is Regulated by a Low Affinity Receptor Pool That Is Conformationally Responsive to Ligand The Journal of Biological Chemistry 270 (48): 28740-28750.

What is claimed is:

1. A mouse that is unable express functional alpha-4 integrin protein.

2. The mouse of claim 1, having a phenotype comprising:
a) no detectable level of a first genetic marker comprising functional alpha-4 integrin; and
b) a modulation of the level of a second genetic marker in the knockout mouse relative to the level of said genetic marker in a control wild type mouse.

3. The mouse of claim 2, wherein said second genetic marker comprises:
Mus musculus anti-von Willebrand factor antibody NMC-4 kappa chain mRNA;
Mouse gene for immunoglobulin alpha heavy chain, switch region and con;
(H-2 class I histocompatibility antigen, d-k alpha chain precursor;
Mus musculus MHC class I Qa-1a antigen mRNA, complete cds;
Mus musculus ribosomal protein L41 mRNA, complete cds;
Mouse MHC class I D-region cell surface antigen (D2d) gene, complete cds;
Mus musculus mRNA for erythroid differentiation regulator, partial;
NRNT(1e-92): , complete sequence [Mus musculus];
vc50e11.r1 Knowles Solter mouse 2 cell Mus musculus cDNA clone 778028;
mt23g11.r1 Soares mouse 3NbMS Mus musculus cDNA clone 621956 5′ TIGR cds;
NRNT(0.0): Mus musculus mRNA for IIGP protein;
Mouse DNA for Ig gamma-chain, secrete-type and membrane-bound, partial;
NRNT(2e-61): Mus musculus DNA for PSMB5, complete cds;
Homologous to sp P32507: poliovirus receptor homolog precursor;
Mouse Ig rearranged H-chain mRNA constant region;
M.musculus mRNA RHAMM;
R74638 MDB0793 Mouse brain, Stratagene Mus musculus cDNA 3′end;
Mus musculus pale ear (ep mutant allele) mRNA, partial cds;
mj35h09.r1 Soares mouse embryo NbME13.5 14.5 Mus musculus cDNA clone 4;
MUSGS00761 Mouse 3′-directed cDNA; MUSGS00761; clone mb1494. TIGR clus;
Homologous to sp P41725: brain enriched hyaluronan binding protein PRE;
M.musculus mRNA for D2A dopamine receptor;
mo54b0.5.r1 Life Tech mouse embryo 10 5 dpc 10665016 Mus musculus cDNA cds;
vm06f11.r1 Knowles Solter mouse blastocyst B1 Mus musculus cDNA clone;
Mus musculus Major Histocompatibility Locus class II region;
Mus musculus capping protein beta-subunit isoform 1 mRNA, complete cds;
Mus musculus mRNA for peroxisomal integral membrane protein PMP34;
Mus musculus mRNA for JAB, complete cds;
Mouse interferon regulatory factor 1 mRNA, complete cds;
Mus musculus GTPase IGTP mRNA, complete cds;
Mouse spi2 proteinase inhibitor (spi2/eb1) mRNA, 3 end;
Homologous to sp Q01514: Interferon-Induced Guanylate-Binding Protein;
Homologous to sp P13765: HLA CLASS II histocompatibility antigen, DO B;
NRNT(3e-39): Human phosphatidylinositol (4,5)bisphosphate 5-phosphatase;
Mus musculus (clone U2) T-cell specific protein mRNA, complete cds;
Mus musculus mRNA for peroxisomal integral membrane protein PMP34;
M. musculus mRNA for macrophage mannose receptor; or
the concentration of progenitor stem cells in blood.

4. The mouse of claim 3, wherein the modulation of the level of the said second genetic marker comprises an increase in the level of said second genetic marker measured in said mouse relative to the level of said second genetic marker measured in said control wild type mouse, wherein said second genetic marker comprises:
Mus musculus anti-von Willebrand factor antibody NMC-4 kappa chain mRNA;
Mouse gene for immunoglobulin alpha heavy chain, switch region and con;
(H-2 CLASS I histocompatibility antigen, D-K alpha chain precursor;
Mus musculus MHC class I Qa-1a antigen mRNA, complete cds;
Mus musculus ribosomal protein L41 mRNA, complete cds;
Mouse MHC class I D-region cell surface antigen (D2d) gene, complete c;
Mus musculus mRNA for erythroid differentiation regulator, partial;
NRNT(1e-92):, complete sequence [Mus musculus];
vc50e11.r1 Knowles Solter mouse 2 cell Mus musculus cDNA clone 778028;
NRNT(0.0): Mus musculus mRNA for IIGP protein;
Mouse DNA for Ig gamma-chain, secrete-type and membrane-bound, partial;
NRNT(2e-61): Mus musculus DNA for PSMB5, complete cds;
Homologous to sp P32507: Poliovirus Receptor Homolog Precursor;
Mouse Ig rearranged H-chain mRNA constant region;
M.musculus mRNA RHAMM;
R74638 MDB0793 Mouse brain, Stratagene Mus musculus cDNA 3′end;
Mus musculus pale ear (ep mutant allele) mRNA, partial cds;
mj35h09.r1 Soares mouse embryo NbME13.5 14.5 Mus musculus cDNA clone 4;
MUSGS00761 Mouse 3′-directed cDNA; MUSGS00761; clone mb1494. TIGR clus;
Homologous to sp P41725: brain enriched hyaluronan binding protein PRE;
M.musculus mRNA for D2A dopamine receptor;
mo54b05.r1 Life Tech mouse embryo 10 5 dpc 10665016 Mus musculus cDNA cds;
mt23g11.r1 Soares mouse 3NbMS Mus musculus cDNA clone 621956 5′ TIGR c;
Mus musculus Bop1 mRNA, complete cds;
C75959 Mouse 3.5-dpc blastocyst cDNA Mus musculus cDNA clone J0001C05; or
the concentration of progenitor stem cells in blood.

5. The knockout mouse of claim 3, wherein the modulation of the level of said second genetic marker comprises a decrease in the level of said second genetic marker measured in said knockout mouse relative to the level of said second genetic marker measured in said control wild type mouse, wherein said second genetic marker comprises:
vm06f11.r1 Knowles Solter mouse blastocyst B1 Mus musculus cDNA clone;
Mus musculus Major Histocompatibility Locus class II region;
Mus musculus capping protein beta-subunit isoform 1 mRNA, complete cds;
Mus musculus mRNA for peroxisomal integral membrane protein PMP34;
Mus musculus mRNA for JAB, complete cds;
Mouse interferon regulatory factor 1 mRNA, complete cds;
Mus musculus GTPase IGTP mRNA, complete cds;
Mouse spi2 proteinase inhibitor (spi2/eb1) mRNA, 3 end;
Homologous to sp Q01514: Interferon-Induced Guanylate-Binding Protein;
Homologous to sp P13765: HLA Class II histocompatibility antigen, DO B;
NRNT(3e-39): Human phosphatidylinositol (4,5)bisphosphate 5-phosphastase;
Mus musculus (clone U2) T-cell specific protein mRNA, complete cds; or
M. musculus mRNA for macrophage mannose receptor.

6. The mouse of claim 1, wherein said mouse is a knockout mouse whose genome has a first and second allele capable of expressing functional alpha-4 integrin protein, wherein:
(a) said first allele comprises a defect that prevents said first allele from expressing functional alpha-4 integrin protein;
(b) said second allele comprises a defect that prevents said second allele from expressing functional alpha-4 integrin protein; and
(c) said genome comprises two copies a transgene comprising a portion of a cDNA molecule that encodes alpha-4 integrin promoter operatively associated with a promoter,
wherein said knockout mouse is unable to express functional alpha-4 integrin protein.

7. The knockout mouse of claim 6, wherein said defect comprises a substitution, insertion, and/or deletion of one or more nucleotides in said first allele and in said second allele.

8. The knockout mouse of claim 6, wherein said transgene comprises a portion of said isolated cDNA molecule that encodes for alpha-4 integrin protein operatively associated with a tetP promoter, and comprises a DNA sequence of SEQ ID NO: 1.

9. A knockout mouse that is unable to express functional alpha-4 integrin protein, wherein said knockout mouse has a genome comprising:
(a) first and second alleles capable of expressing functional alpha-4 integrin protein that have defects that prevent the alleles from expressing functional alpha-4 integrin protein; and
(b) two copies of a transgene comprising a DNA sequence of SEQ ID NO: 1, wherein said knockout mouse is unable to express functional alpha-4 integrin protein.

10. A method for making a knockout mouse that is unable to express functional alpha-4 integrin protein, comprising the steps of:
(a) crossing two knockout mice comprising a first and second allele capable of expressing functional alpha-4 integrin protein, wherein the knockout mice each comprise a defect in either the first allele or second allele, such that either the first or second allele in each knockout mouse is unable to express functional alpha-4 integrin protein;
(b) harvesting embryos resulting from the cross of step (a), wherein the embryos are heterozygous for the defect;
(c) inserting a transgene comprising a portion of an isolated cDNA molecule that encodes for alpha-4 integrin protein operatively associated with a promoter, into each embryo harvested in step (b), to form a transfected embryo;
(d) inserting the transfected embryo into a pseudopregnant female mouse so that the pseudopregnant female mouse gives birth to a mouse whose genome comprises:
(i) first and second alleles capable of expressing functional alpha-4 integrin protein, wherein either the first or the second allele comprises the defect that prevents the allele from expressing functional alpha-4 integrin protein, and
(ii) the transgene integrated into said genome; and
(e) crossing two mice produced in step (d) to produce an alpha-4 homozygous knockout mouse whose genome comprises two copies of the transgene,
wherein the resulting knockout mouse of step (e) is unable to express functional alpha-4 integrin protein.

11. The method of claim 10 for making a knockout mouse that is unable to express functional alpha-4 integrin protein, wherein the defect in either the first allele or second allele in step (a) comprises a disruption of the first allele or the second allele, such that the first allele or the second allele are unable to express functional alpha-4 integrin protein.

12. The method of claim 10 for making a knockout mouse that is unable to express functional alpha-4 integrin protein, wherein the transgene comprises a DNA sequence of SEQ ID NO: 1.

13. The method of claim 10 for making a knockout mouse that is unable to express functional alpha-4 integrin protein, wherein the step of inserting the transgene into the embryo comprises:
(a) inserting the transgene into an expression vector; and
(b) inserting the vector into the embryo.

14. The method of claim 10 for making a knockout mouse that is unable to express functional alpha-4 integrin protein, wherein the two heterozygous alpha-4 knockout mice of step (a) are assigned Jackson Laboratories stock number 002463.

15. A method for making a knockout mouse that is unable to express functional alpha-4 integrin protein, comprising the steps of:
(a) crossing two heterozygous alpha-4 integrin knockout mice assigned Jackson laboratories stock number 002463;
(b) harvesting the embryos that result from the cross of step (a);
(c) inserting a transgene-comprising a DNA sequence of SEQ ID NO: 1 into each embryo harvested in step (b), to form a transfected embryo;
(d) inserting the transfected embryo into a pseudopregnant female mouse so that the pseudopregnant female mouse gives birth to an alpha-4 integrin heterozygous knockout mouse having the transgene within its genome; and
(e) crossing two knockout mice produced in step (d) to produce a homozygous alpha-4 integrin knockout mouse whose genome comprises two copies of the transgene;
so that the knockout mouse of step (e) is unable to express functional alpha-4 integrin protein.

16. A method for assaying an agent for potential activity as an alpha-4 integrin protein antagonist, comprising the steps of:
(a) administering the agent to a wild type mouse;
(b) measuring the level of a genetic marker in the wild type mouse; and
(c) comparing the measurement of step (b) with the level of the genetic marker measured in a control wild type mouse,
wherein modulation of the level of the genetic marker measured in the wild type mouse relative to the level of the genetic marker measured in the control wild type mouse indicates the agent may possess alpha-4 integrin protein antagonist activity.

17. The method of claim 16, wherein the genetic marker comprises:
Mus musculus anti-von Willebrand factor antibody NMC-4 kappa chain mRNA;
Mouse gene for immunoglobulin alpha heavy chain, switch region and con;
(H-2 class I histocompatibility antigen, d-k alpha chain precursor;
Mus musculus MHC class I Qa-1a antigen mRNA, complete cds;
Mus musculus ribosomal protein L41 mRNA, complete cds;
Mouse MHC class I D-region cell surface antigen (D2d) gene, complete cds;
Mus musculus mRNA for erythroid differentiation regulator, partial;
NRNT(1e-92): , complete sequence [Mus musculus];
vc50e11.r1 Knowles Solter mouse 2 cell Mus musculus cDNA clone 778028;
mt23g11.r1 Soares mouse 3NbMS Mus musculus cDNA clone 621956 5′ TIGR cds;
NRNT(0.0): Mus musculus mRNA for IIGP protein;
Mouse DNA for Ig gamma-chain, secrete-type and membrane-bound, partial;
NRNT(2e-61): Mus musculus DNA for PSMB5, complete cds;
Homologous to sp P32507: poliovirus receptor homolog precursor;
Mouse Ig rearranged H-chain mRNA constant region;
M.musculus mRNA RHAMM;
R74638 MDB0793 Mouse brain, Stratagene Mus musculus cDNA 3′end;
Mus musculus pale ear (ep mutant allele) mRNA, partial cds;
mj35h09.r1 Soares mouse embryo NbME13.5 14.5 Mus musculus cDNA clone 4;
MUSGS00761 Mouse 3′-directed cDNA; MUSGS00761; clone mb1494. TIGR clus;
Homologous to sp P41725: brain enriched hyaluronan binding protein PRE;
M.musculus mRNA for D2A dopamine receptor;
mo54b05.r1 Life Tech mouse embryo 10 5 dpc 10665016 Mus musculus cDNA cds;
Mus musculus Bop1 mRNA, complete cds;
C75959 Mouse 3.5-dpc blastocyst cDNA Mus musculus cDNA clone J0001C05;
vm06f11.r1 Knowles Solter mouse blastocyst B1 Mus musculus cDNA clone;
Mus musculus Major Histocompatibility Locus class II region;
Mus musculus capping protein beta-subunit isoform 1 mRNA, complete cds;
Mus musculus mRNA for peroxisomal integral membrane protein PMP34;
Mus musculus mRNA for JAB, complete cds;
Mouse interferon regulatory factor 1 mRNA, complete cds;
Mus musculus GTPase IGTP mRNA, complete cds;
Mouse spi2 proteinase inhibitor (spi2/eb1) mRNA, 3 end;
Homologous to sp Q01514: Interferon-Induced Guanylate-Binding Protein;
Homologous to sp P13765: HLA CLASS II histocompatibility antigen, DO B;
NRNT(3e-39): Human phosphatidylinositol (4,5)bisphosphate 5-phosphatase;
Mus musculus (clone U2) T-cell specific protein mRNA, complete cds;
Mus musculus mRNA for peroxisomal integral membrane protein PMP34;
M. musculus mRNA for macrophage mannose receptor; or
the concentration of progenitor stem cells in blood.

18. The method of claim 17, wherein the modulation of the level of the genetic marker measured in the wild type mouse comprises an increase relative to the level of the genetic marker measured in the control wild type mouse, wherein the genetic marker comprises:
Mus musculus anti-von Willebrand factor antibody NMC-4 kappa chain mRNA;
Mouse gene for immunoglobulin alpha heavy chain, switch region and con;
(H-2 CLASS I histocompatibility antigen, D-K alpha chain precursor;
Mus musculus MHC class I Qa-1a antigen mRNA, complete cds;
Mus musculus ribosomal protein L41 mRNA, complete cds;
Mouse MHC class I D-region cell surface antigen (D2d) gene, complete c;
Mus musculus mRNA for erythroid differentiation regulator, partial;
NRNT(1e-92):, complete sequence [Mus musculus];
vc50e11.r1 Knowles Solter mouse 2 cell Mus musculus cDNA clone 778028;
NRNT(0.0): Mus musculus mRNA for IIGP protein;
Mouse DNA for Ig gamma-chain, secrete-type and membrane-bound, partial;
NRNT(2e-61): Mus musculus DNA for PSMB5, complete cds;
Homologous to sp P32507: Poliovirus Receptor Homolog Precursor;
Mouse Ig rearranged H-chain mRNA constant region;
M.musculus mRNA RHAMM;
R74638 MDB0793 Mouse brain, Stratagene Mus musculus cDNA 3′end;
Mus musculus pale ear (ep mutant allele) mRNA, partial cds;
mj35h09.r1 Soares mouse embryo NbME13.5 14.5 Mus musculus cDNA clone 4;
MUSGS00761 Mouse 3′-directed cDNA; MUSGS00761; clone mb1494, TIGR clus;
Homologous to sp P41725: brain enriched hyaluronan binding protein PRE;
M.musculus mRNA for D2A dopamine receptor;
mo54b05.r1 Life Tech mouse embryo 10 5 dpc 10665016 Mus musculus cDNA cds;
mt23g11.r1 Soares mouse 3NbMS Mus musculus cDNA clone 621956 5′ TIGR c;
Mus musculus Bop1 mRNA, complete cds;
C75959 Mouse 3.5-dpc blastocyst cDNA Mus musculus cDNA clone J0001C05; or
the concentration of progenitor stem cells in blood.

19. The method of claim 17, wherein modulation of the level of the genetic marker comprises a decrease in the level of the genetic marker measured in the wild type mouse relative to the level of the genetic marker measured in the control wild type mouse, wherein the genetic marker comprises:
vm06f11.r1 Knowles Solter mouse blastocyst B1 Mus musculus cDNA clone;
Mus musculus Major Histocompatibility Locus class II region;
Mus musculus capping protein beta-subunit isoform 1 mRNA, complete cds;
Mus musculus mRNA for peroxisomal integral membrane protein PMP34;
Mus musculus mRNA for JAB, complete cds;
Mouse interferon regulatory factor 1 mRNA, complete cds;
Mus musculus GTPase IGTP mRNA, complete cds;
Mouse spi2 proteinase inhibitor (spi2/eb1) mRNA, 3 end;
Homologous to sp Q01514: Interferon-Induced Guanylate-Binding Protein;
Homologous to sp P13765: HLA Class II histocompatibility antigen, DO B;
NRNT(3e-39): Human phosphatidylinositol (4,5)bisphosphate 5-phosphatase;
Mus musculus (clone U2) T-cell specific protein mRNA, complete cds; or
M. musculus mRNA for macrophage mannose receptor.

20. A method for assaying an agent for activity in ameliorating deleterious side effects associated with an alpha-4 integrin protein antagonist, comprising the steps of:
(a) administering the agent to a mouse that is unable to express functional alpha-4 integrin;
(b) measuring the level of a genetic marker in the mouse; and
(c) comparing the level of the genetic marker measured in the mouse to the level of the genetic marker measured in a control mouse that is unable to express functional alpha 4 integrin,
wherein a modulation of the level of the genetic marker measured in the mouse relative to the level of the genetic marker measured in the control mouse indicates the agent may have activity in ameliorating deleterious side effects associated with an alpha-4 integrin protein antagonist.

21. The method of claim 20, wherein the genetic marker comprises:
Mus musculus anti-von Willebrand factor antibody NMC-4 kappa chain mRNA;
Mouse gene for immunoglobulin alpha heavy chain, switch region and con;
(H-2 class I histocompatibility antigen, d-k alpha chain precursor;
Mus musculus MHC class I Qa-1a antigen mRNA, complete cds;
Mus musculus ribosomal protein L41 mRNA, complete cds;
Mouse MHC class I D-region cell surface antigen (D2d) gene, complete cds;
Mus musculus mRNA for erythroid differentiation regulator, partial;
NRNT(1e-92): , complete sequence [Mus musculus];
vc50e11.r1 Knowles Solter mouse 2 cell Mus musculus cDNA clone 778028;
mt23g11.r1 Soares mouse 3NbMS Mus musculus cDNA clone 621956 5′ TIGR cds;
NRNT(0.0): Mus musculus mRNA for IIGP protein;
Mouse DNA for Ig gamma-chain, secrete-type and membrane-bound, partial;
NRNT(2e-61): Mus musculus DNA for PSMB5, complete cds;
Homologous to sp P32507: poliovirus receptor homolog precursor;
Mouse Ig rearranged H-chain mRNA constant region;
M.musculus mRNA RHAMM;
R74638 MDB0793 Mouse brain, Stratagene Mus musculus cDNA 3′end;
Mus musculus pale ear (ep mutant allele) mRNA, partial cds;
mj35h09.r1 Soares mouse embryo NbME13.5 14.5 Mus musculus cDNA clone 4;
MUSGS00761 Mouse 3′-directed cDNA; MUSGS00761; clone mb1494. TIGR clus;
Homologous to sp P41725: brain enriched hyaluronan binding protein PRE;
M.musculus mRNA for D2A dopamine receptor;
mo54b05.r1 Life Tech mouse embryo 10 5 dpc 10665016 Mus musculus cDNA cds;
Mus musculus Bop1 mRNA, complete cds;
C75959 Mouse 3.5-dpc blastocyst cDNA Mus musculus cDNA clone J0001C05;
vm06f11.r1 Knowles Solter mouse blastocyst B1 Mus musculus cDNA clone;
Mus musculus Major Histocompatibility Locus class II region;
Mus musculus capping protein beta-subunit isofomi 1 mRNA, complete cds;
Mus musculus mRNA for peroxisomal integral membrane protein PMP34;
Mus musculus mRNA for JAB, complete cds;
Mouse interferon regulatory factor 1 mRNA, complete cds;
Mus musculus GTPase IGTP mRNA, complete cds;
Mouse spi2 proteinase inhibitor (spi2/eb1) mRNA, 3 end;
Homologous to sp Q01514: Interferon-Induced Guanylate-Binding Protein;
Homologous to sp P13765: HLA CLASS II histocompatibility antigen, DO B;
NRNT(3e-39): Human phosphatidylinositol (4,5)bisphosphate 5-phosphatase;
Mus musculus (clone U2) T-cell specific protein mRNA, complete cds;
Mus musculus mRNA for peroxisomal integral membrane protein PMP34;
M. musculus mRNA for macrophage mannose receptor; or
the concentration of progenitor stem cells in blood.

22. The method of claim 21, wherein the modulation is an increase in the level of the genetic marker measured in the mouse relative to level of the genetic marker measured in the control mouse, wherein the genetic marker comprises:
Mus musculus anti-von Willebrand factor antibody NMC-4 kappa chain mRNA;
Mouse gene for immunoglobulin alpha heavy chain, switch region and con;
(H-2 CLASS I histocompatibility antigen, D-K alpha chain precursor;
Mus musculus MHC class I Qa-1a antigen mRNA, complete cds;
Mus musculus ribosomal protein L41 mRNA, complete cds;
Mouse MHC class I D-region cell surface antigen (D2d) gene, complete c;
Mus musculus mRNA for exythroid differentiation regulator, partial;
NRNT(1e-92): , complete sequence [Mus musculus];
vc50e11.r1 Knowles Solter mouse 2 cell Mus musculus cDNA clone 778028;
NRNT(0.0): Mus musculus mRNA for IIGP protein;
Mouse DNA for Ig gamma-chain, secrete-type and membrane-bound, partial;
NRNT(2e-61): Mus musculus DNA for PSMB5, complete cds;
Homologous to sp P32507: Poliovirus Receptor Homolog Precursor;
Mouse Ig rearranged H-chain mRNA constant region;
M.musculus mRNA RHAMM;
R74638 MDB0793 Mouse brain, Stratagene Mus musculus cDNA 3′end;
Mus musculus pale ear (ep mutant allele) mRNA, partial cds;
mj35h09.r1 Soares mouse embryo NbME13.5 14.5 Mus musculus cDNA clone 4;
MUSGS00761 Mouse 3′-directed cDNA; MUSGS00761; clone mb1494. TIGR clus;
Homologous to sp P41725: brain enriched hyaluronan binding protein PRE;
M.musculus mRNA for D2A dopamine receptor;
mo54b05.r1 Life Tech mouse embryo 10 5 dpc 10665016 Mus musculus cDNA cds;
mt23g11.r1 Soares mouse 3NbMS Mus musculus cDNA clone 621956 5′ TIGR c;
Mus musculus Bop1 mRNA, complete cds;
C75959 Mouse 3.5-dpc blastocyst cDNA Mus musculus cDNA clone J0001C05; or
the concentration of progenitor stem cells in blood.

23. The method of claim 21, wherein the modulation is a decrease in the level of the genetic marker measured in the knockout mouse relative to level of the genetic marker measured in the control knockout mouse, wherein the genetic marker comprises:
vm06f11.r1 Knowles Solter mouse blastocyst B1 Mus musculus cDNA clone;
Mus musculus Major Histocompatibility Locus class II region;
Mus musculus capping protein beta-subunit isoform 1 mRNA, complete cds;
Mus musculus mRNA for peroxisomal integral membrane protein PMP34;
Mus musculus mRNA for JAB, complete cds;
Mouse interferon regulatory factor 1 mRNA, complete cds;
Mus musculus GTPase IGTP mRNA, complete cds;
Mouse spi2 proteinase inhibitor (spi2/eb1) mRNA, 3 end;
Homologous to sp Q01514: Interferon-Induced Guanylate-Binding Protein;
Homologous to sp P13765: HLA Class II histocompatibility antigen, DO B;
NRNT(3e-39): Human phosphatidylinositol (4,5)bisphosphate 5-phosphatase;
Mus musculus (clone U2) T-cell specific protein mRNA, complete cds; or
M. musculus mRNA for macrophage mannose receptor.

24. The method of claim 20, wherein the mouse and the control mouse are knockout mice whose genomes comprise:
(a) first and second alleles capable of expressing functional alpha-4 integrin protein that have defects that prevent the alleles from expressing functional alpha-4 integrin protein; and
(b) two copies a transgene comprising a portion of an isolated cDNA molecule that encodes for an alpha-4 integrin protein, operatively associated with a promoter.

25. The method of claim 24, wherein the transgene comprises a portion of a cDNA molecule that encodes alpha-4 integrin protein operatively associated with a tetP promoter, and the transgene comprises a DNA sequence of SEQ ID NO: 1.

26. A method for assaying an agent for potential alpha-4 integrin protein antagonist activity, comprising the steps of:
(a) removing a first blood sample from a mammal and measuring the concentration of progenitor stem cells in the first blood sample;
(b) administering the agent to the mammal;
(c) removing a second blood sample from the mammal and measuring the concentration of progenitor stem cells in the second blood sample; and
(d) comparing the measured concentration of progenitor stem cells in the first blood sample with measured concentration of progenitor stem cells in the second blood sample,
wherein an increase in the measured progenitor stem cell concentration in the second blood sample relative to the measured progenitor stem cell concentration in the first blood sample indicates the agent may have alpha-4 integrin protein antagonist activity.

27. The method of claim 26, wherein the mammal is ovine, bovine, equine, canine, feline, murine, or human.

28. A method for assaying an agent for potential alpha-4 integrin protein antagonist activity, comprising the steps of:
(a) administering the agent to a mammal;
(b) measuring the concentration of progenitor stem cells in the blood of the mammal; and
(c) comparing the measured concentration of progenitor stem cells in the blood of the mammal to the measured concentration or progenitor stem cells in the blood of a control mammal,
wherein an increase in the concentration of progenitor stem cells in the blood of the mammal relative to the concentration of progenitor stem cells in the blood of the control mammal is indicative of potential alpha-4 integrin protein antagonist activity in the agent.

29. The method of claim 28, wherein the mammal is ovine, bovine, equine, canine, feline, murine, or human.

30. The mouse of claim 1, wherein said mouse is a transgenic mouse whose genome:
(a) does not possess an allele capable of expressing functional alpha-4 integrin protein; and
(b) comprises two copies of a transgene that comprises a portion of an isolated cDNA molecule that encodes for a functional alpha-4 integrin functional protein, operatively associated with a promoter.

31. The mouse of claim 30, wherein said transgene comprises portion of said isolated cDNA molecule that encodes for functional alpha-4 integrin protein operatively associated with a tetP promoter, and comprises a DNA sequence of SEQ ID NO: 1.

32. A method for determining whether a compound or agent modulates signaling activity of a VLA-4 receptor, comprising the steps of:
(a) administering the compound or agent to an organism;
(b) measuring the expression level of a genetic marker for VLA-4 receptor signaling in a bodily sample removed from the organism; and
(c) comparing the expression level of the genetic marker of step (b) with the expression level of the genetic marker measured in a control bodily sample,
wherein a difference between the measured expression level of the genetic marker in the bodily sample and the control bodily sample indicates that the compound or agent modulates the signaling of the VLA-4 receptor.

33. The method of claim 32, wherein the control bodily sample comprises a bodily sample taken from the organism prior to administration of the compound or agent, or a bodily sample taken from a substantially similar organism to which the compound or agent was not administered.

34. The method of claim 32, wherein the genetic marker comprises:
Mus musculus anti-von Willebrand factor antibody NMC-4 kappa chain mRNA;
Mouse gene for immunoglobulin alpha heavy chain, switch region and con;
(H-2 class I histocompatibility antigen, d-k alpha chain precursor;
Mus musculus MHC class I Qa-1a antigen mRNA, complete cds;
Mus musculus ribosomal protein L41 mRNA, complete cds;
Mouse MHC class I D-region cell surface antigen (D2d) gene, complete cds;
Mus musculus mRNA for erythroid differentiation regulator, partial;
NRNT(1e-92):, complete sequence [Mus musculus];
vc50e11.r1 Knowles Solter mouse 2 cell Mus musculus cDNA clone 778028;
mt23g11.r1 Soares mouse 3NbMS Mus musculus cDNA clone 621956 5′ TIGR cds;
NRNT(0.0): Mus musculus mRNA for IIGP protein;
Mouse DNA for Ig gamma-chain, secrete-type and membrane-bound, partial;
NRNT(2e-61): Mus musculus DNA for PSMB5, complete cds;
Homologous to sp P32507: poliovirus receptor homolog precursor;
Mouse Ig rearranged H-chain mRNA constant region;
M.musculus mRNA RHAMM;
R74638 MDB0793 Mouse brain, Stratagene Mus musculus cDNA 3′end;
Mus musculus pale ear (ep mutant allele) mRNA, partial cds;
mj35h09.r1 Soares mouse embryo NbME13.5 14.5 Mus musculus cDNA clone 4;
MUSGS00761 Mouse 3′-directed cDNA; MUSGS00761; clone mb1494. TIGR clus;
Homologous to sp P41725: brain enriched hyaluronan binding protein PRE;
M.musculus mRNA for D2A dopamine receptor;
mo54b05.r1 Life Tech mouse embryo 10 5 dpc 10665016 Mus musculus cDNA cds;
Mus musculus Bop1 mRNA, complete cds;
C75959 Mouse 3.5-dpc blastocyst cDNA Mus musculus cDNA clone J0001C05;
vm06f11.r1 Knowles Solter mouse blastocyst B1 Mus musculus cDNA clone;
Mus musculus Major Histocompatibility Locus class II region;
Mus musculus capping protein beta-subunit isoform 1 mRNA, complete cds;
Mus musculus mRNA for peroxisomal integral membrane protein PMP34;
Mus musculus mRNA for JAB, complete cds;
Mouse interferon regulatory factor 1 mRNA, complete cds;
Mus musculus GTPase IGTP mRNA, complete cds;
Mouse spi2 proteinase inhibitor (spi2/eb1) mRNA, 3 end;
Homologous to sp Q01514: Interferon-Induced Guanylate-Binding Protein;
Homologous to sp P13765: HLA CLASS II histocompatibility antigen, DO B;
NRNT(3e-39): Human phosphatidylinositol (4,5)bisphosphate 5-phosphatase;
Mus musculus (clone U2) T-cell specific protein mRNA, complete cds;
Mus musculus mRNA for peroxisomal integral membrane protein PMP34;
M. musculus mRNA for macrophage mannose receptor; or
the concentration of progenitor stem cells in blood.

35. The method of claim 34, wherein the expression level of the genetic marker in the bodily sample is less than the expression level of the genetic marker measured in the control bodily sample, which indicates that the compound or agent antagonizes the signaling activity of the VLA-4 receptor, wherein the genetic marker comprises:
vm06f11.r1 Knowles Solter mouse blastocyst B1 Mus musculus cDNA clone;
Mus musculus Major Histocompatibility Locus class II region;
Mus musculus capping protein beta-subunit isoform 1 mRNA, complete cds;
Mus musculus mRNA for peroxisomal integral membrane protein PMP34;
Mus musculus mRNA for JAB, complete cds;
Mouse interferon regulatory factor 1 mRNA, complete cds;
Mus musculus GTPase IGTP mRNA, complete cds;
Mouse spi2 proteinase inhibitor (spi2/eb1) mRNA, 3 end;
Homologous to sp Q01514: Interferon-Induced Guanylate-Binding Protein;
Homologous to sp P13765: HLA Class II histocompatibility antigen, DO B;
NRNT(3 e-39): Human phosphatidylinositol (4,5)bisphosphate 5-phosphatase;
Mus musculus (clone U2) T-cell specific protein mRNA, complete cds; or
M. musculus mRNA for macrophage mannose receptor.

36. The method of claim 34, wherein the expression level of the genetic marker in the bodily sample is greater than the expression level of the genetic marker measured in the control bodily sample, which indicates that the compound or agent antagonizes the signaling activity of the VLA-4 receptor, wherein the genetic marker comprises:
Mus musculus anti-von Willebrand factor antibody NMC-4 kappa chain mRNA;
Mouse gene for immunoglobulin alpha heavy chain, switch region and con;
(H-2 CLASS I histocompatibility antigen, D-K alpha chain precursor;
Mus musculus MHC class I Qa-1a antigen mRNA, complete cds;
Mus musculus ribosomal protein L41 mRNA, complete cds;
Mouse MHC class I D-region cell surface antigen (D2d) gene, complete c;
Mus musculus mRNA for erythroid differentiation regulator, partial;
NRNT(1e-92): , complete sequence [Mus musculus];
vc50e11.r1 Knowles Solter mouse 2 cell Mus musculus cDNA clone 778028;
NRNT(0.0): Mus musculus mRNA for HGP protein;
Mouse DNA for Ig gamma-chain, secrete-type and membrane-bound, partial;
NRNT(2e-61): Mus musculus DNA for PSMB5, complete cds;
Homologous to sp P32507: Poliovirus Receptor Homolog Precursor;
Mouse Ig rearranged H-chain mRNA constant region;
M.musculus mRNA RHAMM;
R74638 MDB0793 Mouse brain, Stratagene Mus musculus cDNA 3′end;
Mus musculus pale ear (ep mutant allele) mRNA, partial cds;
mj35h09.r1 Soares mouse embryo NbME13.5 14.5 Mus musculus cDNA clone 4;
MUSGS00761 Mouse 3′-directed cDNA; MUSGS00761; clone mb1494. TIGR clus;
Homologous to sp P41725: brain enriched hyaluronan binding protein PRE;
M.musculus mRNA for D2A dopamine receptor;
mo54b05.r1 Life Tech mouse embryo 10 5 dpc 10665016 Mus musculus cDNA cds;
mt23g11.r1 Soares mouse 3NbMS Mus musculus cDNA clone 621956 5′ TIGR c;
Mus musculus Bop1 mRNA, complete cds;
C75959 Mouse 3.5-dpc blastocyst cDNA Mus musculus cDNA clone J0001C05; or
the concentration of progenitor stem cells in blood.

37. The method of claim 32, wherein the expression level of the genetic marker measured in the bodily sample is less than the expression level of the genetic marker measured in the control bodily sample which indicates that the compound or agent antagonizes the signaling activity of the VLA-4 receptor, and the genetic marker is selected from the group consisting of macrophage mannose receptor mRNA., Mus musculus mRNA for JAB, complete cds, EST571535, and EST AA154371.

38. The method of claim 32, wherein the compound or agent comprises a protein, a chemical compound, or a nucleotide sequence, a hormone, a carbohydrate or a lectin.

39. The method of claim 38, wherein the compound or agent is an antibody having the VLA-4 receptor as an immunogen, an antisense molecule that hybridizes to VLA-4 receptor mRNA, or a ribozyme that cleaves VLA-4 receptor mRNA.

40. A method for determining the efficacy of a potential antagonist of the signaling of a VLA-4 receptor, wherein such a method comprises the steps of:
(a) removing a first bodily sample from an organism;
(b) measuring the level of macrophage mannose receptor mRNA genetic marker in the first bodily sample;
(c) administering the potential antagonist to the organism;
(d) removing a second bodily sample from the organism;
(e) measuring the level of the genetic marker macrophage mannose receptor mRNA in the second bodily sample; and
(f) comparing the measured levels of step (b) and step (e),
wherein a decrease in the level of the genetic marker measured in step (e) as compared to the level of the genetic marker measured in step (b) indicates the potential antagonist has efficacy in antagonizing the signaling activity of VLA-4.

41. The method of claim 40, wherein the first and second bodily samples comprise a bodily fluid, a bodily tissue, or a combination thereof.

42. The method of claim 40, wherein the potential antagonist comprises a protein, a nucleotide sequence, a chemical compound, or a combination thereof.

43. The method of claim 34, wherein the organism is a mammal.

44. A method for determining the ability of a compound or agent to modulate, and particularly to antagonize, the signaling activity of VLA-4 receptor, comprising the steps of:
(a) contacting the compound or agent with a bodily sample from an organism;
(b) measuring the expression level of a genetic marker for VLA-4 receptor signaling in the bodily sample; and
(c) comparing the expression level of the genetic marker measured in step (b) with the expression level of the genetic marker measured in a control bodily sample.

45. The method of claim 44, wherein the genetic marker comprises:
Mus musculus anti-von Willebrand factor antibody NMC-4 kappa chain mRNA;
Mouse gene for immunoglobulin alpha heavy chain, switch region and con;
(H-2 class I histocompatibility antigen, d-k alpha chain precursor;
Mus musculus MHC class I Qa-1a antigen mRNA, complete cds;
Mus musculus ribosomal protein L41 mRNA, complete cds;
Mouse MHC class I D-region cell surface antigen (D2d) gene, complete cds;
Mus musculus mRNA for erythroid differentiation regulator, partial;
NRNT(1e-92):, complete sequence [Mus musculus];
vc50e11.r1 Knowles Solter mouse 2 cell Mus musculus cDNA clone 778028;
mt23g11.r1 Soares mouse 3NbMS Mus musculus cDNA clone 621956 5′ TIGR cds;
NRNT(0.0): Mus musculus mRNA for IIGP protein;
Mouse DNA for Ig gamma-chain, secrete-type and membrane-bound, partial;
NRNT(2e-61): Mus musculus DNA for PSMB5, complete cds;
Homologous to sp P32507: poliovirus receptor homolog precursor;
Mouse Ig rearranged H-chain mRNA constant region;
M.musculus mRNA RHAMM;
R74638 MDB0793 Mouse brain, Stratagene Mus musculus cDNA 3′end;
Mus musculus pale ear (ep mutant allele) mRNA, partial cds;
mj35h09.r1 Soares mouse embryo NbME13.5 14.5 Mus musculus cDNA clone 4;
MUSGS00761 Mouse 3′-directed cDNA; MUSGS00761; clone mb1494. TIGR clus;
Homologous to sp P41725: brain enriched hyaluronan binding protein PRE;
M.musculus mRNA for D2A dopamine receptor;
mo54b05.r1 Life Tech mouse embryo 10 5 dpc 10665016 Mus musculus cDNA cds;
Mus musculus Bop1 mRNA, complete cds;
C75959 Mouse 3.5-dpc blastocyst cDNA Mus musculus cDNA clone J0001C05;
vm06f11.r1 Knowles Solter mouse blastocyst B1 Mus musculus cDNA clone;
Mus musculus Major Histocompatibility Locus class II region;
Mus musculus capping protein beta-subunit isoform 1 mRNA, complete cds;
Mus musculus mRNA for peroxisomal integral membrane protein PMP34;
Mus musculus mRNA for JAB, complete cds;
Mouse interferon regulatory factor 1 mRNA, complete cds;
Mus musculus GTPase IGTP mRNA, complete cds;
Mouse spi2 proteinase inhibitor (spi2/eb1) mRNA, 3 end;
Homologous to sp Q01514: Interferon-Induced Guanylate-Binding Protein;
Homologous to sp P13765: HLA CLASS II histocompatibility antigen, DO B;
NRNT(3e-39): Human phosphatidylinositol (4,5)bisphosphate 5-phosphatase;
Mus musculus (clone U2) T-cell specific protein mRNA, complete cds;
Mus musculus mRNA for peroxisomal integral membrane protein PMP34;
M. musculus mRNA for macrophage mannose receptor; or
the concentration of progenitor stem cells in blood.

46. The method of claim 45, wherein the expression level of the genetic marker in the bodily sample is less than the expression level of the genetic marker measured in the control bodily sample, which indicates that the compound or agent antagonizes the signaling activity of the VLA-4 receptor, wherein the genetic marker comprises:
vm06f11.r1 Knowles Solter mouse blastocyst B1 Mus musculus cDNA clone;
Mus musculus Major Histocompatibility Locus class II region;
Mus musculus capping protein beta-subunit isoform 1 mRNA, complete cds;
Mus musculus mRNA for peroxisomal integral membrane protein PMP34;
Mus musculus mRNA for JAB, complete cds;
Mouse interferon regulatory factor 1 mRNA, complete cds;
Mus musculus GTPase IGTP mRNA, complete cds;
Mouse spi2 proteinase inhibitor (spi2/eb1) mRNA, 3 end;
Homologous to sp Q01514: Interferon-Induced Guanylate-Binding Protein;
Homologous to sp P13765: HLA Class II histocompatibility antigen, DO B;
NRNT(3e-39): Human phosphatidylinositol (4,5)bisphosphate 5-phosphatase;
Mus musculus (clone U2) T-cell specific protein mRNA. complete cds; or
M. musculus mRNA for macrophage mannose receptor.

47. The method of claim 45, wherein the expression level of the genetic marker in the bodily sample is greater than the expression level of the genetic marker measured in the control bodily sample, which indicates that the compound or agent antagonizes the signaling activity of the VLA-4 receptor, wherein the genetic marker comprises:
Mus musculus anti-von Willebrand factor antibody NMC-4 kappa chain mRNA;
Mouse gene for immunoglobulin alpha heavy chain, switch region and con;
(H-2 CLASS I histocompatibility antigen, D-K alpha chain precursor;
Mus musculus MHC class I Qa-1a antigen mRNA, complete cds;
Mus musculus ribosomal protein L41 mRNA, complete cds;
Mouse MHC class I D-region cell surface antigen (D2d) gene, complete c;
Mus musculus mRNA for erythroid differentiation regulator, partial;
NRNT(1e-92): , complete sequence [Mus musculus];
vc50e11.r1 Knowles Solter mouse 2 cell Mus musculus cDNA clone 778028;
NRNT(0.0): Mus musculus mRNA for IIGP protein;
Mouse DNA for Ig gamma-chain, secrete-type and membrane-bound, partial;
NRNT(2e-61): Mus musculus DNA for PSMB5, complete cds;
Homologous to sp P32507: Poliovirus Receptor Homolog Precursor;
Mouse Ig rearranged H-chain mRNA constant region;
M.musculus mRNA RHAMM;
R74638 MDB0793 Mouse brain, Stratagene Mus musculus cDNA 3′end;
Mus musculus pale ear (ep mutant allele) mRNA, partial cds;
mj35h09.r1 Soares mouse embryo NbME13.5 14.5 Mus musculus cDNA clone 4;
MUSGS00761 Mouse 3′-directed cDNA; MUSGS00761; clone mb1494. TIGR clus;
Homologous to sp P41725: brain enriched hyaluronan binding protein PRE;
M.musculus mRNA for D2A dopamine receptor;
mo54b05.r1 Life Tech mouse embryo 10 5 dpc 10665016 Mus musculus cDNA cds;
mt23g11.r1 Soares mouse 3NbMS Mus musculus cDNA clone 621956 5′ TIGR c;
Mus musculus Bop1 mRNA, complete cds;
C75959 Mouse 3.5-dpc blastocyst cDNA Mus musculus cDNA clone J0001C05; or
the concentration of progenitor stem cells in blood.

48. The method of claim 44, wherein the method is performed in a high thruput fashion.

49. A method for determining the efficacy of a potential antagonist of the signaling of a VLA-4 receptor, wherein such a method comprises the steps of:
(a) removing a first bodily sample from a mouse;
(b) measuring the level of Mus musculus mRNA for JAB, complete cds genetic marker in the first bodily sample;
(c) administering the potential antagonist to the mouse;
(d) removing a second bodily sample from the mouse;
(e) measuring the level of the genetic marker Mus musculus mRNA for JAB, complete cds in the second bodily sample; and
(f) comparing the measured levels of step (b) and step (e),
wherein a decrease in the level of the genetic marker measured in step (e) as compared to the level of the genetic marker measured in step (b) indicates the potential antagonist has efficacy in antagonizing the signaling activity of VLA-4.

50. The method of claim 49, wherein the first and second bodily samples comprise a bodily fluid, a bodily tissue, or a combination thereof.

51. The method of claim 49, wherein the potential antagonist comprises a protein, a nucleotide sequence, a chemical compound, or a combination thereof.

52. A method for determining the efficacy of a potential antagonist of the signaling of a VLA-4 receptor, wherein such a method comprises the steps of:
(a) removing a first bodily sample from a mouse;
(b) measuring the level of EST AA571535 genetic marker in the first bodily sample;
(c) administering the potential antagonist to the mouse;
(d) removing a second bodily sample from the mouse;
(e) measuring the level of the genetic EST AA571535 in the second bodily sample; and
(f) comparing the measured levels of step (b) and step (e),
wherein a decrease in the level of the genetic marker measured in step (e) as compared to the level of the genetic marker measured in step (b) indicates the potential antagonist has efficacy in antagonizing the signaling activity of VLA-4.

53. The method of claim 52, wherein the first and second bodily samples comprise a bodily fluid, a bodily tissue, or a combination thereof.

54. The method of claim 52, wherein the potential antagonist comprises a protein, a nucleotide sequence, a chemical compound, or a combination thereof.
